SEQUENCE LISTING (10 sequences)

SEQ ID NO: 1 - protein, 5 aa, synthetic construct
DYNVN

SEQ ID NO: 2 - protein, 17 aa, synthetic construct
VINPKYGTTRYNQKFKG

SEQ ID NO: 3 - protein, 7 aa, synthetic construct
GLNAWDY

SEQ ID NO: 4 - protein, 11 aa, synthetic construct
GASENIYGALN

SEQ ID NO: 5 - protein, 7 aa, synthetic construct
GATNLED

SEQ ID NO: 6 - protein, 9 aa, synthetic construct
QNVLTTPYT

SEQ ID NO: 7 - protein, 116 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 8 - protein, 107 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 9 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0345808 A1
(43) Pub. Date: Nov. 5, 2026

(54) ANTI-AVB6 ANTIBODIES AND ANTIBODY-DRUG CONJUGATES AND THEIR USE IN THE TREATMENT OF CANCER

(71) Applicant: Seagen Inc., New York, NY (US)

(72) Inventors: Natalya Nazarenko, Bothell, WA (US); Gabriela Patilea-Vrana, Bothell, WA (US); Shyra Jane Gardai, Bothell, WA (US); Robert Lyon, Bothell, WA (US); Vivian Trang, Bothell, WA (US); Mary Campbell, Bothell, WA (US); Scott Knowles, Bothell, WA (US)

(73) Assignee: Seagen Inc., New York, NY (US)

(21) Appl. No.: 19/124,350

(22) PCT Filed: Nov. 2, 2023
(86) PCT No.: PCT/US2023/078437

§ 371 Date: Apr. 25, 2025

(51) Int. Cl. A61K 47/68; A61K 9/00; A61K 31/282; A61K 33/243; A61K 39/00; A61K 39/395; A61P 35/00; C07K 16/28

(52) U.S. Cl. CPC A61K 47/6849; A61K 9/0019; A61K 31/282; A61K 33/243; A61K 39/3955; A61K 47/68031; A61K 47/6889; A61P 35/00; C07K 16/2839; A61K 2039/505; C07K 2317/24; C07K 2317/565

(57) ABSTRACT

Provided are methods and compositions for treating solid tumors, such as non-small cell lung cancer, head and neck cancer, breast cancer, esophageal cancer, ovarian cancer, skin cancer, pancreatic cancer, bladder cancer, cervical cancer, and gastric cancer, in a subject, such as by the administration of antibodies and antibody-drug conjugates that bind to alpha-v beta-6 (avβ6). Also provided are articles of manufacture and compositions comprising said antibodies and antibody drug-conjugates that bind to avβ6 for use in treating solid tumors (e.g., non-small cell lung cancer, head and neck cancer, breast cancer, esophageal cancer, ovarian cancer, skin cancer, pancreatic cancer, bladder cancer, cervical cancer, and gastric cancer).

### CROSS REFERENCE TO RELATED APPLICATIONS

This application claims the benefit of U.S. Provisional App. No. 63/422,098 filed Nov. 3, 2022 and U.S. Provisional App. No. 63/457,205 filed Apr. 5, 2023, each of which is incorporated by reference in its entirety for all purposes.

### REFERENCE TO A SEQUENCE LISTING

This application includes an electronic sequence listing in a file named AVB6-00412PC_Sequence_Listing created on Oct. 6, 2023 and containing 11 Kb, which is hereby incorporated by reference.

### TECHNICAL FIELD

The present invention relates to methods and compositions for treating solid tumors, such as non-small cell lung cancer, head and neck cancer, breast cancer, esophageal cancer, ovarian cancer, skin cancer, pancreatic cancer, bladder cancer, cervical cancer, and gastric cancer, in a subject, such as by the administration of antibodies and antibody-drug conjugates that bind to alpha-v beta-6 (αvβ6).

### BACKGROUND

αvβ6, which is also known as alpha-v beta-6, is a cell adhesion receptor that binds extracellular matrix proteins such as fibronectin. αvβ6 is composed of an alpha v subunit and a beta 6 subunit, and is upregulated in multiple cancers, including non-small cell lung cancer (NSCLC).

Subjects with metastatic or unresectable solid tumor malignancies who have disease that has relapsed or is refractory to all standard-of-care therapies or have disease for which current therapy is not considered to be providing benefit, i.e., those subjects who have exhausted all standard-of-care treatment, have a need for new therapeutic options with the potential of delaying the development of new metastatic lesions and/or controlling or reducing disease burden.

The present invention meets the need for improved treatment of solid tumors, such as non-small cell lung cancer, head and neck cancer, breast cancer, esophageal cancer, ovarian cancer, skin cancer, pancreatic cancer, bladder cancer, cervical cancer, and gastric cancer, by providing highly specific and effective anti-αvβ6 antibody-drug conjugates.

All references cited herein, including patent applications, patent publications, and scientific literature, are herein incorporated by reference in their entirety, as if each individual reference were specifically and individually indicated to be incorporated by reference.

### SUMMARY

Provided herein is a method of treating a solid tumor in a subject, the method comprising administering to the subject an antibody-drug conjugate that binds to alpha-v beta-6 (αvβ6) or an antigen-binding fragment thereof conjugated to a monomethyl auristatin or a functional analog thereof or a functional derivative thereof, wherein the antibody-drug conjugate is administered at a dose ranging from about 0.7 mg/kg to about 2.5 mg/kg of the subject's body weight, and wherein the anti-αvβ6 antibody or antigen-binding fragment of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
   wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6. In some embodiments, the anti-αvβ6 antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO: 8. In some embodiments, the anti-αvβ6 antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8. In some embodiments, the anti-αvβ6 antibody or antigen-binding fragment thereof of the antibody-drug conjugate comprises a heavy chain comprising the amino acid sequence of SEQ ID NO:9 and a light chain comprising the amino acid sequence of SEQ ID NO: 10. In some embodiments, the monomethyl auristatin is monomethyl auristatin E (MMAE). In some embodiments, the anti-αvβ6 antibody or antigen-binding fragment thereof of the antibody-drug conjugate is a monoclonal antibody or a monoclonal antigen-binding fragment thereof. In some embodiments, the antibody-drug conjugate further comprises a linker between the anti-αvβ6 antibody or antigen-binding fragment thereof and the monomethyl auristatin. In some embodiments, the linker is a cleavable peptide linker. In some embodiments, the cleavable peptide linker has a formula: -MC-vc-PAB-, wherein:
a) MC is:

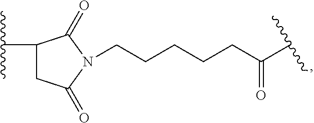 [SMILES: *C(=O)CCCCCN1C(=O)CC(*)C1=O]

b) vc is the dipeptide valine-citrulline, and
c) PAB is:

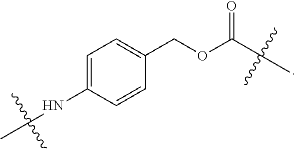 [SMILES: *Nc1ccc(COC(*)=O)cc1]

In some embodiments, the linker is attached to sulphydryl residues of the anti-αvβ6 antibody obtained by partial reduction or full reduction of the anti-αvβ6 antibody or antigen-binding fragment thereof. In some embodiments, the linker is attached to monomethyl auristatin E (MMAE), wherein the antibody-drug conjugate has the following structure:

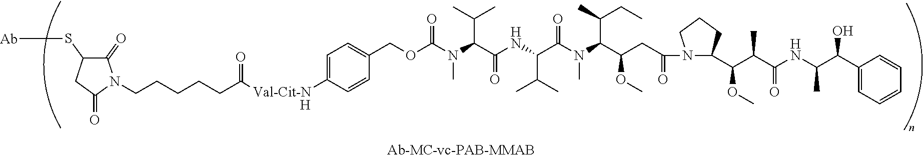

wherein p denotes a number from 1 to 8, S represents a sulphydryl residue of the anti-αvβ6 antibody, and Ab designates the anti-αvβ6 antibody or antigen-binding fragment thereof. In some embodiments, the average value of p in a population of the antibody-drug conjugates is about 4. In some embodiments, the dose is about 0.8 mg/kg, about 1.0 mg/kg, about 1.2 mg/kg, about 1.25 mg/kg, about 1.5 mg/kg, about 1.8 mg/kg, about 2.0 or about 2.4 mg/kg of the subject's body weight. In some embodiments, the dose is 0.8 mg/kg, 1.0 mg/kg, 1.2 mg/kg, 1.25 mg/kg, 1.5 mg/kg, 1.8 mg/kg, 2.0 or 2.4 mg/kg of the subject's body weight. In some embodiments, the dose is 1.8 mg/kg of the subject's body weight. In some embodiments, the dose is 1.25 mg/kg of the subject's body weight. In some embodiments, the dose is 1.5 mg/kg of the subject's body weight. In some embodiments, the subject's body weight is the subject's ideal body weight (IBW). In some embodiments, the subject's body weight is the subject's adjusted ideal body weight (AIBW). In some embodiments, the antibody-drug conjugate is administered once about every 1 week, once about every 2 weeks, once about every 3 weeks, or twice about every 3 weeks. In some embodiments, the antibody-drug conjugate is administered once about every 1 week. In some embodiments, the antibody-drug conjugate is administered once about every 2 weeks. In some embodiments, the antibody-drug conjugate is administered once about every 2 weeks at a dose of 1.8 mg/kg of the subject's body weight. In some embodiments, the antibody-drug conjugate is administered once about every 2 weeks at a dose of 1.5 mg/kg of the subject's body weight. In some embodiments, the antibody-drug conjugate is administered once about every 3 weeks. In some embodiments, the antibody-drug conjugate is administered is once about every 1 week for 2 consecutive weeks followed by about a 1 week resting period without any administration of the antibody-drug conjugate so that each cycle time is about 21 days including the resting period. In some embodiments, the antibody drug conjugate is administered on Days 1 and 8 of each 21-day cycle with no administration on Day 15. In some embodiments, the antibody-drug conjugate is administered once about every 1 week for 2 consecutive weeks followed by about a 1 week resting period without any administration of the antibody-drug conjugate so that each cycle time is about 21 days including the resting period and the antibody-drug conjugate is administered at a dose of 1.25 mg/kg of the subject's body weight. In some embodiments, the antibody-drug conjugate is administered once about every 1 week for 2 consecutive weeks followed by about a 1 week resting period without any administration of the antibody-drug conjugate so that each cycle time is about 21 days including the resting period and the antibody-drug conjugate is administered at a dose of 1.5 mg/kg of the subject's body weight. In some embodiments, the subject has been previously treated for the solid tumor with one or more therapeutic agents and did not respond to treatment, relapsed after the treatment, or experienced disease progression during the treatment, wherein the one or more therapeutic agents is not the antibody-drug conjugate. In some embodiments, the solid tumor is locally advanced or metastatic. In some embodiments, the solid tumor is selected from the group consisting of lung cancer, head and neck cancer, breast cancer, esophageal cancer, gastroesophageal junction cancer, ovarian cancer, peritoneal cancer, or fallopian tube cancer, skin cancer, pancreatic cancer, bladder cancer, cervical cancer, and gastric cancer. In some embodiments, the lung cancer is non-small cell lung cancer. In some embodiments the non-small cell lung cancer is a squamous cell carcinoma. In some embodiments, the non-small cell lung cancer is a non-squamous cell carcinoma. In some embodiments, the solid tumor is lung cancer and the lung cancer does not have a known epidermal growth factor receptor (EGFR) or anaplastic lymphoma kinase (ALK) mutation. In some embodiments, the solid tumor is lung cancer and the lung cancer has a known epidermal growth factor receptor (EGFR) or anaplastic lymphoma kinase (ALK) mutation. In some embodiments, the solid tumor is lung cancer and the subjected received prior platinum-based therapy and prior therapy with a PD-1/PD-L1 inhibitor. In some embodiments, the solid tumor is lung cancer and the subject has not received prior therapy with a taxane. In some embodiments, the head and neck cancer is head and neck squamous cell cancer. In some embodiments, the solid tumor is head and neck cancer and the subjected received prior platinum-based therapy and prior therapy with a PD-1/PD-L1 inhibitor. In some embodiments, the breast cancer is HER2-negative breast cancer. In some embodiments, the solid tumor is breast cancer and the subject received 1 or more prior lines of therapy for the breast cancer. In some embodiments, the 1 or more prior lines of therapy included a taxane administered as either a single agent or in combination with a different agent. In some embodiments, the esophageal cancer is esophageal squamous cell carcinoma. In some embodiments, the solid tumor is esophageal cancer and the subject received prior platinum-based chemotherapy. In some embodiments, the esophageal cancer is esophageal adenocarcinoma. In some embodiments, the gastroesophageal junction cancer is gastroesophageal junction adenocarcinoma. In some embodiments, the subject received prior platinum-based chemotherapy. In some embodiments, the ovarian cancer is high grade serous epithelial ovarian cancer. In some embodiments, the skin cancer is cutaneous squamous cell carcinoma. In some embodiments, the solid tumor is skin cancer and the subject received prior therapy with a PD-1/PD-L1 inhibitor. In some embodiments, the pancreatic cancer is exocrine pancreatic adenocarcinoma. In some embodiments, the solid tumor is an advanced stage cancer. In some embodiments, the advanced stage cancer is a stage 3 or stage 4 cancer. In some embodiments, the advanced stage cancer is metastatic cancer. In some embodiments, the route of administration for the antibody-drug conjugate is intravenous. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express αvβ6. In some embodiments, one or more therapeutic effects in the subject is improved after administration of the antibody-drug conjugate relative to a baseline. In some embodiments, the one or more therapeutic effects is selected from the group consisting of: size of a tumor derived from the cancer, objective response rate, duration of response, time to response, progression free survival, and overall survival. In some embodiments, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the subject has one or more adverse events and is further administered an additional therapeutic agent to eliminate or reduce the severity of the one or more adverse events. In some embodiments, the subject is at risk of developing one or more adverse events and is further administered an additional therapeutic agent to prevent or reduce the severity of the one or more adverse events. In some embodiments, the antibody-drug conjugate is administered as a monotherapy. In some embodiments, the method further comprises administering to the subject one or more additional therapeutic agents. In some embodiments, the one or more additional therapeutic agents is a checkpoint inhibitor. In some embodiments, the checkpoint inhibitor is a PD-1 inhibitor or a PD-L1 inhibitor. In some embodiments, the PD-1 inhibitor is an anti-PD-1 antibody. In some embodiments, the anti-PD-1 antibody is pembrolizumab, or a biosimilar thereof. In some embodiments, the pembrolizumab or biosimilar thereof is administered at a dose of about 200 mg. In some embodiments, the pembrolizumab or biosimilar thereof is administered once about every 3 weeks. In some embodiments, the pembrolizumab or biosimilar thereof is administered at a dose of about 400 mg. In some embodiments, the pembrolizumab or biosimilar thereof is administered once about every 6 weeks. In some embodiments, the route of administration of the pembrolizumab or biosimilar thereof is intravenous. In some embodiments, prior to the treatment the tumor comprises one or more cells that express PD-L1. In some embodiments, the subject has a tumor that expresses PD-L1 with TPS≥1%. In some embodiments, the subject has a tumor that expresses PD-L1 with TPS≥20%. In some embodiments, the subject has a tumor that expresses PD-L1 with TPS≥50%. In some embodiments, the subject has a tumor that expresses PD-L1 with CPS≥1. In some embodiments, the subject has a tumor that expresses PD-L1 with CPS≥20. In some embodiments, the one or more additional therapeutic agents is a platinum-based agent. In some embodiments, the platinum-based agent is carboplatin or cisplatin. In some embodiments, the first dose of the antibody-drug conjugate is administered prior to the administration of the first dose of the one or more additional therapeutic agents. In some embodiments, the first dose of the antibody drug conjugate is administered at least 1 day, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, or at least 6 months prior to the administration of the first dose of the one or more additional therapeutic agents. In some embodiments, the subject is a human. In some embodiments, the antibody-drug conjugate is in a pharmaceutical composition comprising the antibody-drug conjugate and a pharmaceutical acceptable carrier.

Also provided herein is a kit comprising:
(a) a dosage ranging from about 0.7 mg/kg to about 2.5 mg/kg of an antibody-drug conjugate that binds to αvβ6, wherein the antibody-drug conjugate comprises an anti-αvβ6 antibody or an antigen-binding fragment thereof conjugated to a monomethyl auristatin or a functional analog thereof or a functional derivative thereof; and
(b) instructions for using the antibody drug conjugate according to any of the embodiments provided herein.

Also provided herein is the use of an antibody-drug conjugate that binds to αvβ6 for the manufacture of a medicament for use any of the embodiments provided herein, wherein the antibody-drug conjugate comprises an anti-αvβ6 antibody or an antigen-binding fragment thereof conjugated to a monomethyl auristatin or a functional analog thereof or a functional derivative thereof.

Also provided herein is an antibody-drug conjugate that binds to αvβ6 for use in any of the embodiments provided herein, wherein the antibody-drug conjugate comprises an anti-αvβ6 antibody or an antigen-binding fragment thereof conjugated to a monomethyl auristatin or a functional analog thereof or a functional derivative thereof.

### BRIEF DESCRIPTION OF THE DRAWINGS

FIG. 1 is a graph showing the best percentage change in target lesion SoD (sum of diameters) from baseline per RECIST v1.1 in the NSCLC subset of the dose escalation study. Q3W=administration once every 3 weeks. 2Q3W=administration twice every 3 weeks. 2Q4W=administration twice every 4 weeks. Q1W=administration once every week.

FIG. 2 is a graph showing the best percentage change in target lesion SoD from baseline per RECIST v1.1 in the esophageal cancer (EC) subset of the dose escalation study. Q3W=administration once every 3 weeks. 2Q3W=administration twice every 3 weeks. 2Q4W=administration twice every 4 weeks. Q1W=administration once every week.

FIG. 3 is a graph showing the best percentage change in target lesion SoD from baseline per RECIST v1.1 in the HNSCC subset of the dose escalation study. 2Q3W=administration twice every 3 weeks. 2Q4W=administration twice every 4 weeks. Q1W=administration once every week.

FIG. 4 is a graph showing the best percentage change in target lesion SoD from baseline per RECIST v1.1 in the HNSCC 2Q3W subset of the dose expansion study. 2Q3W=administration twice every 3 weeks.

FIG. 5A is a Kaplan-Meier plot showing percent survival over time, where 4-fold tumor increase is the survival endpoint of mice subcutaneously implanted with syngeneic Renca cells engineered to express human integrin beta-6. FIG. 5B is a series of graphs showing tumor growth over time in individual mice treated with a mouse surrogate of SGN-B6A (mSGN-B6A), a mouse anti-PD-1 antibody (anti-mPD1), or both. All test articles were dosed weekly for 3 doses. Black triangles show the dosing schedule for mSGN-B6A. Blue/Grey triangles show the dosing schedule for anti-mPD1.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “αvβ6,” “avb6,” “alpha-v beta-6,” or “β6” are used interchangeably herein, and, unless specified otherwise, include any variants, isoforms and species homologs of human αvβ6 which are generally expressed by cells or expressed on cells transfected with the αvβ6 gene.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (CH or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, CL. The CL can be of κ (kappa) or λ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence, are produced from mouse B-cell fusions. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to αvβ6 is substantially free of antibodies that bind specifically to antigens other than αvβ6). An isolated antibody that binds specifically to αvβ6 can, however, have cross-reactivity to other antigens, such as αvβ6 molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-αvβ6 antibody includes a conjugate of an anti-αvβ6 antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-αvβ6 antibody is an antibody that binds to the antigen αvβ6.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, SnapGene Align, or ClustalW BioEdit software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y
where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate, which in the context of the present invention refers to an anti-αvβ6 antibody, which is coupled to a drug moiety (e.g., MMAE or MMAF) as described in the present application.

The abbreviations “vc” and “val-cit” refer to the dipeptide valine-citrulline.

The abbreviation VKG refers to the tripeptide linker valine-lysine-glycine.

The abbreviation “PAB” refers to the self-immolative spacer:

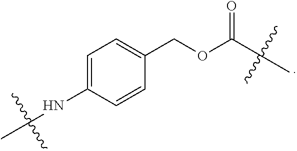 [SMILES: *Nc1ccc(COC(*)=O)cc1]

The abbreviation “MC” refers to the stretcher maleimidocaproyl:

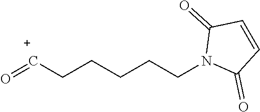 [SMILES: O=[C+]CCCCCN1C(=O)C=CC1=O]

The abbreviation “MP” refers to the stretcher maleimidopropionyl:

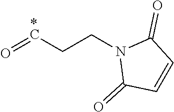

“PEG Unit” as used herein is an organic moiety comprised of repeating ethylene-oxy subunits (PEGs or PEG subunits) and may be polydisperse, monodisperse or discrete (i.e., having discrete number of ethylene-oxy subunits). Polydisperse PEGs are a heterogeneous mixture of sizes and molecular weights whereas monodisperse PEGs are typically purified from heterogeneous mixtures and are therefore provide a single chain length and molecular weight. Preferred PEG Units comprises discrete PEGs, compounds that are synthesized in step-wise fashion and not via a polymerization process. Discrete PEGs provide a single molecule with defined and specified chain length.

The PEG Unit provided herein comprises one or multiple polyethylene glycol chains, each comprised of one or more ethyleneoxy subunits, covalently attached to each other. The polyethylene glycol chains can be linked together, for example, in a linear, branched or star shaped configuration. Typically, at least one of the polyethylene glycol chains prior to incorporation into a camptothecin conjugate is derivatized at one end with an alkyl moiety substituted with an electrophilic group for covalent attachment to the carbamate nitrogen of a methylene carbamate unit (i.e., represents an instance of R). Typically, the terminal ethyleneoxy subunit in each polyethylene glycol chains not involved in covalent attachment to the remainder of the Linker Unit is modified with a PEG Capping Unit, typically an optionally substituted alkyl such as —CH₃, CH₂CH₃ or CH₂CH₂CO₂H. A preferred PEG Unit has a single polyethylene glycol chain with 2 to 24 —CH₂CH₂O— subunits covalently attached in series and terminated at one end with a PEG Capping Unit.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor.

The term “antibody-dependent cellular cytotoxicity”, or ADCC, is a mechanism for inducing cell death that depends upon the interaction of antibody-coated target cells with immune cells possessing lytic activity (also referred to as effector cells). Such effector cells include natural killer cells, monocytes/macrophages and neutrophils. The effector cells attach to an Fc effector domain(s) of Ig bound to target cells via their antigen-combining sites. Death of the antibody-coated target cell occurs as a result of effector cell activity.

The term “antibody-dependent cellular phagocytosis”, or ADCP, refers to the process by which antibody-coated cells are internalized, either in whole or in part, by phagocytic immune cells (e.g., macrophages, neutrophils and dendritic cells) that bind to an Fc effector domain(s) of Ig.

The term “complement-dependent cytotoxicity”, or CDC, refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

A “cytostatic effect” refers to the inhibition of cell proliferation. A “cytostatic agent” refers to an agent that has a cytostatic effect on a cell, thereby inhibiting the growth and/or expansion of a specific subset of cells. Cytostatic agents can be conjugated to an antibody or administered in combination with an antibody.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days.

A therapeutically effective amount of a drug (e.g., anti-αvβ6 antibody-drug conjugate) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an anti-αvβ6 antibody-drug conjugate) that is lower than the usual or typical dose of the therapeutic compound when administered alone for the treatment of a hyperproliferative disease (e.g., cancer).

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration for the anti-αvβ6 antibody-drug conjugate include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy (e.g., an anti-αvβ6 antibody-drug conjugate as described herein) or at the beginning of administration of the therapy. The baseline value can be compared to a reference value in order to determine the reduction or improvement of a symptom of a αvβ6-associated disease contemplated herein (e.g., cancer). The terms “reference” or “reference value” used interchangeably herein can refer to a measurement or characterization of a symptom after administration of the therapy (e.g., an anti-αvβ6 antibody-drug conjugate as described). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the anti-αvβ6 antibody-drug conjugate is the only anti-cancer agent administered to the subject during the treatment cycle.

Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

Reference to “about” a value or parameter herein includes (and describes) embodiments that are directed to that value or parameter per se. For example, description referring to “about X” encompasses and describes “X.”

The term “Combined Positive Score” or “CPS” as used herein, refers to an immunohistochemical method of measuring PD-L1 expression in a cancer, such as a tumor sample from a cancer. CPS is the number of PD-L1 staining cells (tumor cells, lymphocytes, macrophages) divided by the total number of viable tumor cells, multiplied by 100. For some therapeutic treatments, a tumor sample is considered to have PD-L1 expression if CPS ≥1. For example, a CPS ≥1 is required for a subject to be eligible for certain PD-1 or PD-L1 inhibitor therapies, such as subjects with gastric cancer, cervical cancer, and head and neck squamous cell cancer. In some instances, a CPS ≥10 is required for a subject to be eligible for certain PD-1 or PD-L1 inhibitor therapies, such as subjects with urothelial cancer (bladder cancer), esophageal squamous cell carcinoma (ESCC), or triple-negative breast cancer being treated with pembrolizumab.

The term “Tumor Proportion Score” or “TPS” as used herein, refers to an immunohistochemical method of measuring PD-L1 expression in a cancer, such as a tumor sample from a cancer. TPS is the percentage of viable tumor cells showing partial or complete membrane staining at any intensity. For some therapeutic treatments, a tumor sample is considered to have PD-L1 expression if TPS ≥1% and high PD-L1 expression if TPS ≥50%.

For example, a TPS ≥1% is the required for a subject to be eligible for certain PD-1 or PD-L1 inhibitor therapies (e.g., pembrolizumab), such as subjects with non-small cell lung cancer. In some instances, a TPS ≥50% is the required for a subject to be eligible for certain PD-1 or PD-L1 inhibitor therapies (e.g., cemiplimab).

The term “ideal body weight” or “IBW” as used herein, refers to a size descriptor that is unrelated to total body weight. IBW is an estimate of weight corrected for sex and height, and optionally frame size. IBW can be calculated, for example, using the formulas IBW=0.9H-88 (for males) and IBW=0.9H-92 (for females), wherein H=height in cm. Alternatively, IBW can also be calculated, for example using the following formulas: IBW (men)=50 kg+2.3 kg×(height, in −60); IBW (women)=45.5 kg+2.3 kg×(height, in −60).

The term “adjusted ideal body weight” or “AIBW” as used herein refers to a size descriptor that accounts for sex, total body weight, and height. AIBW can be calculated, for example, using the formula AIBW=IBW+0.4 (weight in kg−IBW).

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. General

The invention provides antibodies that specifically bind αvβ6. The present invention is based, in part, on the discovery that antibody-drug conjugates, including vcMMAE antibody-drug conjugates, targeted to αvβ6 are particularly effective at killing αvβ6+expressing cells. αvβ6 has been shown to be expressed in a variety of cancers, including non-small cell lung cancer (NSCLC) (squamous and adeno), head and neck cancer (including head and neck squamous carcinoma), esophageal cancer, breast cancer (including breast invasive carcinoma), ovarian cancer, bladder cancer (including urothelial carcinoma), skin cancer (squamous cell carcinoma, or SCC), renal cancer (including renal clear cell, renal papillary cell, and kidney chromophobe), cervical cancer, gastric cancer, prostate cancer (including prostate adenocarcinoma), endometrial cancer (including uterine carcinosarcoma and uterine corpus endometrial), rectum adenocarcinoma, thyroid carcinoma, colon adenocarcinoma, stomach adenocarcinoma, and pancreatic cancer (including pancreatic adenocarcinoma). The present invention provides an anti-αvβ6 antibody-drug conjugate that binds to αvβ6 for use in the treatment of non-small cell lung cancer, head and neck cancer (such as head and neck squamous carcinoma), breast cancer (such as advanced HER2-negative breast cancer), esophageal cancer (such as esophageal squamous cell carcinoma), ovarian cancer (such as high grade serous epithelial ovarian cancer), skin cancer (such as cutaneous squamous cell carcinoma), pancreatic cancer (such as exocrine pancreatic adenocarcinoma), bladder cancer, cervical cancer, and gastric cancer. In some embodiments, the cancer is non-small cell lung cancer. In some embodiments the non-small cell lung cancer is a squamous cell carcinoma. In some embodiments, the non-small cell lung cancer is a non-squamous cell carcinoma. In some embodiments, the cancer is head and neck cancer (such as head and neck squamous carcinoma). In some embodiments, the cancer is breast cancer (such as advanced HER2-negative breast cancer). In some embodiments, the cancer is esophageal cancer (such as esophageal squamous cell carcinoma). In some embodiments, the cancer is ovarian cancer (such as high grade serous epithelial ovarian cancer). In some embodiments, the cancer is skin cancer (such as cutaneous squamous cell carcinoma). In some embodiments, the cancer is pancreatic cancer (such as exocrine pancreatic adenocarcinoma). In some embodiments, the cancer is bladder cancer. In some embodiments, the cancer is cervical cancer. In some embodiments, the cancer is gastric cancer.

### III. Target Molecules

Unless otherwise indicated, αvβ6 refers to human αvβ6. An exemplary β6 human sequence is assigned GenBank accession number AAA36122. An exemplary αv human sequence is assigned NCBI NP_002201.1.

### IV. Antibodies of the Invention

Generally, anti-αvβ6 antibodies of the disclosure bind αvβ6, e.g., human αvβ6, and exert cytostatic and cytotoxic effects on malignant cells, such as non-small cell lung cancer, head and neck cancer (such as head and neck squamous carcinoma), breast cancer (such as advanced HER2-negative breast cancer), esophageal cancer (such as esophageal squamous cell carcinoma), ovarian cancer (such as high grade serous epithelial ovarian cancer), skin cancer (such as cutaneous squamous cell carcinoma), pancreatic cancer (such as exocrine pancreatic adenocarcinoma), bladder cancer, cervical cancer, and gastric cancer cells. Anti-αvβ6 antibodies of the disclosure are preferably monoclonal, and may be multispecific, human, humanized or chimeric antibodies, single chain antibodies, Fab fragments, F(ab′) fragments, fragments produced by a Fab expression library, and αvβ6 binding fragments of any of the above. In some embodiments, the anti-αvβ6 antibodies of the disclosure specifically bind αvβ6. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-αvβ6 antibodies are antigen-binding fragments as described herein and include, but are not limited to, Fab, Fab′ and F(ab′) 2, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-αvβ6 antibodies or antigen-binding fragments thereof are human, murine (e.g., mouse and rat), donkey, sheep, rabbit, goat, guinea pig, camelid, horse, or chicken.

The anti-αvβ6 antibodies of the present disclosure may be monospecific, bispecific, trispecific or of greater multi specificity. Multispecific antibodies may be specific for different epitopes of αvβ6 or may be specific for both αvβ6 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-αvβ6 antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Martin et al., “Modeling antibody hypervariable loops: a combined algorithm,” PNAS, 1989, 86 (23): 9268-9272, (“AbM” numbering scheme). The boundaries of a given CDR may vary depending on the scheme used for identification. In some embodiments, a “CDR” or “complementary determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

CDR sequences provided herein are according to the Kabat numbering scheme as described in Kabat et al. (1991), “Sequences of Proteins of Immunological Interest,” 5th Ed. Public Health Service, National Institutes of Health, Bethesda, MD (“Kabat” numbering scheme).

In certain embodiments anti-αvβ6 antibodies of the disclosure comprise one or more CDRs of an antibody described in WO 2021/113697. In certain embodiments antibodies of the disclosure comprise one or more CDRs of the humanized antibody anti-αvβ6 h2A2 HCLG. See WO 2021/113697. The disclosure encompasses an antibody or derivative thereof comprising a heavy or light chain variable domain, said variable domain comprising (a) a set of three CDRs, in which said set of CDRs are from monoclonal antibody h2A2 HCLG, and (b) a set of four framework regions, in which said set of framework regions differs from the set of framework regions in monoclonal antibody h2A2 HCLG, and in which said antibody or derivative thereof binds to αvβ6. In some embodiments, said antibody or derivative thereof specifically binds to αvβ6. In certain embodiments, the anti-αvβ6 antibody is h2A2 HCLG.

In one aspect, anti-αvβ6 antibodies that compete with h2A2 HCLG binding to αvβ6 are also provided herein. Anti-αvβ6 antibodies that bind to the same epitope as h2A2 HCLG are also provided herein.

In one aspect, provided herein is an anti-αvβ6 antibody comprising 1, 2, 3, 4, 5, or 6 of the CDR sequences of h2A2 HCLG.

In one aspect, provided herein is an anti-αvβ6 antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-αvβ6 antibody described herein may comprise any suitable framework variable domain sequence, provided that the antibody retains the ability to bind αvβ6 (e.g., human αvβ6). As used herein, heavy chain framework regions are designated “HC-FR1-FR4,” and light chain framework regions are designated “LC-FR1-FR4.”

In some embodiments of the anti-αvβ6 antibodies described herein, the heavy chain variable domain comprises the amino acid sequence of QFQLVQSGAEVKKPGASVKVSCKASGYSFTDYNVNWVRQAPGQGLEWIGVINPKYGT TRYNQKFKGRATLTVDKSTSTAYMELSSLRSEDTAVYYCTRGLNAWDYWGQGTLVTV SS (SEQ ID NO:7) and the light chain variable domain comprises the amino acid sequence of

(SEQ ID NO: 8)
DIQMTQSPSSLSASVGDRVTITCGASENIYGALNWYQQKPGKAPK
LLIYGATNLEDGVPSRFSGSGSGRDYTFTISSLQPEDIATYYCQN
VLTTPYTFGQGTKLEIK

In some embodiments of the anti-αvβ6 antibodies described herein, the heavy chain comprises the amino acid sequence of QFQLVQSGAEVKKPGASVKVSCKASGYSFTDYNVNWVRQAPGQGLEWIGVINPKYGT TRYNQKFKGRATLTVDKSTSTAYMELSSLRSEDTAVYYCTRGLNAWDYWGQGTLVTV SSASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKVEPKSCDKTHTCPPCPAPELL GGPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAKGQPREPQVYTLPP SRDELTKNQVSLTCLVKGFYPSDIAVEWESNGQPENNYKTTPPVLDSDGSFFLYSKLTV DKSRWQQGNVFSCSVMHEALHNHYTQKSLSLSPGK (SEQ ID NO:9) and the light chain comprises the amino acid sequence of

(SEQ ID NO: 10)
DIQMTQSPSSLSASVGDRVTITCGASENIYGALNWYQQKPGKAPK
LLIYGATNLEDGVPSRFSGSGSGRDYTFTISSLQPEDIATYYCQN
VLTTPYTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

In some embodiments of the anti-αvβ6 antibodies described herein, the heavy chain CDR sequences comprise the following:

a) CDR-H1:
(SEQ ID NO: 1)
DYNVN;
b) CDR-H2:
(SEQ ID NO: 2)
VINPKYGTTRYNQKFKG;
and
c) CDR-H3:
(SEQ ID NO: 3)
GLNAWDY.

In some embodiments of the anti-αvβ6 antibodies described herein, the light chain CDR sequences comprise the following:

a) CDR-L1:
(SEQ ID NO: 4)
GASENIYGALN;
b) CDR-L2:
(SEQ ID NO: 5)
GATNLED;
and
c) CDR-L3:
(SEQ ID NO: 6)
QNVLTTPYT

In one aspect, provided herein is an anti-αvβ6 antibody comprising a heavy chain variable domain comprising the amino acid sequence of SEQ ID NO:7 or comprising a light chain variable domain comprising the amino acid sequence of SEQ ID NO:8. In one aspect, provided herein is an anti-αvβ6 antibody comprising a heavy chain variable domain comprising the amino acid sequence of SEQ ID NO:7 and comprising a light chain variable domain comprising the amino acid sequence of SEQ ID NO:8.

In some embodiments, provided herein is an anti-αvβ6 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO: 7 contains substitutions (e.g., conservative substitutions), insertions, or deletions relative to the reference sequence and retains the ability to bind to a αvβ6 (e.g., human αvβ6). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDRs (i.e., in the FRs). In some embodiments, the anti-αvβ6 antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO: 3.

In some embodiments, provided herein is an anti-αvβ6 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO: 8 contains substitutions (e.g., conservative substitutions), insertions, or deletions relative to the reference sequence and retains the ability to bind to a αvβ6 (e.g., human αvβ6). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDRs (i.e., in the FRs). In some embodiments, the anti-αvβ6 antibody comprises a light chain variable domain sequence of SEQ ID NO:8 including post-translational modifications of that sequence. In a particular embodiment, the light chain variable domain comprises one, two or three CDRs selected from: (a) CDR-L1 comprising the amino acid sequence of SEQ ID NO:4, (b) CDR-L2 comprising the amino acid sequence of SEQ ID NO: 5, and (c) CDR-L3 comprising the amino acid sequence of SEQ ID NO:6.

In some embodiments, the anti-αvβ6 antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-αvβ6 antibody of the anti-αvβ6 antibody-drug conjugate comprises: i) a heavy chain CDR1 comprising the amino acid sequence of SEQ ID NO: 1, a heavy chain CDR2 comprising the amino acid sequence of SEQ ID NO: 2, a heavy chain CDR3 comprising the amino acid sequence of SEQ ID NO: 3; and ii) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 6.

In some embodiments, the anti-αvβ6 antibody of the anti-αvβ6 antibody-drug conjugate comprises: i) an amino acid sequence having at least 85% sequence identity to a heavy chain variable region comprising the amino acid sequence of SEQ ID NO: 7, and ii) an amino acid sequence having at least 85% sequence identity to a light chain variable region comprising the amino acid sequence of SEQ ID NO: 8.

In some embodiments, the anti-αvβ6 antibody of the anti-αvβ6 antibody-drug conjugate is a monoclonal antibody.

In some embodiments, the anti-αvβ6 antibody of the anti-αvβ6 antibody-drug conjugate is h2A2 HCLG as described in WO 2021/113697

Anti-αvβ6 antibodies of the present invention may also be described or specified in terms of their binding affinity to αvβ6 (e.g., human αvβ6). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The heavy and light chain variable regions of humanized antibodies can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 weak complement-dependent cytotoxicity and human. IgG4 lacks complement-dependent cytotoxicity. Human IgG1 and IgG3 also induce stronger cell mediated effector functions than human IgG2 and IgG4. Light chain constant regions can be lambda or kappa. Antibodies can be expressed as tetramers containing two light and two heavy chains, as separate heavy chains, light chains, as Fab, Fab′, F(ab′) 2, and Fv, or as single chain antibodies in which heavy and light chain variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

Exemplary substitution include the amino acid substitution of the native amino acid to a cysteine residue is introduced at amino acid position 234, 235, 237, 239, 267, 298, 299, 326, 330, or 332, preferably an S239C mutation in a human IgG1 isotype (US20100158909). The presence of an additional cysteine residue allows interchain disulfide bond formation. Such interchain disulfide bond formation can cause steric hindrance, thereby reducing the affinity of the Fc region-FcyR binding interaction. The cysteine residue(s) introduced in or in proximity to the Fc region of an IgG constant region can also serve as sites for conjugation to therapeutic agents (i.e., coupling cytotoxic drugs using thiol specific reagents such as maleimide derivatives of drugs. The presence of a therapeutic agent causes steric hindrance, thereby further reducing the affinity of the Fc region-FcγR binding interaction. Other substitutions at any of positions 234, 235, 236 and/or 237 reduce affinity for Fcγ receptors, particularly FcγRI receptor (see, e.g., U.S. Pat. Nos. 6,624,821, 5,624,821.)

The in vivo half-life of an antibody can also impact on its effector functions. The half-life of an antibody can be increased or decreased to modify its therapeutic activities. FcRn is a receptor that is structurally similar to MHC Class I antigen that non-covalently associates with β2-microglobulin. FcRn regulates the catabolism of IgGs and their transcytosis across tissues (Ghetie and Ward, 2000, Annu. Rev. Immunol. 18:739-766; Ghetie and Ward, 2002, Immunol. Res. 25:97-113). The IgG-FcRn interaction takes place at pH 6.0 (pH of intracellular vesicles) but not at pH 7.4 (pH of blood); this interaction enables IgGs to be recycled back to the circulation (Ghetie and Ward, 2000, Ann. Rev. Immunol. 18:739-766; Ghetie and Ward, 2002, Immunol. Res. 25:97-113). The region on human IgG1 involved in FcRn binding has been mapped (Shields et al, 2001, J. Biol. Chem. 276:6591-604). Alanine substitutions at positions Pro238, Thr256, Thr307, Gln311, Asp312, Glu380, Glu382, or Asn434 of human IgG1 enhance FcRn binding (Shields et al, 2001, J. Biol. Chem. 276:6591-604). IgG1 molecules harboring these substitutions have longer serum half-lives. Consequently, these modified IgG1 molecules may be able to carry out their effector functions, and hence exert their therapeutic efficacies, over a longer period of time compared to unmodified IgG1. Other exemplary substitutions for increasing binding to FcRn include a Gln at position 250 and/or a Leu at position 428. EU numbering is used for all position in the constant region.

Oligosaccharides covalently attached to the conserved Asn297 are involved in the ability of the Fc region of an IgG to bind FcyR (Lund et al, 1996, J. Immunol. 157:4963-69; Wright and Morrison, 199', Trends Biotechnol. 15:26-31). Engineering of this glycoform on IgG can significantly improve IgG-mediated ADCC. Addition of bisecting N-acetylglucosamine modifications (Umana et al, 1999, Nat. Biotechnol. 17:176-180; Davies et al, 2001, Biotech. Bioeng. 74:288-94) to this glycoform or removal of fucose (Shields et al, 2002, J. Biol. Chem. 277:26733-40; Shinkawa et al, 2003, J. Biol. Chem. 278:6591-604; Niwa et a/., 2004, Cancer Res. 64:2127-33) from this glycoform are two examples of IgG Fc engineering that improves the binding between IgG Fc and FcyR, thereby enhancing Ig-mediated ADCC activity.

A systemic substitution of solvent-exposed amino acids of human IgG1 Fc region has generated IgG variants with altered FcyR binding affinities (Shields et al, 2001, J. Biol. Chem. 276:6591-604). When compared to parental IgG1, a subset of these variants involving substitutions at Thr256/Ser298, Ser298/Glu333, Ser298/Lys334, or Ser298/Glu333 Lys334 to Ala demonstrate increased in both binding affinity toward FcγR and ADCC activity (Shields et al, 2001, J. Biol. Chem. 276:6591-604; Okazaki et al, 2004, J. Mol. Biol. 336:1239-49).

Complement fixation activity of antibodies (both C1q binding and CDC activity) can be improved by substitutions at Lys326 and Glu333 (Idusogie et al., 2001, J. Immunol. 166:2571-2575). The same substitutions on a human IgG2 backbone can convert an antibody isotype that binds poorly to C1q and is severely deficient in complement activation activity to one that can both bind C1q and mediate CDC (Idusogie et al, 2001, J. Immunol. 166:2571-75). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al, 1995, J. Immunol. 154:2226-36). Also, substituting Ser444 located close to the carboxy-terminal of IgG 1 heavy chain with Cys induced tail-to-tail dimerization of IgG 1 with a 200-fold increase of CDC activity over monomeric IgG1 (Shopes et al, 1992, J. Immunol. 148:2918-22). In addition, a bispecific diabody construct with specificity for C1q also confers CDC activity (Kontermann et al., 1997, Nat. Biotech. 15:629-31).

Complement activity can be reduced by mutating at least one of the amino acid residues 318, 320, and 322 of the heavy chain to a residue having a different side chain, such as Ala. Other alkyl-substituted non-ionic residues, such as Gly, He, Leu, or Val, or such aromatic non-polar residues as Phe, Tyr, Trp and Pro in place of any one of the three residues also reduce or abolish C1q binding. Ser, Thr, Cys, and Met can be used at residues 320 and 322, but not 318, to reduce or abolish C1q binding activity.

Replacement of the 318 (Glu) residue by a polar residue may modify but not abolish C1q binding activity. Replacing residue 297 (Asn) with Ala results in removal of lytic activity but only slightly reduces (about three-fold weaker) affinity for C1q. This alteration destroys the glycosylation site and the presence of carbohydrate that is required for complement activation. Any other substitution at this site also destroys the glycosylation site. The following mutations and any combination thereof also reduce C1q binding: D270A, K322A, P329A, and P31 IS (see WO 06/036291). The L234A/L235A mutation (or LALA mutation) also reduces C1q binding, as well as FcyR binding.

Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcgamma receptor binding or increase binding to FcRN.

The antibodies also include derivatives that are modified, i.e., by the covalent attachment of any type of molecule to the antibody such that covalent attachment does not prevent the antibody from binding to αvβ6 or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### V. Expression of Recombinant Antibodies

Humanized antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies.

Mammalian cells are a preferred host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines (e.g., DG44), various COS cell lines, HeLa cells, HEK293 cells, L cells, and non-antibody-producing myelomas including Sp2/0 and NS0. Preferably, the cells are nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., J. Immunol. 148:1149 (1992).

Once expressed, antibodies can be purified according to standard procedures of the art, including HPLC purification, column chromatography, gel electrophoresis and the like (see generally, Scopes, Protein Purification (Springer-Verlag, NY, 1982)).

### VI. Nucleic Acids

The invention further provides nucleic acids encoding any of the humanized heavy and light chains described above. Typically, the nucleic acids also encode a signal peptide fused to the mature heavy and light chains. Coding sequences on nucleic acids can be in operable linkage with regulatory sequences to ensure expression of the coding sequences, such as a promoter, enhancer, ribosome binding site, transcription termination signal and the like. The nucleic acids encoding heavy and light chains can occur in isolated form or can be cloned into one or more vectors. The nucleic acids can be synthesized by for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

In some aspects, also provided herein are nucleic acids encoding an anti-αvβ6 antibody or antigen-binding fragment thereof as described herein. Further provided herein are vectors comprising the nucleic acids encoding an anti-αvβ6 antibody or antigen-binding fragment thereof as described herein. Further provided herein are host cells expressing the nucleic acids encoding an anti-αvβ6 antibody or antigen-binding fragment thereof as described herein. Further provided herein are host cells comprising the vectors comprising the nucleic acids encoding an anti-αvβ6 antibody or antigen-binding fragment thereof as described herein.

The anti-αvβ6 antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

Monoclonal anti-αvβ6 antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., JMol, Biol., 222 (3): 581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

### VII. Antibody-Drug Conjugates

Anti-αvβ6 antibodies can be conjugated to cytotoxic or cytostatic moieties (including pharmaceutically compatible salts thereof) to form an antibody drug conjugate (ADC). Particularly suitable moieties for conjugation to antibodies are cytotoxic agents (e.g., chemotherapeutic agents), prodrug converting enzymes, radioactive isotopes or compounds, or toxins (these moieties being collectively referred to as a therapeutic agent). For example, an anti-αvβ6 antibody can be conjugated to a cytotoxic agent such as a chemotherapeutic agent, or a toxin (e.g., a cytostatic or cytocidal agent such as, e.g., abrin, ricin A, pseudomonas exotoxin, or diphtheria toxin).

An anti-αvβ6 antibody can be conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Arnon et al, “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al, “Antibodies For Drug Delivery,” in Controlled Drug Delivery (Robinson et al. eds., Marcel Dekker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al, 1982, Immunol. Rev. 62:119-58. See also, e.g., PCT publication WO 89/12624.)

The therapeutic agent can be conjugated in a manner that reduces its activity unless it is cleaved off the antibody (e.g., by hydrolysis, by antibody degradation or by a cleaving agent). Such therapeutic agent is attached to the antibody with a cleavable linker that is sensitive to cleavage in the intracellular environment of the αvβ6-expressing cancer cell but is not substantially sensitive to the extracellular environment, such that the conjugate is cleaved from the antibody when it is internalized by the αvβ6-expressing cancer cell (e.g., in the endosomal or, for example by virtue of pH sensitivity or protease sensitivity, in the lysosomal environment or in the caveolear environment).

Typically, the ADC comprises a linker region between the therapeutic agent and the anti-αvβ6 antibody. As noted supra, typically, the linker is cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Typically, the peptidyl linker is at least two amino acids long or at least three amino acids long. Cleaving agents can include cathepsins B and D and plasmin (see, e.g., Dubowchik and Walker, 1999, Pharm. Therapeutics 83:67-123). Most typical are peptidyl linkers that are cleavable by enzymes that are present in αvβ6-expressing cells. For example, a peptidyl linker that is cleavable by the thiol-dependent protease cathepsin-B, which is highly expressed in cancerous tissue, can be used (e.g., a linker comprising a Phe-Leu or a Gly-Phe-Leu-Gly peptide). Other such linkers are described, e.g., in U.S. Pat. No. 6,214,345. In specific embodiments, the peptidyl linker cleavable by an intracellular protease comprises a Val-Cit linker or a Phe-Lys dipeptide (see, e.g., U.S. Pat. No. 6,214,345, which describes the synthesis of doxorubicin with the Val-Cit linker). One advantage of using intracellular proteolytic release of the therapeutic agent is that the agent is typically attenuated when conjugated and the serum stabilities of the conjugates are typically high.

The cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al, 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929)).

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio) propionate), SPDB (N-succinimidyl-3-(2-pyridyldithio) butyrate) and SMPT (N-succinimidyl-oxycarbonyl-alpha-methyl-alpha-(2-pyridyl-dithio) toluene), SPDB and SMPT. (See, e.g., Thorpe et al, 1987, Cancer Res. 47:5924-5931; Wawrzynczak et al, In Immunoconjugates: Antibody Conjugates in Radioimagery and Therapy of Cancer (C. W. Vogel ed., Oxford U. Press, 1987. See also U.S. Pat. No. 4,880,935.)

The linker can also be a malonate linker (Johnson et al, 1995, Anticancer Res. 15:1387-93), a maleimidobenzoyl linker (Lau et al, 1995, Bioorg-Med-Chem. 3 (10): 1299-1304), or a 3′-N-amide analog (Lau et al, 1995, Bioorg-Med-Chem. 3 (10): 1305-12). The linker can also be a malonate linker (Johnson et al, 1995, Anticancer Res. 15:1387-93), a maleimidobenzoyl linker (Lau et al, 1995, Bioorg-Med-Chem. 3 (10): 1299-1304), or a 3′-N-amide analog (Lau et al, 1995, Bioorg-Med-Chem. 3 (10): 1305-12).

The linker also can be a non-cleavable linker, such as an maleimido-alkylene- or maleimide-aryl linker that is directly attached to the therapeutic agent (e.g., a drug). An active drug-linker is released by degradation of the antibody.

The linker can promote cellular internalization. The linker can promote cellular internalization when conjugated to the therapeutic agent (i.e., in the milieu of the linker-therapeutic agent moiety of the ADC or ADC derivative as described herein). Alternatively, the linker can promote cellular internalization when conjugated to both the therapeutic agent and the anti-αvβ6 antibody (i.e., in the milieu of the ADC as described herein).

The anti-αvβ6 antibody can be conjugated to the linker via a heteroatom of the antibody. These heteroatoms can be present on the antibody in its natural state or can be introduced into the antibody. In some aspects, the anti-αvβ6 antibody will be conjugated to the linker via a nitrogen atom of a lysine residue. In other aspects, the anti-αvβ6 antibody will be conjugated to the linker via a sulfur atom of a cysteine residue. The cysteine residue can be naturally-occurring or one that is engineered into the antibody. Methods of conjugating linkers and drug-linkers to antibodies via lysine and cysteine residues are known in the art.

Exemplary antibody-drug conjugates include auristatin based antibody-drug conjugates (i.e., the drug component is an auristatin drug). Auristatins bind tubulin, have been shown to interfere with microtubule dynamics and nuclear and cellular division, and have anticancer activity. Typically, the auristatin based antibody-drug conjugate comprises a linker between the auristatin drug and the anti-αvβ6 antibody. The linker can be, for example, a cleavable linker (e.g., a peptidyl linker, a carbohydrate linker) or a non-cleavable linker (e.g., linker released by degradation of the antibody). Auristatins include (but are not limited to) auristatin T, MMAF, and MMAE. The synthesis and structure of exemplary auristatins are described in U.S. Publication Nos. 7,659,241, 7,498,298, 2009-0111756, 2009-0018086, and 7,968, 687 each of which is incorporated herein by reference in its entirety and for all purposes.

Exemplary auristatin based antibody drug conjugates include vcMMAE (or 1006), vcMMAF and mcMMAF antibody drug conjugates as shown below wherein p represents the drug load, Ab is an anti-αvβ6 antibody as described herein and val-cit or “vc” represents the valine-citrulline dipeptide:

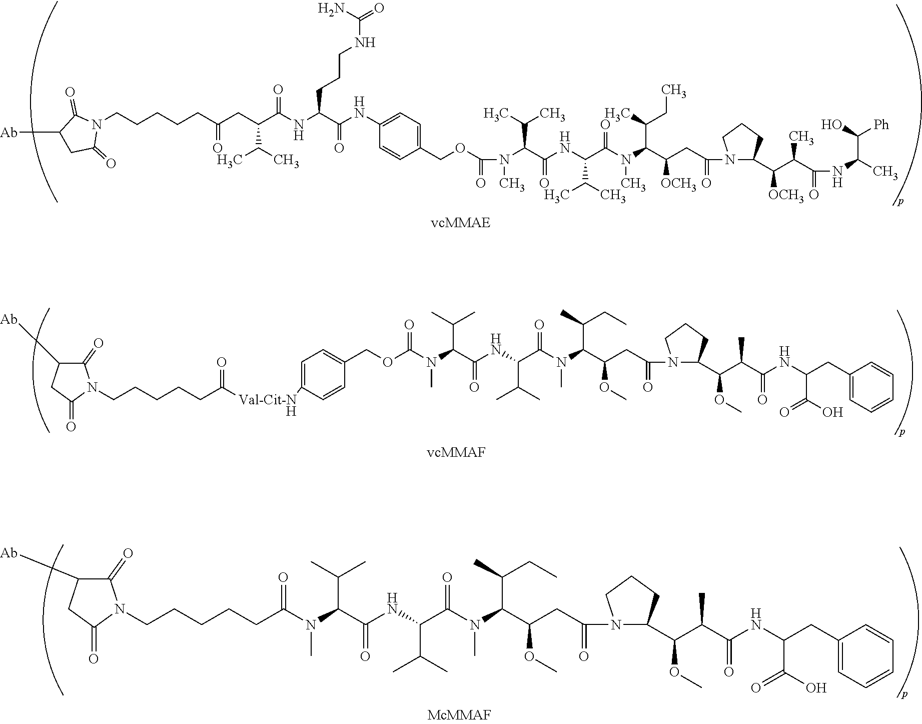

or a pharmaceutically acceptable salt thereof. The drug loading is represented by p, the number of drug-linker molecules per antibody. Referring to the αvβ6 targeted antibody-drug conjugates, the subscript p represents the drug load and, depending on the context, can represent the number of molecules of drug-linker molecules attached to an individual antibody molecule and as such, is an integer value, or can represent an average drug load and, as such, can be an integer or non-integer value but is typically a non-integer value. An average drug load represents the average number of drug-linker molecules per antibody in a population. Often, but not always, when we refer to an antibody, e.g., a monoclonal antibody, we are referring to a population of antibody molecules. In a composition comprising a population of antibody-drug conjugate molecules, the average drug load is an important quality attribute as it determines the amount of drug that can be delivered to a target cell. The percentage of unconjugated antibody molecules in the composition is included in the average drug load value.

In preferred aspects of the present invention, the average drug load (drug to antibody ratio (DAR)) when referring to a composition comprising a population of antibody-drug conjugate compounds is from about 1 to about 16, preferably about 2 to about 14, more preferably about 2 to about 10. In an embodiment, the DAR is from about 2 to about 5. In a further embodiment, the DAR is about 4. In a further embodiment, the DAR is 4. In another embodiment, the DAR is from about 6 to about 10. In a further embodiment, the DAR is about 8. In a further embodiment, the DAR is 8. The average number of drugs per antibody in a preparation may be characterized by conventional means such as mass spectroscopy, HIC, ELISA assay, and HPLC. In some aspects, the anti-αvβ6 antibody is attached to the drug-linker through a cysteine residue of the antibody. In some aspects, the cysteine residue is one that is engineered into the antibody. In other aspects, the cysteine residue is an interchain disulfide cysteine residue.

In some embodiments, incorporation of a polyethylene glycol polymer as a side chain into a cleavable β-glucuronide MMAE drug-linker provides antibody drug-conjugates with decreased plasma clearance and increased antitumor activity in xenograft models as compared to a non-PEGylated control. Accordingly, particularly advantageous drug-linkers for attachment to the antibodies of the present invention are as follows in formula V:

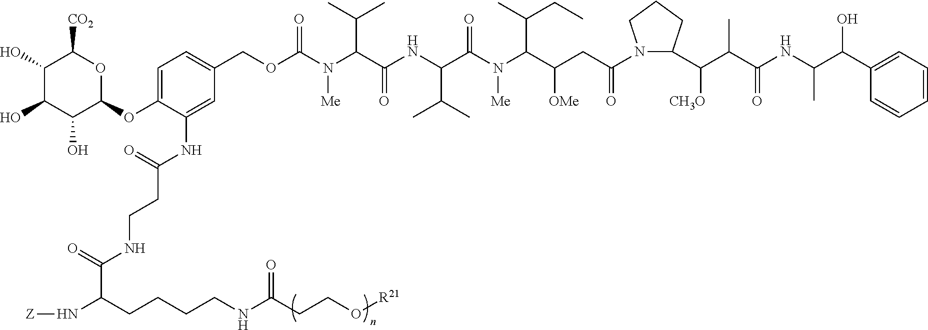

or a pharmaceutically acceptable salt thereof.

A preferred stereochemistry for such drug-linker is shown below in formula Va:

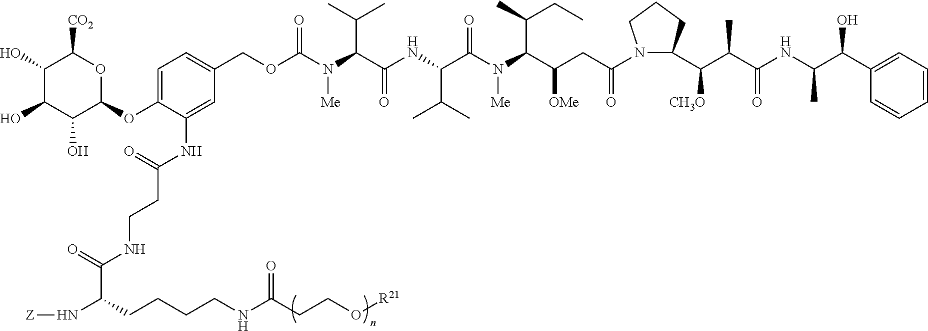

or a pharmaceutically acceptable salt thereof wherein for formulas V and Va, Z represents an organic moiety having a reactive site capable of reacting with a functional group on the antibody to form a covalent attachment thereto, n ranges from 8 to 36 and most preferably ranges from 8 to 14 (most preferably 12), R²¹ is a capping unit for the polyethylene glycol moiety, preferably —CH₃ or —CH₂CH₂CO₂H.

A preferred Z moiety is a maleimido-containing moiety. Particularly preferred Z moieties are shown in the drug-linkers below:

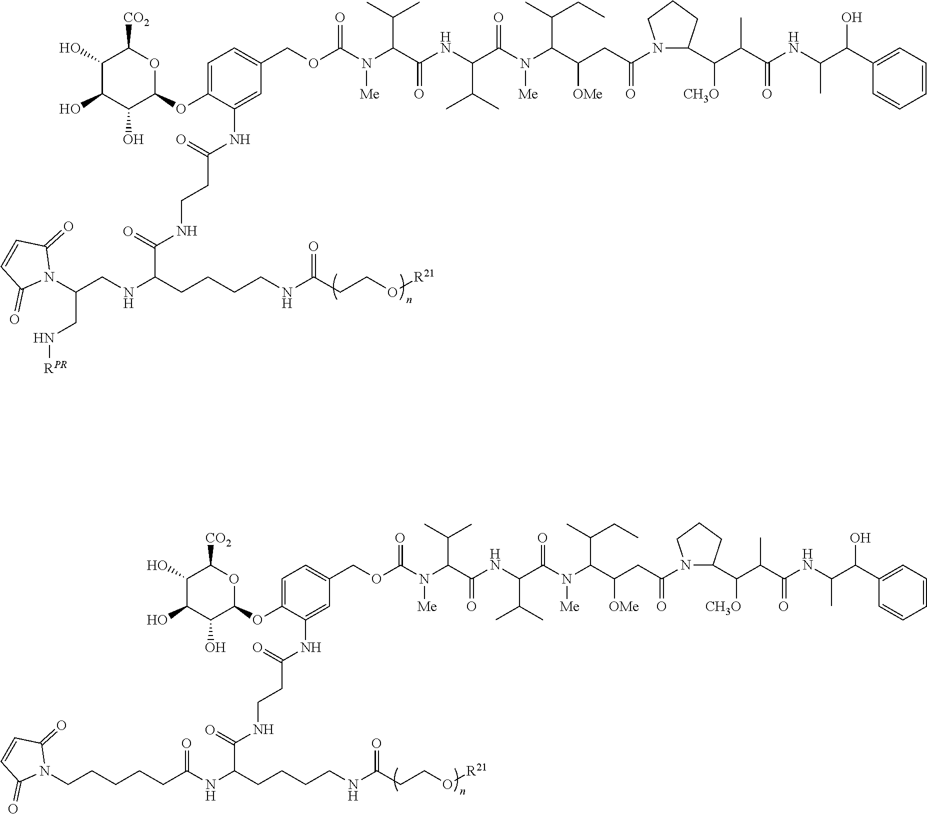

or a pharmaceutically acceptable salt thereof.

A preferred stereochemistry for such drug-linkers is shown below:

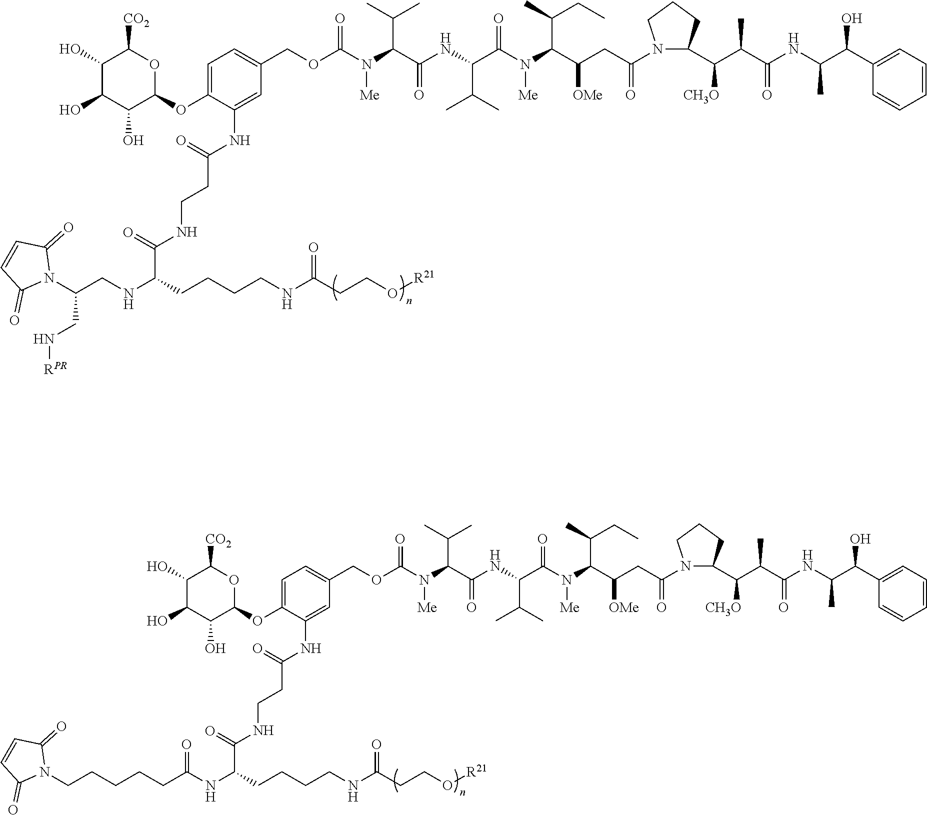

or a pharmaceutically acceptable salt thereof wherein for formulas VI, Via, VII and VIIa, n ranges from 8 to 36 and most preferably ranges from 8 to 14 (most preferably 12), R^{PR} is hydrogen or a protecting group, e.g., acid labile protecting group, e.g., BOC, R²¹ is a capping unit for the polyethylene glycol moiety, preferably —CH₃ or —CH₂CH₂CO₂H.

As noted above, R^{PR} can be hydrogen or a protecting group. Protective groups as used herein refer to groups which selectively block, either temporarily or permanently, a reactive site in a multifunctional compound. A protecting group is a suitable protecting group when it is capable of preventing or avoiding unwanted side-reactions or premature loss of the protecting group under reaction conditions required to effect desired chemical transformation elsewhere in the molecule and during purification of the newly formed molecule when desired, and can be removed under conditions that do not adversely affect the structure or stereochemical integrity of that newly formed molecule. Suitable amine protecting groups include acid-labile nitrogen protecting groups, including those provided by Isidro-Llobel et al. “Amino acid-protecting groups” Chem. Rev. (2009) 109:2455-2504. Typically, an acid-labile nitrogen-protecting group transforms a primary or secondary amino group to its corresponding carbamate and includes t-butyl, allyl, and benzyl carbamates.

As noted above, R²¹ is a capping unit for the polyethylene glycol moiety. As will be appreciated by the skilled artisan, polyethylene glycol units can be terminally capped with a wide diversity of organic moieties, typically those that are relatively non-reactive. Alkyl and substituted alkyl groups are preferred.

For the MMAE PEGylated ADCs, such as those exemplified herein, a particularly preferred average drug load is about 8. In exemplary embodiments, the drug-linkers are conjugated to the cysteine residues of the reduced inter-chain disulfides. In some aspects, the actual drug load for individual antibody molecules in the population of antibody-drug conjugate compounds is from 1 to 10 (or from 6 to 10 or from 6 to 8) with a predominant drug loading of 8. A higher drug load can be achieved, for example, if, in addition to the interchain disulfides, drug-linker is conjugated to introduced cysteine residues (such as a cysteine residue introduced at position 239, according to the EU index).

Exemplary ADCs include the following:

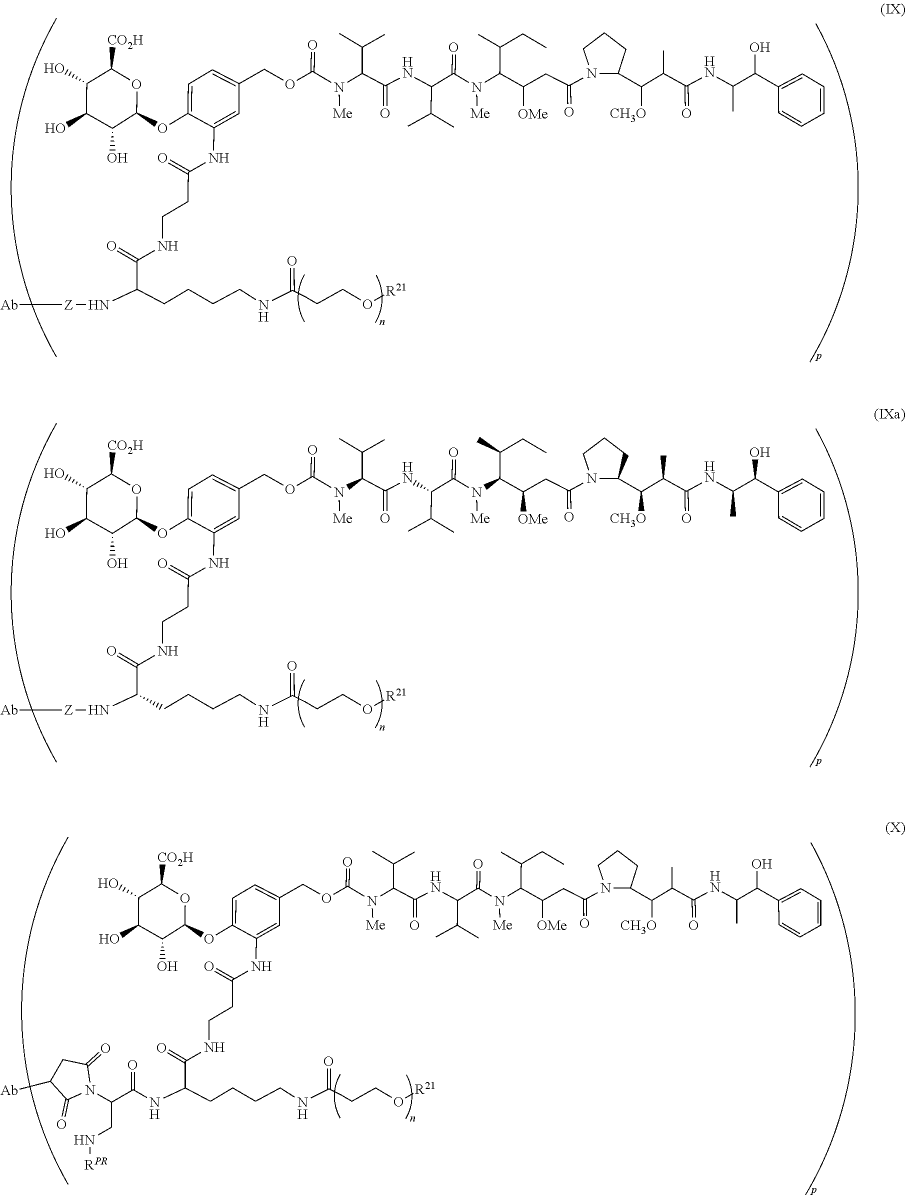

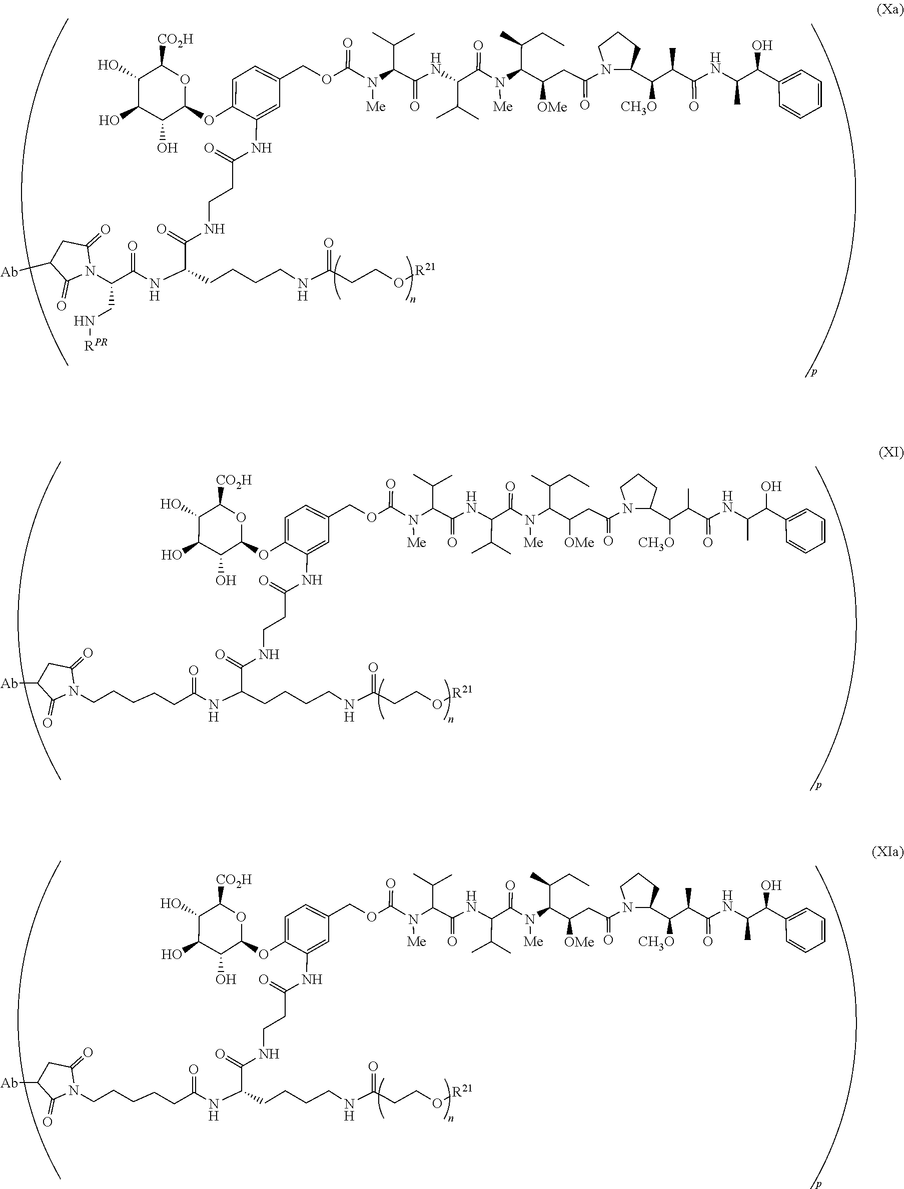

or a pharmaceutically acceptable salt thereof wherein n ranges from 8 to 36 and most preferably ranges from 8 to 14 (most preferably 12), R^{PR} is hydrogen or a protecting group, e.g., acid labile protecting group, e.g., BOC, R²¹ is a capping unit for the polyethylene glycol moiety, preferably —CH₃ or —CH₂CH₂CO₂H, Ab represents an anti-αVβ6 antibody and p represents an integer ranging from 1 to 16, preferably 1 to 14, 6 to 12, 6 to 10, or 8 to 10 when referring to individual antibody molecules or to an average drug load of from about 4 or about 6 to about 14, preferably about 8 when referring to a population of antibody molecules.

As noted above, the PEG (polyethylene glycol) portion of the drug linker can range from 8 to 36, however, it has been found that a PEG of 12 ethylene oxide units is particularly preferably. It has been found that longer PEG chains can result in slower clearance whereas shorter PEG chains can result in diminished activity. Accordingly, the subscript n in all of the embodiments above is preferably 8 to 14, 8 to 12, 10 to 12 or 10 to 14 and is most preferably 12.

Polydisperse PEGS, monodisperse PEGS and discrete PEGs can be used to make the PEGylated antibody drug conjugates of the present invention. Polydisperse PEGs are a heterogeneous mixture of sizes and molecular weights whereas monodisperse PEGs are typically purified from heterogenous mixtures and therefore provide a single chain length and molecular weight. Preferred PEG Units are discrete PEGs, compounds that are synthesized in step-wise fashion and not via a polymerization process. Discrete PEGs provide a single molecule with defined and specified chain length. As with the subscript “p”, when referring to populations of antibody-drug conjugates, the value for the subscript “n” can be an average number and can be an integer or non-integer number.

In preferred embodiments, covalent attachment of the antibody to the drug-linker is accomplished through a sulfhydryl functional group of the antibody interacting with a maleimide functional group of a drug linker to form a thio-substituted succinimide. The sulfhydryl functional group can be present on the Ligand Unit in the Ligand's natural state, for example, in a naturally-occurring residue (inter-chain disulfide resides), or can be introduced into the Ligand via chemical modification or by biological engineering, or a combination of the two. It will be understood that an antibody-substituted succinimide may exist in hydrolyzed form(s). For example, in preferred embodiments, an ADC is comprised of a succinimide moiety that when bonded to the antibody is represented by the structure of:

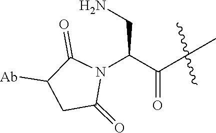

or is comprised of its corresponding acid-amide moiety that when bonded to the antibody is represented by the structure of:

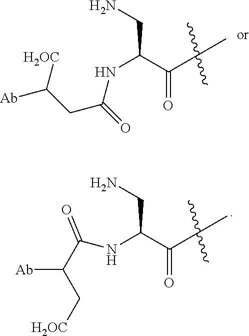

The wavy line indicates linkage to the remainder of the drug-linker.

In some embodiments, an anti-αvβ6 antibody of the invention is conjugated to monomethyl auristatin E via a MDpr-PEG(12)-gluc linker forming an antibody-drug conjugate having the structure:

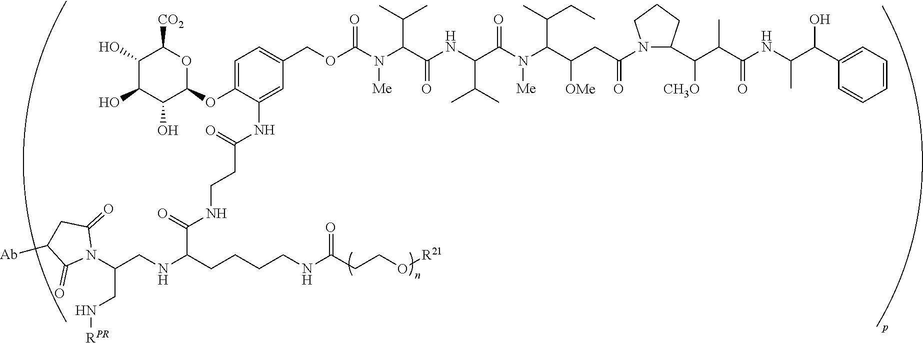

or a pharmaceutically acceptable salt thereof wherein n ranges from 8 to 36 and most preferably ranges from 8 to 14 (most preferably 12), R^{PR} is hydrogen or a protecting group, e.g., acid labile protecting group, e.g., BOC, R²¹ is a capping unit for the polyethylene glycol moiety, preferably —CH₃ or —CH₂CH₂CO₂H, Ab represents an anti-αVβ6 antibody and p represents an integer ranging from 1 to 16, preferably 1 to 14, 6 to 12, 6 to 10, or 8 to 10 when referring to individual antibody molecules or to an average drug load of from about 4 or about 6 to about 14, preferably about 8 when referring to a population of antibody molecules.

Exemplary antibody-drug conjugates also include camptothecin based antibody-drug conjugates (i.e., the drug component is a camptothecin drug). Camptothecins are topoisomerase inhibitors that have been shown to have anticancer activity. Typically the camptothecin based antibody-drug conjugate comprises a linker between the camptothecin drug and the anti-αvβ6 antibody. The linker can be, for example, a cleavable linker (e.g., a peptidyl linker, a carbohydrate linker) or a non-cleavable linker (e.g., linker released by degradation of the antibody). The synthesis and structure of exemplary camptothecin drug-linkers is described in PCT/US19/025968 (filed Apr. 5, 2019), which is incorporated herein by reference in its entirety and for all purposes.

Exemplary anti-αvβ6 antibody drug conjugates include camptothecin antibody drug conjugates as follows wherein p represents the drug load and Ab represents the anti-αvβ6 antibody:

In some embodiments, the camptothecin ADC has the formula (IC):

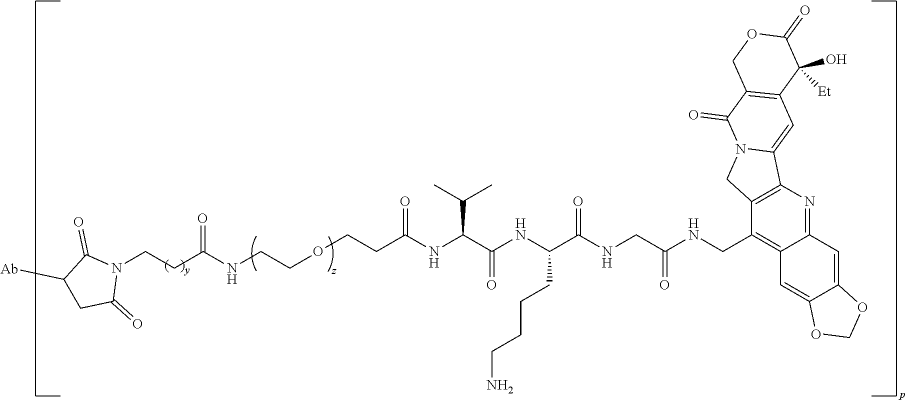

or a pharmaceutically acceptable salt thereof;
wherein
Ab is an anti-αvβ6 antibody;
y is 1, 2, 3, or 4, or is 1 or 4; and
z is an integer from 2 to 12, or is 2, 4, 8, or 12;
and p is 1-16.

In some aspect of these embodiments, p is 2, 3, 4, 5, 6, 7, 8, 9, or 10. In some aspect, p is 2, 4 or 8.

In some embodiments, the camptothecin ADC has the formula:

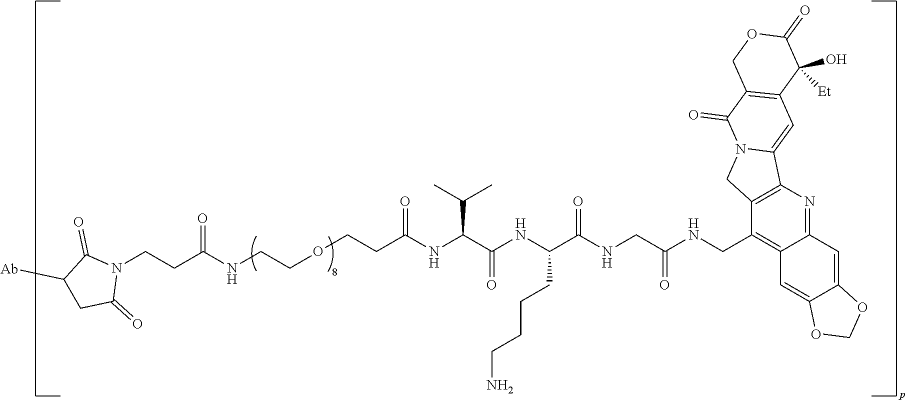

or a pharmaceutically acceptable salt thereof;
wherein p is 2, 4, or 8, preferably p is 8.

In some embodiments, the camptothecin ADC has the formula:

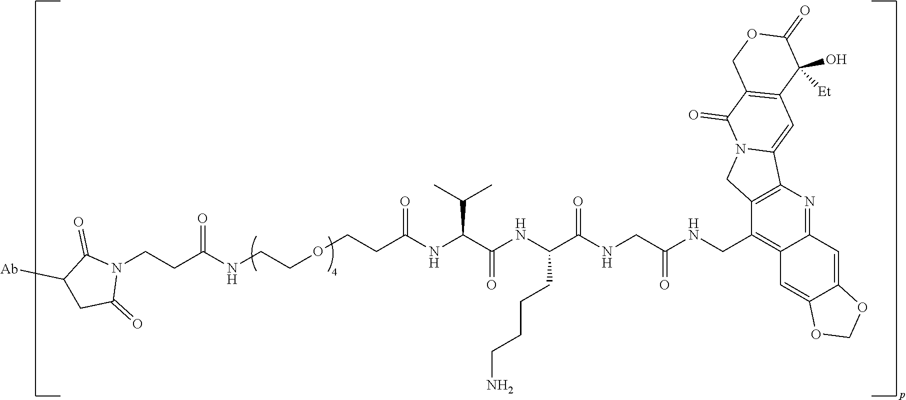

or a pharmaceutically acceptable salt thereof;
wherein p is 2, 4, or 8, preferably p is 8.

In some embodiments, the camptothecin drug-linker has the formula:

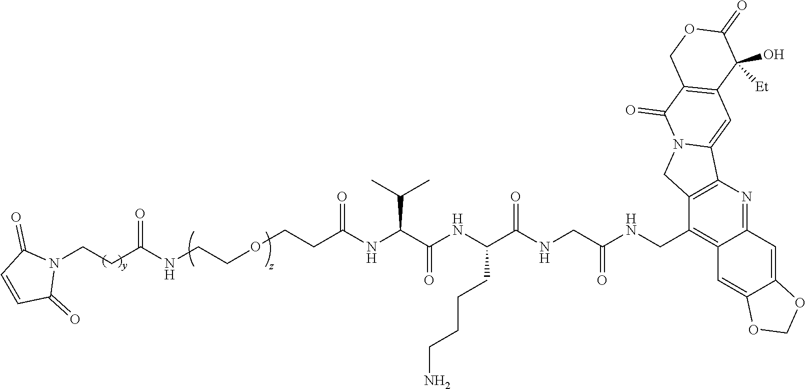

or a pharmaceutically acceptable salt thereof;
wherein
y is 1, 2, 3, or 4, or is 1 or 4; and
z is an integer from 2 to 12, or is 2, 4, 8, or 12.

In some embodiments, the camptothecin drug-linker has the formula:

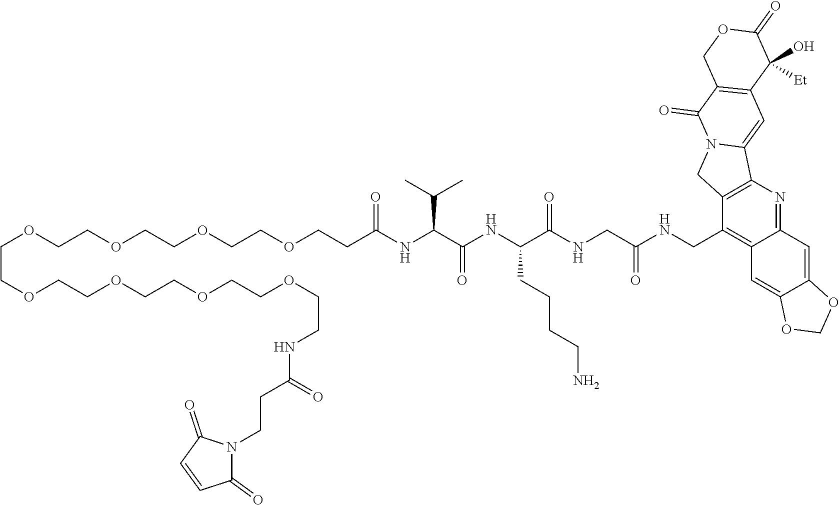 [SMILES: CC[C@@]1(O)C(=O)OCc2c1cc1n(c2=O)Cc2c-1nc1cc3c(cc1c2CNC(=O)CNC(=O)[C@H](CCCCN)NC(=O)[C@@H](NC(=O)CCOCCOCCOCCOCCOCCOCCOCCOCCNC(=O)CCN1C(=O)C=CC1=O)C(C)C)OCO3]

### MP-PEG8-VKG-Camptothecin

In some embodiments, the camptothecin drug-linker has the formula:

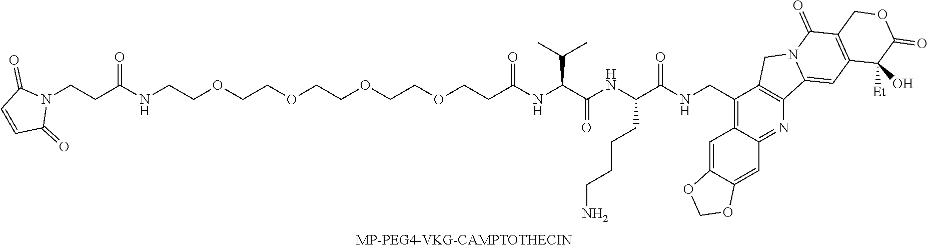 [SMILES: CC[C@@]1(O)C(=O)OCc2c1cc1n(c2=O)Cc2c-1nc1cc3c(cc1c2CNC(=O)[C@H](CCCCN)NC(=O)[C@@H](NC(=O)CCOCCOCCOCCOCCNC(=O)CCN1C(=O)C=CC1=O)C(C)C)OCO3]

In some embodiments, the camptothecin drug-linker has the formula:

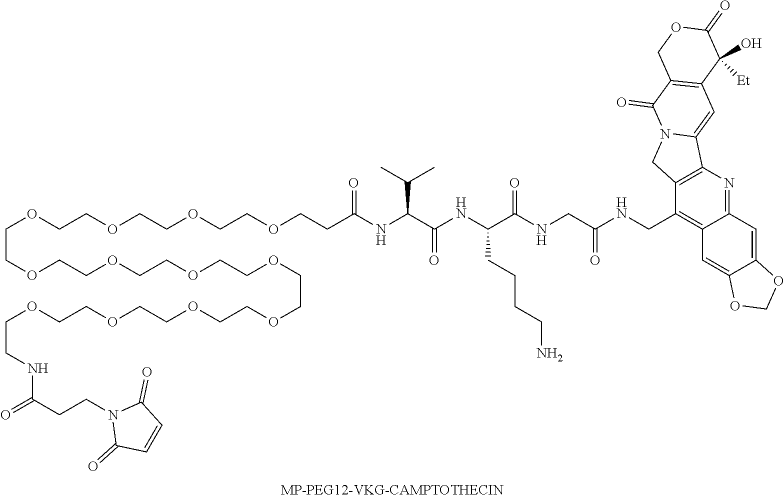 [SMILES: CC[C@@]1(O)C(=O)OCc2c1cc1n(c2=O)Cc2c-1nc1cc3c(cc1c2CNC(=O)CNC(=O)[C@H](CCCCN)NC(=O)[C@@H](NC(=O)CCOCCOCCOCCOCCOCCOCCOCCOCCOCCOCCOCCOCCNC(=O)CCN1C(=O)C=CC1=O)C(C)C)OCO3]

Other exemplary antibody-drug conjugates include maytansinoid antibody-drug conjugates (i.e., the drug component is a maytansinoid drug), and benzodiazepine antibody drug conjugates (i.e., the drug component is a benzodiazepine (e.g., pyrrolo[1,4]benzodiazepine dimers (PBD dimer), indolinobenzodiazepine dimers, and oxazolidinobenzodiazepine dimers)).

In some embodiments, a PBD dimer for use in the present invention is represented by formula I. The preferred stereochemistry of the PBD dimer is as shown in formula Ia:

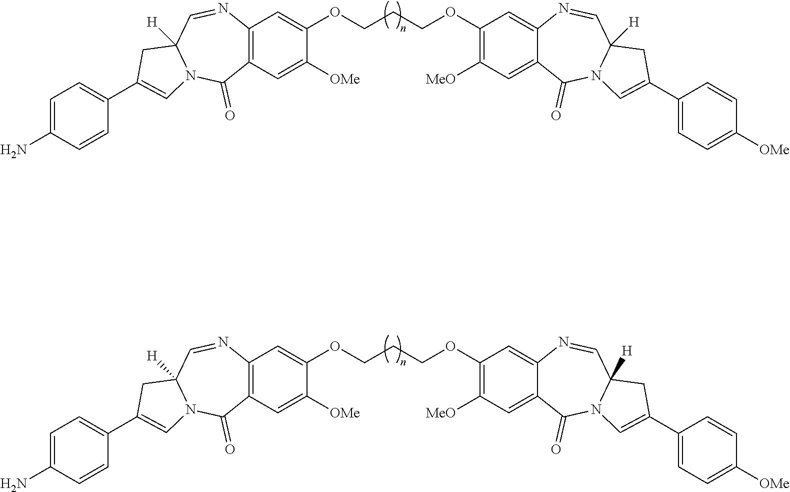

or a pharmaceutically salt, solvate, or solvate of the salt; wherein the subscript n is 1 or 3.

Solvates of formula (I) and (Ia) are typically formed from addition of water or alcoholic solvent across the imine functional group of one or both PBD monomers to form carbinolamine(s) and/or carbinolamine ethers. For example, at the N10-C11 position, there can be an imine (N═C), a carbinolamine (NH—CH(OH)), or a carbinolamine ether (NH—CH(Ome)) as represented by formulas I′ and Ia′ below:

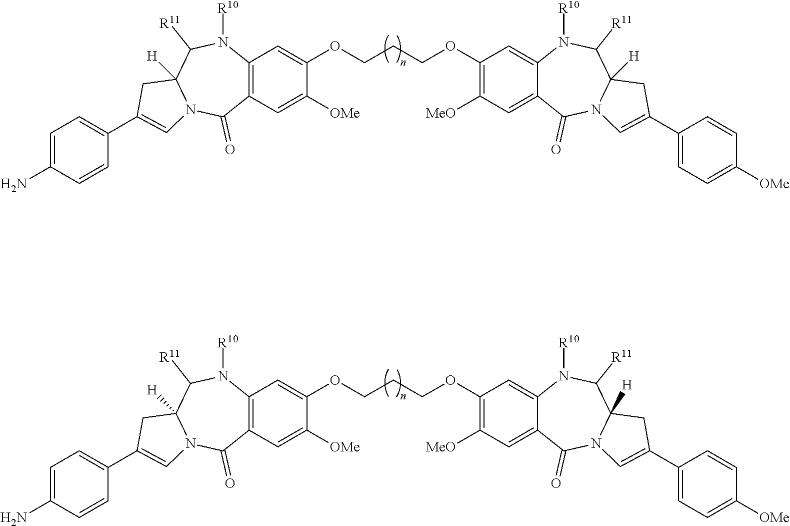

wherein either:
(a) R¹⁰ is H, and R¹¹ is OH or OR^{A}, where R^{A} is saturated C₁₋₄ alkyl (preferably methyl); or
(b) R¹⁰ and R¹¹ form a nitrogen-carbon double bond between the nitrogen and carbon atoms to which they are bound; or
   | one of R¹⁰ is H, and R¹¹ is OH or OR^{A}, where R^{A} is saturated C₁₋₄ alkyl (preferably methyl); and the other of R¹⁰ and R¹¹ form a nitrogen-carbon double bond between the nitrogen and carbon atoms to which they are bound.

The PBD dimer of formula I or Ia (or a pharmaceutically salt, solvate, or solvate of the salt thereof) is typically linked to the antibody via a Linker Unit, LU. The Linker Unit acts to release the PBD dimer of formula I or Ia (or a pharmaceutically salt, solvate, or solvate of the salt thereof) at the target site (e.g., inside the cancer cell). A PBD drug-linker compound for use in the present invention is represented below by formula II (preferred stereochemistry as shown in Ila) wherein LU is a Linker Unit. The Linker Unit can be, for example, a cleavable peptide Linker Unit (e.g., a linker comprising the valine-alanine peptide) or a cleavable disulfide Linker Unit:

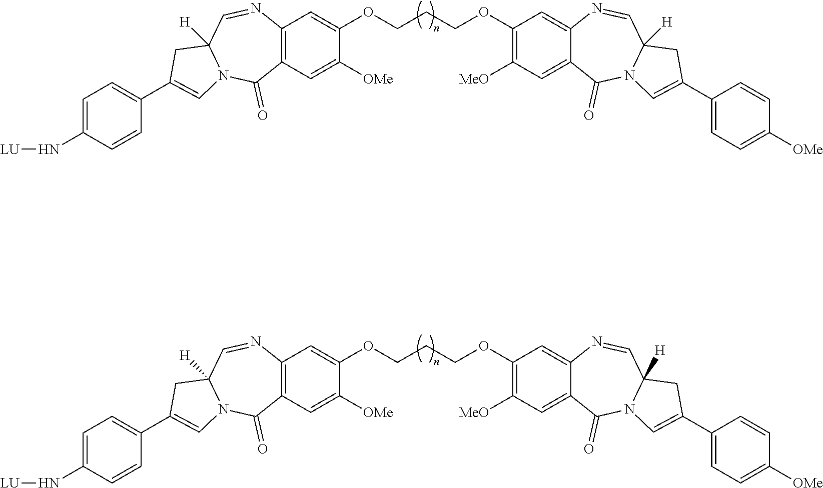

or a pharmaceutically salt, solvate, or solvate of the salt; wherein the subscript n is 1 or 3.

A preferred PBD drug-linker compound for use in the present invention is represented by Formula III below:

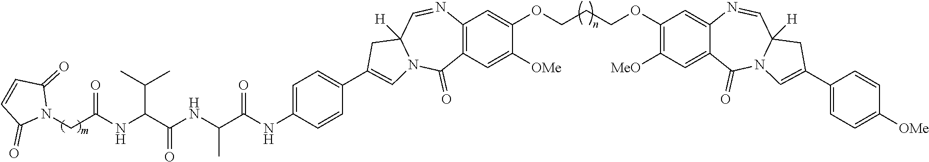

or a pharmaceutically salt, solvate, or solvate of the salt; wherein the subscript n is 1 or 3 and the subscript m is an integer from 2 to 5.

The PBD drug-linker is conjugated to an anti-αvβ6 antibody to produce a αvβ6 targeted antibody-drug conjugate. For example, the antibody can be conjugated to a drug-linker of formula II or formula III. An exemplary αvβ6 targeted antibody-drug conjugate is shown below in formulas IV, IVa, and IVb:

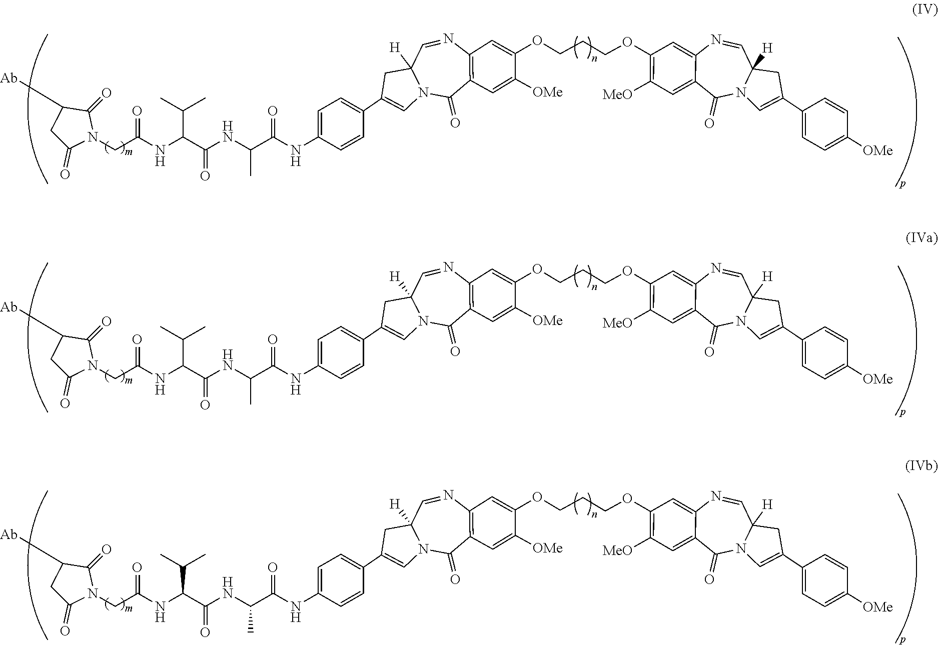

or a pharmaceutically salt, solvate, or solvate of the salt; wherein the subscript n is 1 or 3; the subscript m is an integer from 2 to 5; and the subscript p is from 1 to 4.

Useful classes of cytotoxic agents to conjugate to anti-αvβ6 antibodies include, for example, antitubulin agents, DNA minor groove binding agents, DNA replication inhibitors, chemotherapy sensitizers, or the like. Other exemplary classes of cytotoxic agents include anthracyclines, auristatins, camptothecins, duocarmycins, etoposides, maytansinoids and vinca alkaloids. Some exemplary cytotoxic agents include auristatins (e.g., auristatin T, auristatin E, AFP, monomethyl auristatin F (MMAF), lipophilic monomethyl aurstatin F, monomethyl auristatin E (MMAE)), DNA minor groove binders (e.g., enediynes and lexitropsins), duocarmycins, taxanes (e.g., paclitaxel and docetaxel), vinca alkaloids, nicotinamide phosphoribosyltranferase inhibitor (NAMPTi), tubulysin M, doxorubicin, morpholino-doxorubicin, and cyanomorpholino-doxorubicin.

The cytotoxic agent can be a chemotherapeutic such as, for example, doxorubicin, paclitaxel, melphalan, vinca alkaloids, methotrexate, mitomycin C or etoposide. The agent can also be a CC-1065 analogue, calicheamicin, maytansine, an analog of dolastatin 10, rhizoxin, or palytoxin.

The cytotoxic agent can also be an auristatin. The auristatin can be an auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatins include auristatin T, AFP, MMAF, and MMAE. The synthesis and structure of various auristatins are described in, for example, US 2005-0238649 and US2006-0074008.

The cytotoxic agent can be a DNA minor groove binding agent. (See, e.g., U.S. Pat. No. 6,130,237.) For example, the minor groove binding agent can be a CBI compound or an enediyne (e.g., calicheamicin).

The cytotoxic or cytostatic agent can be an anti-tubulin agent. Examples of anti-tubulin agents include taxanes (e.g., Taxol® (paclitaxel), Taxotere® (docetaxel)), T67 (Tularik), vinca alkyloids (e.g., vincristine, vinblastine, vindesine, and vinorelbine), and auristatins (e.g., auristatin E, AFP, MMAF, MMAE, AEB, AEVB). Exemplary auristatins are shown below in formulae III-XIII. Other suitable antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermoide and eleuthrobin.

The cytotoxic agent can be a maytansinoid, another group of anti-tubulin agents (e.g., DM1, DM2, DM3, DM4). For example, the maytansinoid can be maytansine or a maytansine containing drug linker such as DM-1 or DM-4 (ImmunoGen, Inc.; see also Chari et al., 1992, Cancer Res.)

### VIII. Therapeutic Applications

The anti-αvβ6 antibodies of the invention, alone or as anti-αvβ6 antibody-drug conjugates thereof, can be used to treat cancer. Some such cancers show detectable levels of αvβ6 measured at either the protein (e.g., by immunoassay, such as by using one of the exemplified antibodies) or mRNA level. Some such cancers show elevated levels of αvβ6 relative to noncancerous tissue of the same type, preferably from the same patient. An exemplary level of αvβ6 on cancer cells amenable to treatment is 5000-500,000 αvβ6 molecules per cell, although higher or lower levels can be treated. Optionally, a level of αvβ6 in a cancer is measured before performing treatment.

Examples of cancers associated with αvβ6 expression and amenable to treatment include non-small cell lung cancer (NSCLC) (squamous and adeno), head and neck cancer (including head and neck squamous carcinoma), esophageal cancer, breast cancer (including breast invasive carcinoma), ovarian cancer, bladder cancer (including urothelial carcinoma), skin cancer (squamous cell carcinoma, or SCC), renal cancer (including renal clear cell, renal papillary cell, and kidney chromophobe), cervical cancer, gastric cancer, prostate cancer (including prostate adenocarcinoma), endometrial cancer (including uterine carcinosarcoma and uterine corpus endometrial), rectum adenocarcinoma, thyroid carcinoma, colon adenocarcinoma, stomach adenocarcinoma, and pancreatic cancer (including pancreatic adenocarcinoma). In some embodiments, the antibodies or antibody-drug conjugates of the invention are used in methods of treating NSCLC. In some embodiments, the NSCLC is a squamous cell carcinoma. In some embodiments, the NSCLC is a non-squamous cell carcinoma. In some embodiments, the NSCLC is an adenocarcinoma. In some embodiments, the NSCLC does not harbor known mutations/alterations that confer eligibility for approved targeted therapies (e.g., epidermal growth factor receptor (EGFR), anaplastic lymphoma kinase (ALK), etc.). In some embodiments, the NSCLC has one or more known mutations/alterations that confer eligibility for approved targeted therapies (e.g., epidermal growth factor receptor (EGFR), anaplastic lymphoma kinase (ALK), etc.). In some embodiments, the NSCLC has a known EGFR mutation. In some embodiments, the NSCLC has a known ALK mutation. In some embodiments, the antibodies or antibody-drug conjugates of the invention are used in methods of treating head and neck cancer. In some embodiments, the head and neck cancer is a squamous cell carcinoma. In some embodiments, the antibodies or antibody-drug conjugates of the invention are used in methods of treating skin cancer. In some embodiments, the skin cancer is cutaneous squamous cell carcinoma. In some embodiments, the antibodies or antibody-drug conjugates of the invention are used in methods of treating esophageal cancer. In some embodiments, the esophageal cancer is esophageal squamous cell carcinoma. In some embodiments, the antibodies or antibody-drug conjugates of the invention are used in methods of treating breast cancer. In some embodiments, the breast cancer is a HER2-negative breast cancer. In some embodiments, the breast cancer is breast invasive carcinoma. In some embodiments, the antibodies or antibody-drug conjugates of the invention are used in methods of treating ovarian cancer. In some embodiments, the ovarian cancer is high grade serous epithelial ovarian cancer. In some embodiments, the antibodies or antibody-drug conjugates of the invention are used in methods of treating primary peritoneal cancer. In some embodiments, the antibodies or antibody-drug conjugates of the invention are used in methods of treating fallopian tube cancer. In some embodiments, the antibodies or antibody-drug conjugates of the invention are used in methods of treating bladder cancer. In some embodiments, the bladder cancer is urothelial carcinoma. In some embodiments, the antibodies or antibody-drug conjugates of the invention are used in methods of treating cervical cancer. In some embodiments, the antibodies or antibody-drug conjugates of the invention are used in methods of treating gastric cancer. In some embodiments, the antibodies or antibody-drug conjugates of the invention are used in methods of treating prostate cancer. In some embodiments, the prostate cancer is prostate adenocarcinoma. In some embodiments, the antibodies or antibody-drug conjugates of the invention are used in methods of treating renal cancer. In some embodiments, the renal cancer is renal clear cell carcinoma. In some embodiments, the renal cancer is renal papillary cell carcinoma. In some embodiments, the renal cancer is kidney chromophobe carcinoma. In some embodiments, the antibodies or antibody-drug conjugates of the invention are used in methods of treating endometrial cancer. In some embodiments, the endometrial cancer is uterine carcinosarcoma. In some embodiments, the endometrial cancer is uterine corpus endometrial carcinoma. In some embodiments, the antibodies or antibody-drug conjugates of the invention are used in methods of treating rectum adenocarcinoma. In some embodiments, the antibodies or antibody-drug conjugates of the invention are used in methods of treating thyroid carcinoma. In some embodiments, the antibodies or antibody-drug conjugates of the invention are used in methods of treating colon adenocarcinoma. In some embodiments, the antibodies or antibody-drug conjugates of the invention are used in methods of treating stomach cancer. In some embodiments, the stomach cancer is stomach adenocarcinoma. In some embodiments, the antibodies or antibody-drug conjugates of the invention are used in methods of treating pancreatic cancer. In some embodiments, the pancreatic cancer is exocrine pancreatic adenocarcinoma. The treatment can be applied to patients having locally advanced tumors of any of these kinds. The treatment can be applied to patients having primary or metastatic tumors of any of these kinds. The treatment can also be applied to patients having tumors of any of these kinds who are refractory to conventional treatments, or who have relapsed following a response to such treatments. In some embodiments, the subjects have received 1 or more prior lines of therapy to treat the tumor of any of these kinds. In some embodiments, the subjects have been previously treated for the tumor of any of these kinds with a platinum-based therapy. In some embodiments, the subjects have been previously treated for the tumor of any of these kinds with a PD-1/PD-L1 inhibitor. In some embodiments, the subjects have been previously treated for the tumor of any of these kinds with a taxane. In some embodiments, the subjects have not been previously treated for the tumor of any of these kinds with a taxane. In some embodiments, the subjects have been previously treated for the tumor of any of these kinds with a phosphoinositide 3-kinase (PI3K) inhibitor. In some embodiments, the subjects have been previously treated for the tumor of any of these kinds with a poly-ADP ribose polymerase (PARP) inhibitor. In some embodiments, the subjects have been previously treated for the tumor of any of these kinds with bevacizumab. In some embodiments, the subjects have been previously treated for the tumor of any of these kinds with a CDK4/6 inhibitor. In some embodiments, the subjects have been previously treated for the tumor of any of these kinds with a hormonally-directed therapy. The treatment can be applied to patients having tumors of any of these kinds that are unresectable. In some embodiments, the subject has not had a history of another malignancy within 3 years before the first dose of the antibodies or antibody-drug conjugates of the invention. In some embodiments, the subject does not have any evidence of residual disease from a previously diagnosed malignancy at the time of the first dose of the antibodies or antibody-drug conjugates of the invention. In some embodiments, the subject does not have known central nervous system metastases. In some embodiments, the subject does not have carcinomatous meningitis. In some embodiments, the subject has not previously received treatment with an MMAE-containing agent or an agent targeting integrin beta-6.

anti-αvβ6 antibodies of the present invention, such as humanized antibodies, alone or as conjugates thereof, are administered in an effective regime meaning a dosage, route of administration and frequency of administration that delays the onset, reduces the severity, inhibits further deterioration, and/or ameliorates at least one sign or symptom of cancer. If a patient is already suffering from cancer, the regime can be referred to as a therapeutically effective regime. If the patient is at elevated risk of the caner relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

Exemplary dosages for an anti-αvβ6 monoclonal antibody or antibody-drug conjugate described herein are 0.1 mg/kg to 50 mg/kg of the subject's body weight, more typically 1 mg/kg to 30 mg/kg, 1 mg/kg to 20 mg/kg, 1 mg/kg to 15 mg/kg, 1 mg/kg to 12 mg/kg, or 1 mg/kg to 10 mg/kg 1, or 2 mg/kg to 30 mg/kg, 2 mg/kg to 20 mg/kg, 2 mg/kg to 15 mg/kg, 2 mg/kg to 12 mg/kg, or 2 mg/kg to 10 mg/kg, or 3 mg/kg to 30 mg/kg, 3 mg/kg to 20 mg/kg, 3 mg/kg to 15 mg/kg, 3 mg/kg to 12 mg/kg, or 3 mg/kg to 10 mg/kg. In some embodiments, the subject's body weight is the subject's ideal body weight (IBW). In some embodiments, the subject's body weight is the subject's adjusted ideal body weight (AIBW). Exemplary dosages for a monoclonal antibody or antibody drug conjugates thereof are 1 mg/kg to 7.5 mg/kg, or 2 mg/kg to 7.5 mg/kg or 3 mg/kg to 7.5 mg/kg of the subject's body weight, or 0.1-20, or 0.5-5 mg/kg body weight (e.g., 0.5, 1, 2, 3, 4, 5, 6, 7, 8, 9 or 10 mg/kg) or 10-1500 or 200-1500 mg as a fixed dosage. In some embodiments, dose is 0.8 mg/kg. In some embodiments, the dose is 1.0 mg/kg. In some embodiments, the dose is 1.2 mg/kg. In some embodiments, the dose is 1.25 mg/kg. In some embodiments, the dose is 1.5 mg/kg. In some embodiments, the dose is 1.8 mg/kg. In some embodiments, the dose is 2.4 mg/kg. In some methods, the patient is administered a dose of at least 0.8 mg/kg, at least 1.0 mg/kg. at least 1.2 mg/kg, at least 1.25 mg/kg, at least 1.5 mg/kg, at least 1.8 mg/kg, or at least 2.4 mg/kg, administered once every week or greater. In some methods, the patient is administered a dose of at least 0.8 mg/kg, at least 1.0 mg/kg. at least 1.2 mg/kg, at least 1.25 mg/kg, at least 1.5 mg/kg, at least 1.8 mg/kg, or at least 2.4 mg/kg, administered once every week. In some methods, the patient is administered a dose of at least 1.5 mg/kg or at least 1.8 mg/kg administered once every two weeks or greater. In some methods, the patient is administered a dose of at least 1.2 mg/kg or at least 1.25 mg/kg administered twice every three weeks. In some methods, the patient is administered a dose of 1.25 mg/kg administered twice every three weeks. In some methods, the patient is administered a dose of 1.5 mg/kg administered twice every three weeks. In some methods, the patient is administered a dose of 1.8 mg/kg administered once every two weeks. In some methods, the patient is administered a dose of 1.5 mg/kg administered once every two weeks. In some methods, the patient is administered a dose of at least 1.5 mg/kg, at least 2 mg/kg or at least 3 mg/kg, administered once every three weeks or greater. The dosage depends on the frequency of administration, condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Administration can also be localized directly into a tumor. Administration into the systemic circulation by intravenous or subcutaneous administration is preferred. Intravenous administration can be, for example, by infusion over a period such as 30-90 min or by a single bolus injection. In some embodiments, the antibodies or antibody-drug conjugates of the invention are administered by intravenous infusion.

The frequency of administration depends on the half-life of the antibody or conjugate in the circulation, the condition of the patient and the route of administration among other factors. The frequency of administration of an anti-αvβ6 antibody or antibody-drug conjugate described herein can be daily, weekly, once every two weeks, twice every three weeks, once every three weeks, monthly, quarterly, or at irregular intervals in response to changes in the patient's condition or progression of the cancer being treated. In some embodiments, the frequency of administration is about weekly. In some embodiments, the weekly administration is on about Days 1, 8, and 15 of a 21-day cycle. In some embodiments, the frequency of administration is once about every two weeks. In some embodiments, the frequency of administration is twice about every three weeks. In some embodiments, the frequency of administration is on about Days 1 and 8 of a 21-day cycle. In some embodiments, the frequency of administration is once about every three weeks. In some embodiments, the frequency of administration is weekly. In some embodiments, the weekly administration is on Days 1, 8, and 15 of a 21-day cycle. In some embodiments, the frequency of administration is once every two weeks. In some embodiments, the frequency of administration is on Days 1 and 8 of a 21-day cycle. In some embodiments, the frequency of administration is twice every three weeks. In some embodiments, the frequency of administration is once every three weeks. For subcutaneous administration, an exemplary dosing frequency is daily to monthly, although more or less frequent dosing is also possible. An exemplary frequency for intravenous administration is between twice a week and quarterly over a continuous course of treatment, although more or less frequent dosing is also possible. Other exemplary frequencies for intravenous administration are between weekly or three out of every four weeks over a continuous course of treatment, although more or less frequent dosing is also possible. Other exemplary frequencies for intravenous administration are once every two weeks or once every three weeks over a continuous course of treatment, although more or less frequent dosing is also possible. In some embodiments, the dose is administered is based on the subject's body weight. In some embodiments, the subject's body weight is the subject's ideal body weight (IBW). In some embodiments, the subject's body weight is the subjects adjusted ideal body weight (AIBW). In some embodiments, the dose is 0.7 mg/kg and is administered once about every 1 week. In some embodiments, the dose is 0.7 mg/kg and is administered once about every 2 weeks. In some embodiments, the dose is 0.7 mg/kg and is administered twice about every 3 weeks. In some embodiments, the dose is 0.7 mg/kg and is administered once about every 3 weeks. In some embodiments, the dose is 0.8 mg/kg and is administered once about every 1 week. In some embodiments, the dose is 0.8 mg/kg and is administered once about every 2 weeks. In some embodiments, the dose is 0.8 mg/kg and is administered twice about every 3 weeks. In some embodiments, the dose is 0.8 mg/kg and is administered once about every 3 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered twice about every 3 weeks. In some embodiments, the dose is 0.9 mg/kg and is administered once about every 3 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered twice about every 3 weeks. In some embodiments, the dose is 1.0 mg/kg and is administered once about every 3 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered twice about every 3 weeks. In some embodiments, the dose is 1.1 mg/kg and is administered once about every 3 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered twice about every 3 weeks. In some embodiments, the dose is 1.2 mg/kg and is administered once about every 3 weeks. In some embodiments, the dose is 1.25 mg/kg and is administered once about every 1 week. In some embodiments, the dose is 1.25 mg/kg and is administered once about every 2 weeks. In some embodiments, the dose is 1.25 mg/kg and is administered twice about every 3 weeks. In some embodiments, the dose is 1.25 mg/kg and is administered once about every 3 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered twice about every 3 weeks. In some embodiments, the dose is 1.3 mg/kg and is administered once about every 3 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered twice about every 3 weeks. In some embodiments, the dose is 1.4 mg/kg and is administered once about every 3 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered twice about every 3 weeks. In some embodiments, the dose is 1.5 mg/kg and is administered once about every 3 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered twice about every 3 weeks. In some embodiments, the dose is 1.6 mg/kg and is administered once about every 3 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered twice about every 3 weeks. In some embodiments, the dose is 1.7 mg/kg and is administered once about every 3 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered twice about every 3 weeks. In some embodiments, the dose is 1.8 mg/kg and is administered once about every 3 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered twice about every 3 weeks. In some embodiments, the dose is 1.9 mg/kg and is administered once about every 3 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered twice about every 3 weeks. In some embodiments, the dose is 2.0 mg/kg and is administered once about every 3 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered twice about every 3 weeks. In some embodiments, the dose is 2.1 mg/kg and is administered once about every 3 weeks. In some embodiments, the dose is 2.2 mg/kg and is administered once about every 1 week. In some embodiments, the dose is 2.2 mg/kg and is administered once about every 2 weeks. In some embodiments, the dose is 2.2 mg/kg and is administered twice about every 3 weeks. In some embodiments, the dose is 2.2 mg/kg and is administered once about every 3 weeks. In some embodiments, the dose is 2.3 mg/kg and is administered once about every 1 week. In some embodiments, the dose is 2.3 mg/kg and is administered once about every 2 weeks. In some embodiments, the dose is 2.3 mg/kg and is administered twice about every 3 weeks. In some embodiments, the dose is 2.3 mg/kg and is administered once about every 3 weeks. In some embodiments, the dose is 2.4 mg/kg and is administered once about every 1 week. In some embodiments, the dose is 2.4 mg/kg and is administered once about every 2 weeks. In some embodiments, the dose is 2.4 mg/kg and is administered twice about every 3 weeks. In some embodiments, the dose is 2.4 mg/kg and is administered once about every 3 weeks. In some embodiments, the dose is 2.5 mg/kg and is administered once about every 1 week. In some embodiments, the dose is 2.5 mg/kg and is administered once about every 2 weeks. In some embodiments, the dose is 2.5 mg/kg and is administered twice about every 3 weeks. In some embodiments, the dose is 2.5 mg/kg and is administered once about every 3 weeks.

The number of dosages administered depends on the nature of the cancer (e.g., whether presenting acute or chronic symptoms) and the response of the disorder to the treatment. For acute disorders or acute exacerbations of a chronic disorder between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the patient.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, antibodies can be in lyophilized form for constitution with a suitable vehicle, e.g., sterile pyrogen-free water, before use. The concentration of antibody in a liquid formulation can be e.g., 1-100 mg/ml, such as 10 mg/ml.

Treatment with the anti-αvβ6 antibodies or antibody-drug conjugates described herein can be combined with one or more additional treatments, such as chemotherapy, radiation, stem cell treatment, surgery other treatments effective against the disorder being treated. Useful classes of other agents that can be administered with antibodies and antibody-drug conjugates to αvβ6 as described herein include, for example, antibodies to other receptors expressed on cancerous cells, antitubulin agents (e.g., auristatins), DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, and the like. In some embodiments, the one or more additional treatments is the administration of a checkpoint inhibitor. In the additional therapeutic agent is a PD-1 inhibitor or a PD-L1 inhibitor. In some embodiments, the PD-1 inhibitor is an anti-PD-1 antibody. In some embodiments, the anti-PD-1 antibody is AMP-224, CT-011, cemiplimab, camrelizumab, sintilimab, tislelizumab, TSR-042, PDR001, toripalimab, BGB-A317, nivolumab (also known as ONO-4538, BMS-936558, or MDX1106), pembrolizumab (also known as MK-3475, SCH 900475, or lambrolizumab), sasanlimab (also known as PF-06801591), dostarlimab, or a biosimilar of any of these antibodies. In some embodiments, the PD-L1 inhibitor is an anti-PD-L1 antibody. In some embodiments, the anti-PD-L1 antibody is MEDI4736 (also known as durvalumab or IMFINZI®), BMS-936559 (also known as MDX-1105-01), atezolizumab (also known as MPDL3280A, and Tecentriq®), avelumab (also known as BAVENCIO®), or a biosimilar of any of these antibodies. In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. In yet another embodiment, the anti-PD-1 antibody is AMP-224, a fusion protein. In another embodiment, the anti-PD-1 antibody is BGB-A317. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab. In another embodiment, the anti-PD-1 antibody is camrelizumab. In a further embodiment, the anti-PD-1 antibody is sintilimab. In some embodiments, the anti-PD-1 antibody is tislelizumab. In certain embodiments, the anti-PD-1 antibody is TSR-042. In yet another embodiment, the anti-PD-1 antibody is PDR001. In yet another embodiment, the anti-PD-1 antibody is toripalimab. In yet another embodiment, the anti-PD-1 antibody is sasanlimab. In yet another embodiment, the anti-PD-1 antibody is dostarlimab. In some embodiments, the anti-PD-L1 antibody is MEDI4736 (also known as durvalumab or IMFINZI®), BMS-936559 (also known as MDX-1105-01), atezolizumab (also known as MPDL3280A, and Tecentriq®), or avelumab (also known as BAVENCIO®). In one embodiment, the anti-PD-L1 antibody is MEDI4736 (durvalumab). In another embodiment, the anti-PD-L1 antibody is BMS-936559. In yet another embodiment, the PD-L1 inhibitor is atezolizumab. In a further embodiment, the PD-L1 inhibitor is avelumab.

In some embodiments, the one or more additional treatments are administered simultaneously with the anti-αvβ6 antibodies or antibody-drug conjugates described herein. In some embodiments, the one or more additional treatments and the anti-αvβ6 antibodies or antibody-drug conjugates described herein are administered sequentially. In some embodiments, simultaneous means that the anti-αvβ6 antibodies or antibody-drug conjugates described herein and the one or more additional treatments are administered to the subject less than one hour apart, such as less than about 30 minutes apart, less than about 15 minutes apart, less than about 10 minutes apart or less than about 5 minutes apart. In some embodiments, sequential administration means that the anti-αvβ6 antibodies or antibody-drug conjugates described herein and the one or more additional treatments are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart, at least 4 weeks apart, at least 6 weeks apart, at least 2 months apart, at least 3 months apart, at least 4 months apart, at least 5 months apart, or at least 6 months apart.

In some embodiments, the one or more additional treatments is the administration of a chemotherapeutic agent. In some embodiments, the one or more additional treatments is the administration of a platinum-based agent. In some embodiments, the platinum-based agent is carboplatin or cisplatin. In some embodiments, the platinum-based agent is carboplatin. In some embodiments, the platinum-based agent is cisplatin.

In some embodiments, the one or more additional treatments is the administration of a platinum-based agent and a checkpoint inhibitor. In some embodiments, the platinum-based agent is carboplatin or cisplatin. In some embodiments, the platinum-based agent is carboplatin. In some embodiments, the platinum-based agent is cisplatin. In some embodiments, the checkpoint inhibitor is pembrolizumab, or a biosimilar thereof. In some embodiments, the checkpoint inhibitor is pembrolizumab.

In some embodiments, the one or more additional treatments is the administration of pembrolizumab, or a biosimilar thereof. In some embodiments, the pembrolizumab or biosimilar thereof is administered at a dose of about 200 mg. In some embodiments, the pembrolizumab or biosimilar thereof is administered at a dose of 200 mg. In some embodiments, the pembrolizumab or biosimilar thereof is administered at a dose of about 400 mg. In some embodiments, the pembrolizumab or biosimilar thereof is administered at a dose of 400 mg. In some embodiments, the pembrolizumab or biosimilar thereof is administered once about every 3 weeks. In some embodiments, the pembrolizumab or biosimilar thereof is administered once every 3 weeks. In some embodiments, the pembrolizumab or biosimilar thereof is administered once about every 6 weeks. In some embodiments, the pembrolizumab or biosimilar thereof is administered once every 6 weeks. In some embodiments, pembrolizumab or biosimilar thereof is administered at a dose of about 200 mg once about every 3 weeks. In some embodiments, pembrolizumab or biosimilar thereof is administered at a dose of 200 mg once every 3 weeks. In some embodiments, pembrolizumab or biosimilar thereof is administered at a dose of about 400 mg once about every 6 weeks. In some embodiments, pembrolizumab or biosimilar thereof is administered at a dose of 400 mg once every 6 weeks. In some embodiments, the route of administration of the pembrolizumab or biosimilar thereof is intravenous. In some embodiments, the first dose of the anti-αvβ6 antibody or antibody drug conjugate is administered to the subject prior to the administration of the first dose of pembrolizumab or biosimilar thereof. In some embodiments, the first dose of the anti-αvβ6 antibody or antibody drug conjugate is administered to the subject at least 1 day, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months or at least 6 months prior to the administration of the first dose of pembrolizumab or biosimilar thereof. In some embodiments, the first dose of the anti-αvβ6 antibody or antibody drug conjugate is administered to the subject at least 1 day prior to the administration of the first dose of pembrolizumab or biosimilar thereof. In some embodiments, the first dose of the anti-αvβ6 antibody or antibody drug conjugate is administered to the subject at least 2 days prior to the administration of the first dose of pembrolizumab or biosimilar thereof. In some embodiments, the first dose of the anti-αvβ6 antibody or antibody drug conjugate is administered to the subject at least 3 days prior to the administration of the first dose of pembrolizumab or biosimilar thereof. In some embodiments, the first dose of the anti-αvβ6 antibody or antibody drug conjugate is administered to the subject at least 4 days prior to the administration of the first dose of pembrolizumab or biosimilar thereof. In some embodiments, the first dose of the anti-αvβ6 antibody or antibody drug conjugate is administered to the subject at least 5 days prior to the administration of the first dose of pembrolizumab or biosimilar thereof. In some embodiments, the first dose of the anti-αvβ6 antibody or antibody drug conjugate is administered to the subject at least 6 days prior to the administration of the first dose of pembrolizumab or biosimilar thereof. In some embodiments, the first dose of the anti-αvβ6 antibody or antibody drug conjugate is administered to the subject at least 7 days prior to the administration of the first dose of pembrolizumab or biosimilar thereof. In some embodiments, the first dose of the anti-αvβ6 antibody or antibody drug conjugate is administered to the subject at least 1 week prior to the administration of the first dose of pembrolizumab or biosimilar thereof. In some embodiments, the first dose of the anti-αvβ6 antibody or antibody drug conjugate is administered to the subject at least 2 weeks prior to the administration of the first dose of pembrolizumab or biosimilar thereof. In some embodiments, the first dose of the anti-αvβ6 antibody or antibody drug conjugate is administered to the subject at least 3 weeks prior to the administration of the first dose of pembrolizumab or biosimilar thereof. In some embodiments, the first dose of the anti-αvβ6 antibody or antibody drug conjugate is administered to the subject at least 4 weeks prior to the administration of the first dose of pembrolizumab or biosimilar thereof. In some embodiments, the first dose of the anti-αvβ6 antibody or antibody drug conjugate is administered to the subject at least 2 months prior to the administration of the first dose of pembrolizumab or biosimilar thereof. In some embodiments, the first dose of the anti-αvβ6 antibody or antibody drug conjugate is administered to the subject at least 3 months prior to the administration of the first dose of pembrolizumab or biosimilar thereof. In some embodiments, the first dose of the anti-αvβ6 antibody or antibody drug conjugate is administered to the subject at least 4 months prior to the administration of the first dose of pembrolizumab or biosimilar thereof. In some embodiments, the first dose of the anti-αvβ6 antibody or antibody drug conjugate is administered to the subject at least 5 months prior to the administration of the first dose of pembrolizumab or biosimilar thereof. In some embodiments, the first dose of the anti-αvβ6 antibody or antibody drug conjugate is administered to the subject at least 6 months prior to the administration of the first dose of pembrolizumab or biosimilar thereof.

Treatment with the anti-αvβ6 antibody or antibody-drug conjugate, optionally in combination with any of the other agents or regimes described above, can increase the median progression-free survival or overall survival time of patients with solid tumors (e.g., non-small cell lung cancer (NSCLC) (squamous and adeno), head and neck cancer (including head and neck squamous carcinoma), esophageal cancer (including esophageal squamous cell carcinoma), breast cancer (including breast invasive carcinoma), HER2-breast cancer, ovarian cancer (including high grade serous ovarian cancer (HGSOC), bladder cancer (including urothelial carcinoma), skin cancer (including squamous cell carcinoma, or SCC), renal cancer (including renal clear cell, renal papillary cell, and kidney chromophobe), cervical cancer, gastric cancer, prostate cancer (including prostate adenocarcinoma), endometrial cancer (including uterine carcinosarcoma and uterine corpus endometrial), rectum adenocarcinoma, thyroid carcinoma, colon adenocarcinoma, stomach adenocarcinoma, and pancreatic cancer (including pancreatic adenocarcinoma)), especially when relapsed or refractory, by at least 30% or 40% but preferably 50%, 60% to 70% or even 100% or longer, compared to the same treatment (e.g., chemotherapy) but without an anti-αvβ6 antibody alone or as a conjugate. In addition, or alternatively, treatment (e.g., standard chemotherapy) including the anti-αvβ6 antibody alone or as a conjugate can increase the complete response rate, partial response rate, or objective response rate (complete+partial) of patients with tumors by at least 30% or 40% but preferably 50%, 60% to 70% or even 100% compared to the same treatment (e.g., chemotherapy) but without the anti-αvβ6 antibody alone or as a conjugate.

In one aspect, a method of treating a solid tumor with an anti-αvβ6 antibody or antibody-drug conjugate described herein results in an improvement in one or more therapeutic effects in the subject after administration of the antibody or antibody-drug conjugate described herein relative to a baseline. In some embodiments, the one or more therapeutic effects is the size of the tumor derived from the cancer, the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with an anti-αvβ6 antibody or antibody-drug conjugate described herein may include the following criteria (RECIST Criteria 1.1):

| | Category | Criteria |
|---|---|---|
| Based on | Complete | Disappearance of all target lesions. Any pathological |
| target lesions | Response (CR) | lymph nodes must have reduction in short axis to <10 mm. |
| | Partial Response | ≥30% decrease in the sum of the longest diameter |
| | (PR) | (LD) of target lesions, taking as reference the baseline |
| | | sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on non- | CR | Disappearance of all non-target lesions and |
| target lesions | | normalization of tumor marker level. All lymph nodes |
| | | must be non-pathological in size (< 10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

In one embodiment of the methods or uses or product for uses provided herein, the effectiveness of treatment with an anti-αvβ6 antibody or antibody-drug conjugate described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an anti-αvβ6 antibody or antibody-drug conjugate described herein is assessed by measuring the size of a tumor derived from the cancer. In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antibody-drug conjugate described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or antibody-drug conjugate described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by ultrasound.

In one embodiment of the methods or uses or product for uses provided described herein, response to treatment with an anti-αvβ6 antibody or antibody-drug conjugate described herein promotes regression of a tumor derived from the cancer. In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antibody-drug conjugate described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or antibody-drug conjugate described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In one embodiment, regression of a tumor is determined by measuring the size of the tumor by positron emission tomography (PET). In one embodiment, regression of a tumor is determined by measuring the size of the tumor by ultrasound.

In one embodiment of the methods or uses or product for uses described herein, response to treatment with an anti-αvβ6 antibody or antibody-drug conjugate described herein is assessed by measuring the time of progression free survival after administration of the antibody or antibody-drug conjugate described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antibody-drug conjugate described herein. In some embodiments, the subject exhibits progression-free survival of at least about 6 months after administration of the antibody or antibody-drug conjugate described herein. In some embodiments, the subject exhibits progression-free survival of at least about one year after administration of the antibody or antibody-drug conjugate described herein. In some embodiments, the subject exhibits progression-free survival of at least about two years after administration of the antibody or antibody-drug conjugate described herein. In some embodiments, the subject exhibits progression-free survival of at least about three years after administration of the antibody or antibody-drug conjugate described herein. In some embodiments, the subject exhibits progression-free survival of at least about four years after administration of the antibody or antibody-drug conjugate described herein. In some embodiments, the subject exhibits progression-free survival of at least about five years after administration of the antibody or antibody-drug conjugate described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antibody-drug conjugate described herein. In some embodiments, the subject exhibits progression-free survival of at least 6 months after administration of the antibody or antibody-drug conjugate described herein. In some embodiments, the subject exhibits progression-free survival of at least one year after administration of the antibody or antibody-drug conjugate described herein. In some embodiments, the subject exhibits progression-free survival of at least two years after administration of the antibody or antibody-drug conjugate described herein. In some embodiments, the subject exhibits progression-free survival of at least three years after administration of the antibody or antibody-drug conjugate described herein. In some embodiments, the subject exhibits progression-free survival of at least four years after administration of the antibody or antibody-drug conjugate described herein. In some embodiments, the subject exhibits progression-free survival of at least five years after administration of the antibody or antibody-drug conjugate described herein.

In one embodiment of the methods or uses or product for uses described herein, response to treatment with an anti-αvβ6 antibody or antibody-drug conjugate described herein is assessed by measuring the time of overall survival after administration of the antibody or antibody-drug conjugate described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antibody-drug conjugate described herein. In some embodiments, the subject exhibits overall survival of at least about 6 months after administration of the antibody or antibody-drug conjugate described herein. In some embodiments, the subject exhibits overall survival of at least about one year after administration of the antibody or antibody-drug conjugate described herein. In some embodiments, the subject exhibits overall survival of at least about two years after administration of the antibody or antibody-drug conjugate described herein. In some embodiments, the subject exhibits overall survival of at least about three years after administration of the antibody or antibody-drug conjugate described herein. In some embodiments, the subject exhibits overall survival of at least about four years after administration of the antibody or antibody-drug conjugate described herein. In some embodiments, the subject exhibits overall survival of at least about five years after administration of the antibody or antibody-drug conjugate described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antibody-drug conjugate described herein. In some embodiments, the subject exhibits overall survival of at least 6 months after administration of the antibody or antibody-drug conjugate described herein. In some embodiments, the subject exhibits overall survival of at least one year after administration of the antibody or antibody-drug conjugate described herein. In some embodiments, the subject exhibits overall survival of at least two years after administration of the antibody or antibody-drug conjugate described herein. In some embodiments, the subject exhibits overall survival of at least three years after administration of the antibody or antibody-drug conjugate described herein. In some embodiments, the subject exhibits overall survival of at least four years after administration of the antibody or antibody-drug conjugate described herein. In some embodiments, the subject exhibits overall survival of at least five years after administration of the antibody or antibody-drug conjugate described herein.

In one embodiment of the methods or uses or product for uses described herein, response to treatment with an anti-αvβ6 antibody or antibody-drug conjugate described herein is assessed by measuring the duration of response to the antibody or antibody-drug conjugate described herein after administration of the antibody or antibody-drug conjugate described herein. In some embodiments, the duration of response to the antibody or antibody-drug conjugate described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody or antibody-drug conjugate described herein. In some embodiments, the duration of response to the antibody or antibody-drug conjugate described herein is at least about 6 months after administration of the antibody or antibody-drug conjugate described herein. In some embodiments, the duration of response to the antibody or antibody-drug conjugate described herein is at least about one year after administration of the antibody or antibody-drug conjugate described herein. In some embodiments, the duration of response to the antibody or antibody-drug conjugate described herein is at least about two years after administration of the antibody or antibody-drug conjugate described herein. In some embodiments, the duration of response to the antibody or antibody-drug conjugate described herein is at least about three years after administration of the antibody or antibody-drug conjugate described herein. In some embodiments, the duration of response to the antibody or antibody-drug conjugate described herein is at least about four years after administration of the antibody or antibody-drug conjugate described herein. In some embodiments, the duration of response to the antibody or antibody-drug conjugate described herein is at least about five years after administration of the antibody or antibody-drug conjugate described herein. In some embodiments, the duration of response to the antibody or antibody-drug conjugate described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the antibody or antibody-drug conjugate described herein. In some embodiments, the duration of response to the antibody or antibody-drug conjugate described herein is at least 6 months after administration of the antibody or antibody-drug conjugate described herein. In some embodiments, the duration of response to the antibody or antibody-drug conjugate described herein is at least one year after administration of the antibody or antibody-drug conjugate described herein. In some embodiments, the duration of response to the antibody or antibody-drug conjugate described herein is at least two years after administration of the antibody or antibody-drug conjugate described herein. In some embodiments, the duration of response to the antibody or antibody-drug conjugate described herein is at least three years after administration of the antibody or antibody-drug conjugate described herein. In some embodiments, the duration of response to the antibody or antibody-drug conjugate described herein is at least four years after administration of the antibody or antibody-drug conjugate described herein. In some embodiments, the duration of response to the antibody or antibody-drug conjugate described herein is at least five years after administration of the antibody or antibody-drug conjugate described herein.

Typically, in a clinical trial (e.g., a phase I, phase II, phase II/III or phase III trial), the aforementioned increases in median progression-free survival and/or overall survival and/or response rate and/or duration of response of the patients treated with standard therapy plus the anti-αvβ6 antibody alone or as conjugate, relative to the control group of patients receiving standard therapy alone (or plus placebo), are statistically significant, for example at the p=0.05 or 0.01 or even 0.001 level. The complete and partial response rates are determined by objective criteria commonly used in clinical trials for cancer, e.g., as listed or accepted by the National Cancer Institute and/or Food and Drug Administration.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells from the subject express PD-L1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the cancer cells from the subject express PD-L1. In some of any of the embodiments herein, the subject's tumor expresses PD-L1 with a tumor proportion score (TPS)≥1%. In some of any of the embodiments herein, the subject's tumor expresses PD-L1 with a TPS ≥5%. In some of any of the embodiments herein, the subject's tumor expresses PD-L1 with a TPS ≥10%. In some of any of the embodiments herein, the subject's tumor expresses PD-L1 with a TPS ≥15%. In some of any of the embodiments herein, the subject's tumor expresses PD-L1 with a TPS ≥20%. In some of any of the embodiments herein, the subject's tumor expresses PD-L1 with a TPS ≥25%. In some of any of the embodiments herein, the subject's tumor expresses PD-L1 with a TPS ≥30%. In some of any of the embodiments herein, the subject's tumor expresses PD-L1 with a TPS ≥35%. In some of any of the embodiments herein, the subject's tumor expresses PD-L1 with a TPS ≥40%. In some of any of the embodiments herein, the subject's tumor expresses PD-L1 with a TPS ≥45%. In some of embodiments herein, the subject's tumor has high PD-L1 expression with TPS≥50%. In some embodiments herein, the subject's tumor expresses PD-L1 with a combined positive score (CPS) ≥1%. See US 2017/0285037. In some embodiments herein, the subject's tumor expresses PD-L1 with a CPS ≥5%. In some embodiments herein, the subject's tumor expresses PD-L1 with a CPS ≥10%. In some embodiments herein, the subject's tumor expresses PD-L1 with a CPS ≥15%. In some embodiments herein, the subject's tumor expresses PD-L1 with a CPS ≥20%. In some embodiments herein, the subject's tumor expresses PD-L1 with a CPS ≥25%. In some embodiments herein, the subject's tumor expresses PD-L1 with a CPS ≥30%. In some embodiments herein, the subject's tumor expresses PD-L1 with a CPS ≥35%. In some embodiments herein, the subject's tumor expresses PD-L1 with a CPS ≥40%. In some embodiments herein, the subject's tumor expresses PD-L1 with a CPS ≥45%. In some embodiments herein, the subject's tumor expresses PD-L1 with a CPS ≥50%. In some embodiments, the percentage of cells that express PD-L1 is determined using immunohistochemistry (IHC). In some embodiments, the percentage of cells that express PD-L1 is determined using flow cytometry. In some embodiments, the percentage of cells that express PD-L1 is determined using an enzyme-linked immunosorbent assay (ELISA). In some embodiments, the percentage of cells that express PD-L1 is assessed prior to the methods or uses or product for uses described herein.

### IX. Articles of Manufacture and Kits

In another aspect, an article of manufacture or kit is provided which comprises an anti-αvβ6 antibody or anti-αvβ6 antibody-drug conjugate described herein. The article of manufacture or kit may further comprise instructions for use of the anti-αvβ6 antibody or anti-αvβ6 antibody-drug conjugate described herein in the methods of the invention. Thus, in certain embodiments, the article of manufacture or kit comprises instructions for the use of an anti-αvβ6 antibody or anti-αvβ6 antibody-drug conjugate described herein in methods for treating cancer (e.g., non-small cell lung cancer (NSCLC) (squamous and adeno), head and neck cancer (including head and neck squamous carcinoma), esophageal cancer, breast cancer (including breast invasive carcinoma), ovarian cancer, bladder cancer (including urothelial carcinoma), skin cancer (squamous cell carcinoma, or SCC), renal cancer (including renal clear cell, renal papillary cell, and kidney chromophobe), cervical cancer, gastric cancer, prostate cancer (including prostate adenocarcinoma), endometrial cancer (including uterine carcinosarcoma and uterine corpus endometrial), rectum adenocarcinoma, thyroid carcinoma, colon adenocarcinoma, stomach adenocarcinoma, and pancreatic cancer (including pancreatic adenocarcinoma)) in a subject comprising administering to the subject an effective amount of an anti-αvβ6 antibody or anti-αvβ6 antibody-drug conjugate described herein. In some embodiments, the cancer is NSCLC. In some embodiments, the NSCLC is a squamous cell carcinoma. In some embodiments, the NSCLC is a non-squamous cell carcinoma. In some embodiments, the NSCLC is an adenocarcinoma. In some embodiments, the NSCLC does not harbor known mutations/alterations that confer eligibility for approved targeted therapies (e.g., epidermal growth factor receptor (EGFR), anaplastic lymphoma kinase (ALK), etc.). In some embodiments, the NSCLC has one or more known mutations/alterations that confer eligibility for approved targeted therapies (e.g., epidermal growth factor receptor (EGFR), anaplastic lymphoma kinase (ALK), etc.) In some embodiments, the NSCLC has a known EGFR mutation.

In some embodiments, the NSCLC has a known ALK mutation. In some embodiments, the cancer is head and neck cancer. In some embodiments, the head and neck cancer is a squamous cell carcinoma. In some embodiments, the cancer is esophageal cancer. In some embodiments, the esophageal cancer is esophageal squamous cell carcinoma. In some embodiments, the cancer is breast cancer. In some embodiments, the breast cancer is a HER2-negative breast cancer. In some embodiments, the breast cancer is breast invasive carcinoma. In some embodiments, the cancer is ovarian cancer. In some embodiments, the ovarian cancer is high grade serous epithelial ovarian cancer. In some embodiments, the cancer is primary peritoneal cancer. In some embodiments, the cancer is fallopian tube cancer. In some embodiments, cancer is renal cancer. In some embodiments, the renal cancer is renal clear cell carcinoma. In some embodiments, the renal cancer is renal papillary cell carcinoma. In some embodiments, the renal cancer is kidney chromophobe carcinoma. In some embodiments, the cancer is endometrial cancer. In some embodiments, the endometrial cancer is uterine carcinosarcoma. In some embodiments, the endometrial cancer is uterine corpus endometrial carcinoma. In some embodiments, the cancer is stomach cancer. In some embodiments, the stomach cancer is stomach adenocarcinoma. In some embodiments, the cancer is bladder cancer. In some embodiments, the bladder cancer is urothelial carcinoma. In some embodiments, the cancer is skin cancer. In some embodiments, the skin cancer is cutaneous squamous cell carcinoma. In some embodiments, the cancer is prostate cancer. In some embodiments, the prostate cancer is prostate adenocarcinoma. In some embodiments, the cancer is rectum adenocarcinoma. In some embodiments, the cancer is thyroid carcinoma. In some embodiments, the cancer is colon adenocarcinoma. In some embodiments, the cancer is cervical cancer. In some embodiments, the cancer is gastric cancer. In some embodiments, the cancer is pancreatic cancer. In some embodiments, the pancreatic cancer is exocrine pancreatic adenocarcinoma. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cancer in a subject (e.g., non-small cell lung cancer (NSCLC) (squamous and adeno), head and neck cancer (including head and neck squamous carcinoma), esophageal cancer, breast cancer (including breast invasive carcinoma), ovarian cancer, bladder cancer (including urothelial carcinoma), skin cancer (squamous cell carcinoma, or SCC), renal cancer (including renal clear cell, renal papillary cell, and kidney chromophobe), cervical cancer, gastric cancer, prostate cancer (including prostate adenocarcinoma), endometrial cancer (including uterine carcinosarcoma and uterine corpus endometrial), rectum adenocarcinoma, thyroid carcinoma, colon adenocarcinoma, stomach adenocarcinoma, and pancreatic cancer (including pancreatic adenocarcinoma)). The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-αvβ6 antibody or anti-αvβ6 antibody-drug conjugate is a first medicament, and which article or kit further comprises instructions on the label or package insert for treating the subject with the second medicament, in an effective amount. In some embodiments, the second medicament is for eliminating or reducing the severity of one or more adverse events.

In some embodiments, the anti-αvβ6 antibody or anti-αvβ6 antibody-drug conjugate is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

### X. Other Applications

The anti-αvβ6 antibodies described herein, such as humanized anti-αvβ6, antibodies can be used for detecting αvβ6 in the context of clinical diagnosis or treatment or in research. Expression of αvβ6 on a cancer provides an indication that the cancer is amenable to treatment with the antibodies of the present invention. The antibodies can also be sold as research reagents for laboratory research in detecting cells bearing αvβ6 and their response to various stimuli. In such uses, monoclonal antibodies can be labeled with fluorescent molecules, spin-labeled molecules, enzymes or radioisotypes, and can be provided in the form of kit with all the necessary reagents to perform the assay for αvβ6. The antibodies described herein, can be used to detect αvβ6 protein expression and determine whether a cancer is amenable to treatment with αvβ6 ADCs.

All patent filings, website, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1: A Phase 1 Study of SGN-B6A in Advanced Solid Tumors

SGN-B6A is an antibody-drug conjugate (ADC) that targets integrin beta-6 (AVB6), a cell surface adhesion protein that has high prevalence in several types of cancer. SGN-B6A is comprised of a humanized IgG1 anti-integrin beta-6 monoclonal antibody (h2A2) conjugated to the tubulin-disrupting antimitotic drug monomethyl auristatin E (MMAE) via a cleavable peptide linker. Upon binding to integrin beta-6 on the surface of malignant cells, SGN-B6A is designed to internalize and release the cytotoxic MMAE. A summary of clinical and nonclinical data relevant to SGN-B6A and its study in human subjects is provided in the Investigator's Brochure (IB).

Keytruda® (pembrolizumab) is a potent humanized IgG4 monoclonal antibody (mAb) with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Pembrolizumab has an acceptable preclinical safety profile and is both approved and in clinical development as an intravenous (IV) immunotherapy for advanced malignancies. Pembrolizumab is indicated for the treatment of patients across several indications. For detailed background, refer to the pembrolizumab Product Information.

This first-in-human (FIH) study is designed to evaluate the safety, tolerability, pharmacokinetics, and antitumor activity of SGN-B6A monotherapy (Parts A and B) and in combination with pembrolizumab (Parts C and D) in adults with select advanced solid tumors.

This is a phase 1, open-label, multicenter study designed to evaluate the safety, tolerability, PK, and antitumor activity of SGN-B6A in adults with select advanced solid tumors. The study will include dose escalation and dose expansion, with multiple disease-specific cohorts and a biology cohort in dose expansion. The biology cohort will be gated on data generated in other parts of the trial and will require additional biopsies. At the completion of dose escalation for a particular schedule, up to 9 disease-specific expansion cohorts and a biology cohort may be activated by the sponsor in consultation with the Safety Monitoring Committee (SMC). Expansion cohorts in Part B will enroll selected tumor types eligible for enrollment in Part A.

### Methods

This study is a phase 1, dose-escalation study in subjects with advanced solid tumors. Primary objectives of Part A are to evaluate the safety and tolerability of SGN-B6A in subjects with advanced solid tumors, to identify the maximum tolerated dose (MTD), and to identity a recommended dose and schedule. SGN-B6A will initially be given by intravenous (IV) infusion on Days 1, 8, and 15 of 21-day cycles. Other dosing regimens may be explored. The dose-escalation portion of the study (Part A) will be conducted using the modified toxicity probability interval (mTPI) method to determine a dose that demonstrates a dose-limiting toxicity (DLT) rate of 25% with a 5% margin. For the dose-escalation portion of the study (Part A), SGN-B6A dose level and administration schedule will be defined by cohort assignment. The dose and schedule for the dose-expansion portion (Part B) will be determined based on evaluation of safety, pharmacokinetics (PK), and pharmacodynamic biomarkers. For the dose-expansion portion (Part B), SGN-B6A at or below MTD and/or recommended dose and schedule as determined in Part A.

Because integrin beta-6 is expressed in several tumor types, SGN-B6A may be active in a wide variety of malignancies. Ten specific tumor types are targeted for initial clinical evaluation during dose escalation in Part A: non-small cell lung cancer (NSCLC), head and neck squamous cell cancer (HNSCC), advanced human epidermal growth factor receptor 2 (HER2)-negative breast cancer, esophageal squamous cell carcinoma (ESCC), high grade serous ovarian cancer (HGSOC), cutaneous squamous cell carcinoma (cSCC), exocrine pancreatic adenocarcinoma, bladder cancer, cervical cancer, and gastric cancer. At the completion of dose escalation for a particular schedule, up to 9 disease-specific expansion cohorts and 1 biology cohort will be activated in Part B to further evaluate safety and preliminary antitumor efficacy of SGN-B6A.

The primary objective of Part C is to evaluate the safety and tolerability of SGN-B6A in combination with pembrolizumab. At the completion of Part C, 2 disease-specific expansion cohorts, NSCLC and HNSCC, will be activated to further evaluate safety and preliminary antitumor activity of SGN-B6A in combination with pembrolizumab (Part D).

The planned dose of pembrolizumab for Part C and Part D of this study is 200 mg Q3W or 400 mg Q6W. Based on the totality of data generated in the pembrolizumab development program, 200 mg Q3W and 400 mg Q6W are appropriate doses of pembrolizumab for adults across all indications. As outlined below, this dose is justified by:
Clinical data from 8 randomized studies in melanoma and NSCLC indications demonstrating flat dose- and exposure-efficacy relationships from 2 mg/kg Q3W to 10 mg/kg every 2 weeks (Q2W) representing an approximate 5- to 7.5-fold exposure range
Population PK analysis showing that both fixed dosing and weight-based dosing provides similar control of PK variability with considerable overlap in the distributions of exposures, supporting suitability of 200 mg Q3W
Clinical data showing meaningful improvement in benefit-risk including overall survival at 200 mg Q3W across multiple indications, and
Pharmacology data showing full target saturation in both systemic circulation (inferred from PK data) and tumor (inferred from physiologically based PK analysis) at 200 mg Q3W.

### Part A—Dose-Escalation Cohorts

The dose-escalation portion of this trial (Part A) will be conducted in approximately 85 subjects using the mTPI method. Part A is designed to evaluate safety and tolerability and to identify the MTD of SGN-B6A. If the MTD is not reached, safety, PK, pharmacodynamic, and biomarker analyses, as well as preliminary antitumor activity, may be used to determine a recommended dose.

The mTPI method uses a Bayesian statistical framework and a beta-binomial hierarchical model to compute the posterior probabilities of 3 intervals that reflect the relative distance between the toxicity rate of each dose level to the target DLT rate. Dosing-decision rules are determined for a target DLT rate of 25% with a 5% margin. The 3 intervals will be (0, 20%), (20%, 30%), and (30%, 100%), and the corresponding dose decision rules would be:
1. Escalate if the current DLT rate is most likely <20%
2. Continue if the current DLT rate is most likely between 20% and 30%
3. De-escalate if the current DLT rate is likely >30%

Dose-finding decisions are shown in Table 1. “E” represents escalating the dose, “S” represents staying at the same dose, and “D” represents de-escalating the dose. Decision “DU” means that the current dose level may be unacceptable because of high toxicity. A dose will be defined as having unacceptable toxicity if the posterior probability that the DLT rate is higher than 25% is more than 95%.

Enrollment in this study will occur on a cohort-by-cohort basis. Decisions on dose escalation and subsequent cohort size will be made by the sponsor in consultation with the safety monitoring committee (SMC) after completion of each cohort. At least 2 DLT-evaluable (DE) subjects will be treated per dose level until the first DLT is observed. After the first DLT is observed, a minimum of 3 DE subjects per dose level will be required before escalation to all higher doses. Upon initial evaluation of a dose level during dose escalation, 2 subjects may not receive the initial dose of study drug on the same day. Subjects who are considered not evaluable for DLT during Cycle 1 may be replaced. A minimum of 6 DE subjects will be observed at the estimated MTD before the MTD is determined. The MTD will be estimated based on data from all subjects across all evaluated doses. The MTD is defined as the highest evaluated dose that does not cause unacceptable side effects, as determined by the mTPI design.

De-escalation to a lower dose level may be performed at any time by the sponsor in consultation with the SMC.

During dose escalation, additional subjects may be enrolled at tested dose levels deemed tolerable (hereafter referred to as “backfilling”). DLTs observed at lower dose levels will be taken into account with the totality of the data; the SMC will base future escalation decisions on mTPI model recommendations at current and lower dose levels at which the backfilled DLTs were observed.

TABLE 1
Dose-Finding Spreadsheet for mTPI

| | Number of DLT-Evaluable Subjects at Current Dose | | | | | | | | | | | | | | |
|---|---|---|---|---|---|---|---|---|---|---|---|---|---|---|---|
| | | 2 | 3 | 4 | 5 | 6 | 7 | 8 | 9 | 10 | 11 | 12 | 13 | 14 | 15 |
| Number of DLTS | 0 | E | E | E | E | E | E | E | E | E | E | E | E | E | E |
| | 1 | S | S | S | S | E | E | E | E | E | E | E | E | E | E |
| | 2 | DU | D | D | S | S | S | S | S | S | S | S | S | F | E |
| | 3 | | DU | DU | DU | D | S | S | S | S | S | S | S | S | S |
| | 4 | | | DU | DU | DU | DU | DU | D | S | S | S | S | S | S |
| | 5 | | | | DU | DU | DU | DU | DU | DU | D | S | S | S | S |
| | 6 | | | | | DU | DU | DU | DU | DU | DU | DU | DU | D | S |
| | 7 | | | | | | DU | DU | DU | DU | DU | DU | DU | DU | DU |
| | 8 | | | | | | | DU | DU | DU | DU | DU | DU | DU | DU |
| | 9 | | | | | | | | DU | DU | DU | DU | DU | DU | DU |
| | 10 | | | | | | | | | DU | DU | DU | DU | DU | DU |
| | 11 | | | | | | | | | | DU | DU | DU | DU | DU |
| | 12 | | | | | | | | | | | DU | DU | DU | DU |
| | 13 | | | | | | | | | | | | DU | DU | DU |
| | 14 | | | | | | | | | | | | | DU | DU |
| | 15 | | | | | | | | | | | | | | DU |

D = De-escalate to the next lower dose;
DU = Current dose is unacceptably toxic;
E = Escalate to the next higher dose;
S = Stay at the current dose

SGN-B6A dose level and administration schedule will be defined by cohort assignment. SGN-B6A will initially be administered on Days 1, 8, and 15 of 21-day cycles at the planned doses shown in Table 2. Longer dosing intervals (e.g., Days 1 and 8 only or Day 1 only of 21-day cycles or biweekly in 28-day cycles) may be evaluated during dose escalation after consultation with the SMC. The mTPI dose escalation rules will be applied separately to each dosing schedule. The SMC may also recommend investigation of lower and/or intermediate dose levels, in which case the mTPI dose escalation rules will continue to be applied.

TABLE 2
Dose Escalation Schema

| SGN-B6A | Dose |
|---|---|
| Dose Level^{a} | (mg/kg) |
| 1 | 0.8 |
| 2 | 1.0 |
| 3 | 1.2 |
| 4 | 1.5 |
| 5 | 1.8 |
| 6 | 2.4 |

^{a}The SMC may recommend investigation of lower or intermediate dose levels based on emerging clinical data.

### Part B—Expansion Cohort

Disease-specific cohorts: At the completion of dose escalation for a particular schedule, to further characterize the safety, PK, and antitumor activity of SGN-B6A, up to 360 additional subjects may be enrolled into up to 9 disease-specific expansion cohorts. Expansion cohorts may each enroll up to approximately 40 subjects in selected tumor types eligible for enrollment in dose escalation. For each disease-specific cohort, 15 subjects will be enrolled first. For expansion cohorts, a baseline fresh tumor tissue biopsy is required, if feasible. An optional protocol-specified on-treatment or end of treatment (EOT) research biopsy may be collected for exploratory correlative studies of SGN-B6A tumor exposure and antitumor activity. A futility analysis will be performed using the Predictive Probability of Success (PPOS) approach with success being defined as having more than 0.70 posterior probability that the response rate is greater than the background response rate. Futility may be determined if the PPOS is <15% in a disease-specific cohort. When the background response rate is 0.05, if at least one objective response (OR) is observed among the first 15 subjects, up to 25 additional subjects may be enrolled; if an OR is not observed in the first 15 subjects, the SMC will assess the totality of the data and may recommend continued enrollment of up to 25 additional subjects if there is preliminary evidence of clinical benefit among the first 15 subjects. Evidence of clinical benefit may include stabilization or improvement of disease related symptoms as assessed by the investigator, or disease regression on imaging. The dose(s), schedule, and disease setting for expansion cohorts will be determined by the sponsor in consultation with the SMC and may differ between the cohorts.

Biology cohort: An additional up to 30 subjects with any of the selected tumor types who consent to protocol-specified research biopsies may be eligible to enroll in a biology cohort. Subjects in the biology cohort will be asked to provide pre- and post-treatment tumor samples to characterize the clinical mechanism of action (MOA) and correlates of sensitivity/resistance at the MTD or recommended dose.

Part C—SGN-B6A in Combination with Pembrolizumab

Part C is designed to evaluate safety and tolerability of SGN-B6A in combination with pembrolizumab. SGN-B6A will be administered starting at the recommended dose and schedule defined in Part A in combination with pembrolizumab (200 mg every 3 weeks, or 400 mg every 6 weeks). Part C will be conducted using the mTPI method with a minimum cohort size of 3. If necessary, the dose will be de-escalated to dose level-1, as determined in Part A.

### Part D—Combination Treatment Expansion Cohorts

To further characterize the safety, PK, and antitumor activity of SGN-B6A in combination with pembrolizumab, 2 expansion cohorts for NSCLC (TPS ≥50 by local testing) and HNSCC (CPS ≥1 by local testing) will enroll up to approximately 40 subjects each. The dose regimens evaluated in Part C will inform the selection of the dose(s) and schedule for the expansion cohorts, to be determined by the sponsor in consultation with the SMC. Dose(s) and schedule may differ between the cohorts.

### Duration Of Treatment

In all parts, subjects may continue treatment with SGN-B6A until disease progression, unacceptable toxicity, investigator decision, withdrawal of consent, start of a subsequent anticancer therapy, pregnancy, or study termination by the sponsor, whichever comes first. In Parts C and D, pembrolizumab may be administered until confirmed disease progression per iRECIST, unacceptable toxicity, investigator decision, withdrawal of consent, start of a subsequent anticancer therapy, pregnancy, or study termination by the sponsor, or for up to 24 months, whichever comes first.

### Dose-Limiting Toxicity

DLTs will be evaluated during dose escalation. The DLT evaluation period will be the first cycle (21 days or 28 days). Grading will be according to the National Cancer Institute Common Terminology Criteria for Adverse Events (NCI CTCAE), v5.0.

For Part A, a DLT is defined as any of the following during the DLT evaluation period if assessed by the investigator to be clinically significant and related to SGN-B6A treatment.

For Part C, a DLT is defined as any of the following that is considered related to SGN-B6A or the combination with pembrolizumab, that cannot be attributed to pembrolizumab alone.
Grade 5 toxicity
≥Grade 3 non-hematologic toxicity (not laboratory), with the following exceptions:
   Grade 3 fatigue, fever, or constipation that resolves with 72 hours, with or without intervention
   Grade 3 nausea, vomiting, or diarrhea in the absence of standard-of-care prophylaxis
   Grade 3 IRRs that resolve to ≤Grade 2 within 24 hours with or without intervention
   In the event of a Grade 3 IRR in ≥20% of subjects (ie, 2 or more in the first 10 subjects), all subsequent subjects will require premedication and/or modification of infusion approach per the recommendation of the SMC and the protocol will be amended. For subsequent subjects, any ≥Grade 3 IRR will be considered a DLT.
Grade 3 or Grade 4 non-hematologic laboratory abnormality if:
   The abnormality requires hospitalization or clinically significant medical intervention, OR
   The abnormality persists for >7 days, OR
Grade 4 anemia or thrombocytopenia
Grade 4 hematologic toxicity (other than anemia/thrombocytopenia) lasting >7 days
Grade 3 thrombocytopenia with clinically significant bleeding that requires a platelet transfusion
≥ Grade 3 febrile neutropenia
Dose delay ≥14 days due to toxicity

### Objectives

This study will evaluate the efficacy, safety, and PK of SGN-B6A in adults with the following histologically or cytologically confirmed metastatic or unresectable cancers: NSCLC, HNSCC, advanced HER2-negative breast cancer, ESCC, EAC/GEJ adenocarcinoma, HGSOC, cSCC, exocrine pancreatic adenocarcinoma, bladder cancer, cervical cancer, or gastric cancer. This study will also evaluate the efficacy, safety, and PK of SGN-B6A in combination with pembrolizumab in adults with metastatic or unresectable NSCLC or HNSCC. Specific objectives and corresponding endpoints for the study are summarized in Table 3.

TABLE 3
Objectives and corresponding endpoints

| | |
|---|---|
| Primary Objectives | Corresponding Primary Endpoints |
| To evaluate the safety and tolerability of SGN-B6A | Type, incidence, severity, seriousness, and |
| monotherapy in subjects with advanced solid tumors | relatedness of adverse events (AEs) |
| To identify the maximum tolerated dose (MTD) of | Type, incidence, and severity of laboratory |
| SGN-B6A in subjects with advanced solid tumors | abnormalities |
| To identify a recommended dose and schedule for | Incidence of dose-limiting toxicities (DLTs) |
| SGN-B6A | |
| To evaluate the safety and tolerability of SGN-B6A | |
| in combination with pembrolizumab | |
| To identify a recommended dose and schedule for | |
| SGN-B6A in combination with pembrolizumab | |
| Secondary Objectives | Corresponding Secondary Endpoints |
| To assess the antitumor activity of SGN-B6A | Confirmed objective response rate (ORR) per |
| monotherapy at the recommended dose | Response Evaluation Criteria in Solid Tumors |
| To assess the antitumor activity of SGN-B6A in | (RECIST) v1.1 by investigator |
| combination with pembrolizumab | Duration of objective response (DOR) per RECIST |
| To assess the pharmacokinetic (PK) of SGN-B6A | v1.1 by investigator |
| To assess the immunogenicity of SGN-B6A | Progression-free survival (PFS) per RECIST v1.1 |
| | by investigator |
| | Overall survival (OS) |
| | Estimates of selected PK parameters for plasma |
| | SGN-B6A antibody, antibody-conjugated MMAE, |
| | and released MMAE concentrations |
| | Incidence of anti-drug antibodies (ADAs) |
| Exploratory Objectives | Corresponding Exploratory Endpoints |
| To characterize the pharmacodynamics of SGN-B6A | Exploratory biomarkers of SGN-B6A-mediated |
| To assess PK/pharmacodynamic relationships of | pharmacodynamic effects |
| SGN-B6A | Integrin beta-6 characterization on malignant cells |
| To assess exploratory biomarkers in relation to | Correlative analysis of pharmacodynamic and PK |
| response, toxicity, PK/pharmacodynamic, and | exposure measurements |
| resistance to SGN-B6A | Correlative analysis of pharmacodynamic |
| To assess the impact of SGN-B6A on functioning, | measurements and response, toxicity, and |
| symptoms, and health-related quality of life | resistance assessments |
| (HRQoL), using established patient reported | Patient reported outcomes per validated tools: |
| outcome (PRO) instruments, in subjects with | European Organisation for Research and |
| HNSCC, ESCC, and EAC/GEJ adenocarcinoma | Treatment of Cancer (EORTC) Quality of Life |
| To assess the impact of SGN-B6A in combination | Module for Head and Neck Cancer (QLQ- |
| with pembrolizumab on functioning, symptoms, and | H&N35), Quality of Life Questionnaire for |
| HRQoL, using established PRO instruments, in | Esophageal Cancer (QLQ-OES18), Quality of Life |
| subjects with NSCLC and HNSCC | Questionnaire (QLQ-C30) and Item List 46 (IL46) |
| To assess the impact of SGN-B6A on functioning. | Descriptive outcomes of qualitative interviews |
| symptoms, and HRQoL, through one-on-one | ORR, DOR, and PFS per iRECIST by investigator |
| interviews, in subjects with NSCLC, HNSCC, | assessment |
| ESCC, and EAC/GEJ | ORR (regardless of confirmation) per RECIST |
| To assess the impact of SGN-B6A in combination | v1.1 by investigator |
| with pembrolizumab on functioning, symptoms, and | Estimates of selected PK parameters for plasma |
| HRQoL, through one-on-one interviews, in subjects | SGN-B6A antibody, antibody-conjugated MMAE, |
| with NSCLC and HNSCC | and released MMAE concentrations |
| To assess ORR, DOR, and PFS per Modified | Incidence of ADAs |
| RECIST 1.1 for Immune-based Therapeutics | Confirmed ORR per RECIST v1.1 by BICR |
| (iRECIST) for combination cohorts | DOR per RECIST v1.1 by BICR |
| To assess the PK of SGN-B6A in combination with | PFS per RECIST v1.1 by BICR |
| pembrolizumab | |
| To assess the immunogenicity of SGN-B6A in | |
| combination with pembrolizumab | |
| To assess the antitumor activity of SGN-B6A | |
| monotherapy and in combination with | |
| pembrolizumab using blinded independent central | |
| review (BICR) | |

### STUDY ENDPOINT DEFINITIONS

Objective Response Rate—The ORR (regardless of confirmation) is defined as the proportion of subjects achieving a PR or CR per RECIST v1.1. Confirmed ORR is defined as the proportion of subjects with CR or PR which is subsequently confirmed as assessed according to RECIST v1.1. Subjects whose disease response cannot be evaluated per the response criteria will be scored as “not evaluable” for calculating the ORR. Subjects who do not have a postbaseline response assessment or have a response that is “not evaluable” will be counted as nonresponders in the calculation of ORR.

Progression Free Survival-PFS is defined as the time from the start of any study treatment to first documentation of PD (based on radiographic assessments per RECIST v1.1) or to death due to any cause, whichever comes first. Subjects who are alive and not progressed at the time of analysis will be censored at the date of last tumor assessment. Subjects lacking an evaluation of tumor response after their first dose will be censored at 1 day.

Overall Survival—OS is defined as the time from the start of any study treatment to the date of death due to any cause. Specifically,

OS=date of death−date of first dose of any study treatment+1.

OS for subjects who are alive at their date of last contact, including those lost to follow-up, will be censored at the date of last contact. If the last recorded date where a subject is known to be alive is the date of first dose of any study treatment, survival time will be censored on the date of first dose of any study treatment (i.e., OS duration of 1 day).

Duration of Response-DOR is defined as the time from the first documentation of objective response (CR or PR that is subsequently confirmed) to the first documentation of PD or to death due to any cause, whichever comes first. DOR data will be censored on the date of the last adequate disease assessment documenting absence of PD for subjects who do not have tumor progression and are still on study at the time of an analysis, are given antitumor treatment other than the study treatment, or are removed from study prior to documentation of tumor progression. DOR will only be calculated for the subjects achieving a confirmed response.

### Study Population

Subjects must meet all of the enrollment criteria to be eligible for this study. Eligibility criteria may not be waived by the investigator and are subject to review in the event of a good clinical practice audit and/or health regulatory authority inspection.

### Inclusion Criteria

1. Disease indication
a. For Part A:
   Subjects must have histologically or cytologically confirmed metastatic or unresectable solid malignancy within one of the tumor types listed below. Subjects must have disease that is relapsed or refractory or be intolerant to standard-of-care therapies, and in the judgement of the investigator, should have no appropriate standard-of-care therapeutic option. If a standard-of-care therapy is available that has not been administered, the reason that the therapy is not appropriate must be documented.
      NSCLC
      HNSCC
      advanced HER2-negative breast cancer
      ESCC
      EAC/GEJ
      HGSOC
      cSCC
      exocrine pancreatic adenocarcinoma
      bladder cancer
      cervical cancer
      gastric cancer
b. For Part B:
   Subjects may be entered into up to 9 disease specific expansion cohorts. Subjects must have histologically or cytologically confirmed disease that is relapsed or refractory, or be intolerant to standard-of-care therapies as specified below, unless contraindicated:
      NSCLC:
         Subjects must have locally advanced or metastatic non-small cell lung cancer
         Prior therapy: subjects must have received platinum-based therapy and a PD-1/PD-L1 inhibitor, if applicable and available per local standard of care. These agents may have been administered either as single agents or in combination.
         Subjects with documented actionable genomic alterations should have received treatment with an approved tyrosine kinase inhibitor (TKI; eg, ALK, ROS-1 gene rearrangement, or EGFR mutation, or other applicable targeted therapy) for the respective genomic alteration per local guidelines. These subjects should have received no more than 3 lines of systemic therapy in the locally advanced or metastatic setting. Maintenance therapy should not be counted as a separate line of therapy.
      HNSCC:
         Subjects must have locally advanced or metastatic head and neck squamous cell cancer.
         Prior therapy: subjects must have received platinum-based therapy and a PD-1/PD-L1 inhibitor, if eligible by biomarker status and local standard of care. These agents may have been administered either as single agents or in combination
      Advanced HER2-negative breast cancer:
         Subjects must have locally advanced or metastatic HER2-negative breast cancer (per American Society of Clinical Oncology/College of American Pathologists [ASCO/CAP] 2018 guidelines).
         Subjects must have received 1 or more prior lines of therapy for locally advanced or metastatic disease. Prior therapies must include a taxane administered cither as single agent or in combination.
         Hormone-receptor-positive subjects additionally must have received CDK4/6-inhibitor therapy unless contraindicated, and at least 1 prior hormonally-directed therapy.
         If eligible by biomarker status and consistent with standard-of-care, must have received a poly-ADP ribose polymerase (PARP) inhibitor, PD-1/PD-L1 inhibitor, and/or phosphoinositide 3-kinase (PI3K) inhibitor.
      ESCC:
         Subjects must have locally advanced or metastatic esophageal squamous cell carcinoma.
         Subjects must have received prior platinum-based chemotherapy.
         If eligible by biomarker status and consistent with standard-of-care, must have received a prior PD-1/PD-L1 inhibitor.
      EAC/GEJ adenocarcinoma:
         Subjects must have locally advanced or metastatic EAC or GEJ adenocarcinoma.
         Subjects must have received prior platinum-based chemotherapy.
         If eligible by biomarker status and consistent with standard-of-care, must have received a prior PD-1/PD-L1 inhibitor, or HER-2 directed therapy.
      HGSOC:
         Subjects must have high grade serous epithelial ovarian cancer, primary peritoneal cancer, or fallopian tube cancer.
         Subjects must have platinum-resistant disease, which is defined as having progressed or relapsed within 6 months after previous platinum-containing chemotherapy. If eligible, must have received a bevacizumab-containing regimen. If eligible by biomarker status and consistent with standard-of-care, must have received a PARP inhibitor.
      cSCC:
         Subjects must have locally advanced or metastatic cutaneous squamous cell carcinoma.
         Subjects must have received aPD-1/PD-L1 inhibitor.
c. For Part C:
   Subjects must have histologically or cytologically confirmed metastatic or unresectable solid malignancy within one of the tumor types listed below.
      NSCLC
      HNSCC
      ESCC
      cSCC
   Subjects must be eligible for pembrolizumab monotherapy per local standard of care.
d. For Part D:
   Subjects may be entered into up to 2 disease specific expansion cohorts. Subjects must have histologically or cytologically confirmed disease, and have not received frontline systemic therapy for locally advanced or metastatic disease (prior definitively intended or adjuvant therapy is allowed with the exception of PD-[L]1).
   Subjects with NSCLC must have TPS ≥50 by local testing to be eligible for the cohort testing SGN-B6A in combination with pembrolizumab.
Subjects with HNSCC must have CPS ≥1 by local testing to be enrolled to the cohort testing SGN-B6A in combination with pembrolizumab.
2. Subjects enrolled in the following study parts should have a tumor site accessible for biopsy and agree to biopsy as follows:
   Disease-specific expansion cohorts (Part B and Part D): A baseline fresh tumor biopsy is required. An archival biopsy collected within 90 days may be used. This biopsy can be waived if medically infeasible after discussion with medical monitor (e.g., lesions are not accessible with a minimally invasive procedure that does not represent significant risk)
   Biology expansion cohort: pretreatment biopsy and on-treatment (Cycle 1) biopsy Additional optional biopsies are requested, if feasible for all subjects in the disease-specific expansion and biology cohorts.
3. Age 18 years or older.
4. An Eastern Cooperative Oncology Group (ECOG) Performance Status score of 0 or 1.
5. Measurable disease per the RECIST v1.1 at baseline.
6. The following baseline laboratory data:
   absolute neutrophil count (ANC) ≥1500/μL
   hemoglobin (Hgb) ≥9 g/dL
   platelet count ≥100,000/μL
   serum bilirubin≤1.5× upper limit of normal (ULN) or ≤3×ULN for subjects with Gilbert's disease
   estimated glomerular filtration rate (GFR)≥45 mL/min/1.73 m² using the Modification of Diet in Renal Disease (MDRD) study equation as applicable
   alanine aminotransferase (ALT) and aspartate aminotransferase (AST)≤3×ULN (≤5×ULN if there is evidence of hepatic involvement by malignant disease)
7. Subjects of childbearing potential under the following conditions:
   a. Must have a negative serum or urine pregnancy test (minimum sensitivity 25 mIU/mL or equivalent units of beta human chorionic gonadotropin [β-hCG]) result within 7 days prior to the first dose of SGN-B6A. Subjects with false positive results and documented verification that the subject is not pregnant are eligible for participation.
   b. Must agree not to try to become pregnant during the study and for at least 2 months after the final dose of study drug administration.
   c. Must agree not to breastfeed or donate ova, starting at time of informed consent and continuing through 2 months after the final dose of study drug administration.
   d. If sexually active in a way that could lead to pregnancy, must consistently use at least 2 acceptable methods of birth control (contraception) at least one of which must be highly effective starting at time of informed consent and continuing throughout the study and for at least 2 months after the final dose of study drug administration.
8. Subjects who can get someone pregnant, under the following conditions:
   a. Must agree not to donate sperm starting at time of informed consent and continuing throughout the study period and for at least 4 months after the final study drug administration.
   b. If sexually active with a person of childbearing potential in a way that could lead to pregnancy, must consistently use at least 2 acceptable methods of birth control (contraception), at least 1 of which must be highly effective starting at time of informed consent and continuing throughout the study and for at least 4 months after the final dose of study drug administration.
   c. If sexually active with a person who is pregnant or breastfeeding, must consistently use a condom starting at time of informed consent and continuing throughout the study and for at least 4 months after the final dose of study drug administration.
9. The subject must provide written informed consent.

### Exclusion Criteria

1. History of another malignancy within 3 years before the first dose of study drug, or any evidence of residual disease from a previously diagnosed malignancy. Exceptions are malignancies with a negligible risk of metastasis or death (e.g., 5-year OS ≥90%), such as adequately treated carcinoma in situ of the cervix, non-melanoma skin carcinoma, localized prostate cancer, ductal carcinoma in situ, or Stage I uterine cancer.
2. Known active central nervous system metastases. Subjects with previously treated brain metastases may participate provided they are clinically stable for at least 4 weeks prior to study entry after brain metastasis treatment, they have no new or enlarging brain metastases, and are off of corticosteroids prescribed for symptoms associated with brain metastases for at least 7 days prior to the first dose of study drug.
3. Carcinomatous meningitis.
4. Previous receipt of an MMAE-containing agent or an agent targeting integrin beta-6.
5. Pre-existing neuropathy ≥Grade 2 per NCI CTCAE v5.0.
6. Any uncontrolled ≥Grade 3 (per the NCI CTCAE, v5.0) viral, bacterial, or fungal infection within 2 weeks prior to the first dose of SGN-B6A. Routine antimicrobial prophylaxis is permitted.
7. Uncontrolled diabetes mellitus, defined as hemoglobin A1C (Hgb A1c) ≥8.0% or Hgb A1c between 7 and <8.0% with associated diabetes symptoms (polyuria or polydipsia) that are not otherwise explained.
8. Positive for hepatitis B by surface antigen expression. Active hepatitis C infection (positive by polymerase chain reaction [PCR] or on antiviral therapy for hepatitis C within the last 6 months). Subjects who have been treated for hepatitis C infection are permitted if they have documented sustained virologic response of 12 weeks.
9. Known to be positive for human immunodeficiency virus (HIV).
10. Documented history of a cerebral vascular event (stroke or transient ischemic attack), unstable angina, myocardial infarction, congestive heart failure, or cardiac symptoms consistent with New York Heart Association Class III-IV within 6 months prior to their first dose of SGN-B6A.
11. Congestive heart failure, Class III or IV, by the New York Heart Association criteria.
12. Pulmonary disease ≥Grade 3 unrelated to underlying malignancy.
13. During dose escalation only, use of strong cytochrome P450 3A (CYP3A) inhibitors within 14 days of study drug dosing.
14. Chemotherapy, immunotherapy, biologics, and/or other approved or investigational antitumor treatment that is not completed 4 weeks prior to first dose of study drug, or within 2 weeks prior to the first dose of study drug if the underlying disease has progressed on treatment.
15. Focal radiotherapy or major surgery that is not completed 2 weeks prior to the first dose of SGN-B6A.
16. Subjects who are breastfeeding, pregnant, or planning to become pregnant from time of informed consent until 2 months after final dose of study drug administration.
17. Known hypersensitivity to any excipient contained in the drug formulation of SGN-B6A.
18. Estimated life expectancy <12 weeks
19. Other serious underlying medical condition that, in the opinion of the investigator, would impair the subject's ability to receive or tolerate the planned treatment and follow-up.
20. Subjects who received live vaccines within 30 days of first study drug dose.

### Part C Only:

21. Has received prior therapy with a PD-1 inhibitor, anti-PD-L1, or anti PD-L2 agent or with an agent directed to another stimulatory or co-inhibitory T-cell receptor (e.g., CTLA-4, OX 40, CD137) and was discontinued from that treatment due to a Grade 3 or higher immune-mediated adverse event (IMAE).

### Part D Only:

22. Has NSCLC with an actionable genomic alteration (e.g., ALK, ROS-1 gene rearrangement, or EGFR mutation).

### Parts C and D Only:

23. Has a diagnosis of immunodeficiency or is receiving chronic systemic steroid therapy (in dosing exceeding 10 mg daily of prednisone equivalent) or any other form of immunosuppressive therapy within 7 days prior the first dose of study drug.
24. Has an active autoimmune disease that has required systemic treatment in past 2 years (i.e., with use of disease modifying agents, corticosteroids or immunosuppressive drugs). Replacement therapy (e.g., thyroxine, insulin, or physiologic corticosteroid replacement therapy for adrenal or pituitary insufficiency) is not considered a form of systemic treatment and is allowed.
25. Has a history of (non-infectious) pneumonitis that required steroids or has current pneumonitis.
26. Has a history of interstitial lung disease.
27. Has received radiation therapy to the lung that is >30 Gy within 6 months of the first dose of trial treatment.
28. Has had an allogeneic tissue/solid organ transplant.

### Treatments Administered

All subjects will receive SGN-B6A, the investigational agent under study in this protocol. SGN-B6A is an ADC composed of an anti-integrin beta-6 antibody (h2A2) conjugated to the tubulin-disrupting antimitotic agent MMAE. Subjects in Part C and Part D will also receive pembrolizumab.SGN-B6A is a sterile, preservative-free white to off-white lyophilized cake or powder for reconstitution for IV administration. SGN-B6A is supplied in single-dose glass vials. When reconstituted with Water for Injection (WFI), United States Pharmacopoeia (USP), or equivalent, the reconstituted drug product is a clear to slightly opalescent, colorless to slightly yellow solution with no visible particulate matter.

### Dose and Administration

SGN-B6A dose level and administration schedule will be defined by cohort assignment. SGN-B6A will be administered by IV infusion. Dosing frequency (continuous, intermittent, Q3W or biweekly) is outlined below. Subjects in the dose escalation phase will be required to undergo a 4-hour observation period on Cycle 1 Day 1 following completion of the infusion. It is recommended that subjects remain in close proximity to a medical facility for 24 hours after Cycle 1 Day 1 administration of SGN-B6A to enable prompt evaluation in case of a delayed IRR. If the first infusion is tolerated well and no IRR occurs, the observation period for subsequent infusions may be reduced as per institutional standard.

SGN-B6A will initially be administered on Days 1, 8, and 15 of a 21-day cycle. A decreased frequency of dosing, such as dosing on Days 1 and 8 only, or Day 1 only, of every 21-day cycle or biweekly of every 28-day cycle, may be implemented if recommended by the SMC. In no case should doses of SGN-B6A be given fewer than 5 days apart.

### Detailed Dosing Regimen

21-DAY CYCLE
Continuous The study drug will be administered to study subjects on a weekly basis (e.g., Cycle 1 Day 1, Cycle 1 Day 8, Cycle 1 Day 15, then Cycle 2 Day 1, Cycle 2 Day 8, Cycle 2 Day 15).
Intermittent: The study drug will be administered to study subjects on Days 1 and 8 of each cycle with no administration on Day 15 (e.g., Cycle 1 Day 1, Cycle 1 Day 8, then Cycle 2 Day 1, Cycle 2 Day 8).
Q3w: The study drug will be administered to study subjects once every 3 weeks (e.g., Cycle 1 Day 1, then Cycle 2 Day 1).
28-DAY CYCLE
Biweekly: The study drug will be administered to study subjects on a biweekly basis (e.g., Day 1 and Day 15 of each cycle).

SGN-B6A requires dilution prior to administration.

Weight-based dosing is based on the subject's body weight at baseline, or per institutional standards. The SMC may recommend implementing Adjusted Ideal Body Weight (AIBW) dosing if indicated. Doses must be adjusted for subjects who experience a ≥10% change in weight from baseline. Other dose adjustments for changes in body weight <10% from baseline are permitted per institutional standard. Rounding to the nearest whole number of milligrams is permissible within 5% of the nominal dose.

Infusion duration will vary depending on the method of infusion administration and the dose. The rate of infusion should be per institutional standard but should not exceed 250 mL/hour. In dose escalation Part A, SGN-B6A should be infused over a approximately 30 minutes. Upon review of aggregate subject data, the SMC may recommend or require a longer or shorter infusion duration.

If an individual subject does not tolerate the infusion, the infusion duration for that subject may be increased; the infusion duration in subsequent infusions may also be increased per investigator discretion with medical monitor consultation. Conversely, if a subject tolerates consecutive infusions without IRR >Grade 1, the infusion duration may be shortened (i.e., administered at a faster rate) at the discretion of the investigator with medical monitor consultation, the implementation of which may be dose-cohort specific.

The infusion site should be monitored closely for redness, swelling, pain, and infection during and at any time after administration. Subjects should be advised to report redness or discomfort promptly at the time of administration or after infusion. Institutional guidelines will be followed for the administration of chemotherapy agents and precautions taken to prevent extravasation per institutional standards.

As clinical experience with SGN-B6A progresses, alternative approaches to SGN-B6A administration and dosing may be evaluated, including but not limited to administration at a fixed infusion rate, weight-capped dose administration, AIBW dose administration, or flat dose administration.

If a fixed infusion rate is implemented upon SMC recommendation, the dose is administered at a fixed rate rather than over a fixed time.

For example, for a fixed infusion rate of 50 mg/hr, a dose of 100 mg would be infused over 2 hours. As clinical experience with administration at a fixed infusion rate evolves, the rate may be increased, or decreased, based on accumulating safety data and/or recommendations of the SMC.

As PK, pharmacodynamics, and clinical activity data evolve, weight-capped IV dose administration may be implemented upon SMC recommendation. In contrast to weight-based dosing, in which the total dose is calculated with no upper limit on subject weight, weight-capped dosing limits the weight to be used for the total dose calculation. The upper limit to be used in dose calculation is to be defined based on emerging data.

For example, if the weight cap is 100 kg, a subject with body weight ≥100 kg will use a weight of 100 kg to calculate the total dose to be administered. In a subject with body weight <100 kg, the total dose to be administered is calculated using the subject's actual weight.

AIBW provides adjustment to ideal body weight (IBW) if the subject's actual total body weight (TBW) is higher or lower than their IBW. Since AIBW is calculated using subjects' gender, height, and TBW, the percent adjustment to total dose will depend on the target body mass index (BMI) groups.

### Dose Modifications

For SGN-B6A, dose reductions or dosing interval lengthening for toxicity, including DLT, may be approved on a per-subject basis, by the investigator in consultation with the medical monitor. For subjects treated at the lowest dose level, the dose may be lowered to 25% of the most recently administered dose, the dosing frequency may be decreased (e.g., if initial dosing is Days 1, 8, and 15, dosing may be altered to Days 1 and 8, or Day 1 only in a 21-day cycle), or the subject may be discontinued from treatment.

Subjects who experience DLT in Cycle 1 should not receive further treatment with SGN-B6A unless toxicity is adequately managed, the investigator considers resumption of SGN-B6A to be appropriate, and there is consultation with the medical monitor. The type and severity of the AE observed will be taken into consideration to inform the decision. If a subject continues treatment after DLT and the same dose-limiting toxicity recurs, treatment must be permanently discontinued. Subjects who experience AEs that meet the criteria for permanent discontinuation of SGN-B6A may not resume SGN-B6A, including at a lower or modified dose or decreased dosing frequency.

If a subject has a clinically significant, unresolved treatment-emergent adverse event (TEAE) on the next scheduled dosing day of Cycle 1 or beyond, the next dose may be delayed for up to 7 days. Delays due to other reasons or lasting >7 days must be discussed with the medical monitor. Subjects requiring a dose delay >7 days due to an unresolved TEAE may be dosed in subsequent cycles at a reduced dose or the dosing frequency may be decreased with medical monitor consultation.

During the DLT period, growth factor and transfusion support is discouraged unless medically indicated; subjects who receive growth factor (eg, granulocyte colony stimulating factor [G-CSF] or granulocyte macrophage colony stimulating factor [GM-CSF]) or transfusion support during this period for reasons other than DLT may not be evaluable for DLT. Consideration should be given to growth factor support for prophylaxis or treatment of cytopenias in subsequent cycles. Discontinuation or dose reduction to 1 dose level below the current dose (see Table 2) may be considered for subjects with recurrent Grade 4 neutropenia despite the use of growth factors.

A comprehensive metabolic panel (CMP) and complete blood count (CBC) should be collected at least weekly during dose delays. If AEs other than neutropenia recur despite 2 dose reductions, an additional dose reduction may be granted following discussion with the medical monitor.

Table 4 describes recommended dose modifications for study treatment-associated toxicity. Medical monitor consultation is required for continued study treatment after occurrence of a ≥Grade 3 AE potentially associated with SGN-B6A treatment.

TABLE 4
Recommended dose modifications for potential SGN-B6A-associated toxicity

| Toxicity | Grade 1 | Grade 2 | Grade 3^{a} | Grade 4^{a} |
|---|---|---|---|---|
| Neutropenia | Continue at same | Continue at same | Withhold until | Withhold until resolution to |
| | dose. | dose. | resolution to | ≤Grade 2. |
| | | | ≤Grade 2. | Strongly consider growth factor |
| | | | Consider growth | support for treatment of Grade 4 |
| | | | factor support for | neutropenia. |
| | | | treatment of | Resume study treatment at same |
| | | | Grade 3 | dose. |
| | | | neutropenia. | Strongly consider prophylactic |
| | | | Resume study | growth factor support for |
| | | | treatment at | subsequent cycles. |
| | | | the same dose. | If recurrence, consider dose |
| | | | Consider | reduction, upon consultation with |
| | | | prophylactic | the medical monitor, or |
| | | | growth factor | discontinue treatment, at the |
| | | | support for | investigator's discretion^{b}. |
| | | | subsequent | |
| | | | cycles. | |
| | | | If recurrence, | |
| | | | consider dose | |
| | | | reduction, upon | |
| | | | consultation with | |
| | | | the medical | |
| | | | monitor, or | |
| | | | discontinue | |
| | | | treatment, at the | |
| | | | investigator's | |
| | | | discretion. | |
| Febrile | NA | NA | Withhold until complete resolution. | |
| neutropenia | | | Administer growth factor support. | |
| | | | Resume study treatment at same dose, with prophylactic | |
| | | | growth factor support. Per investigator clinical judgement | |
| | | | a dose reduction may be applied, upon consultation with | |
| | | | medical monitor. | |
| | | | If recurrence, consider dose reduction, upon consultation | |
| | | | with the medical monitor, or discontinue treatment, at the | |
| | | | investigator's discretion^{b}. | |
| Other hematologic^{c} | Continue at same | Continue at same | Withhold until | Withhold until resolution to |
| toxicity | dose. | dose. | resolution to | ≤Grade 2 or baseline. |
| | | | ≤Grade 2 or | Resume study treatment at same |
| | | | baseline. | dose. |
| | | | Resume study | |
| | | | treatment at same | |
| | | | dose | |
| Peripheral | Continue at same | Withhold until | Withhold until | Permanently discontinue treatment |
| neuropathy | dose level | toxicity resolves to | toxicity resolves to | |
| | | ≤Grade 1; then | ≤Grade 1, then | |
| | | resume treatment at | resume treatment at | |
| | | the next lower | the next lower | |
| | | dose level^{b} | dose level^{b} | |
| Oral/pharyngeal | Continue at the | Withhold until | Withhold until | Permanently discontinue treatment |
| mucositis | same dose level. | toxicity resolves to | toxicity resolves to | |
| | Implement steroid- | ≤Grade 1; then | ≤Grade 1, systemic | |
| | containing mouthwash | resume treatment at | steroids if indicated | |
| | regimen for | the next lower dose | per investigator | |
| | subsequent dosing | level. Implement | clinical judgement. | |
| | | steroid-containing | Resume treatment at | |
| | | mouthwash regimen | the next lower | |
| | | for subsequent dosing | dose level^{b} | |
| | | | Implement steroid- | |
| | | | containing | |
| | | | mouthwash | |
| | | | regimen for | |
| | | | subsequent dosing | |
| Hyperglycemia | Recommend to withhold dose for blood glucose >250 mg/dL or >13.9 mmol/L. Subjects with | | | |
| | diabetes or other confounding factors who have a higher blood glucose level may be allowed to | | | |
| | receive the dose per investigator clinical judgement, in consultation with the medical monitor. | | | |
| | Resume treatment once elevated blood glucose has improved to ≤250 mg/dL or ≤13.9 mmol/L | | | |
| | and subject is clinically and metabolically stable. | | | |
| Other | Continue at same | Continue at same | Withhold dose until | Withhold dose until toxicity is |
| non-hematologic | dose level | dose level | toxicity is ≤Grade 2 | ≤Grade 2 or has returned to |
| toxicities | | | or has returned to | baseline, then consider dose |
| | | | baseline, then | reduction to the next lower |
| | | | resume treatment at | dose level^{b} and resume |
| | | | same dose level | treatment with medical |
| | | | | monitor consultation or discontinue |
| | | | | treatment at the discretion of the |
| | | | | investigator. |
| Anaphylaxis^{d} | N/A | N/A | Immediately | Immediately and permanently |
| | | | and | discontinue study treatment. |
| | | | permanently | |
| | | | discontinue | |
| | | | study | |
| | | | treatment. | |

NA = not applicable;
IRR = infusion related reaction;
AE = adverse event.
Note:
For subjects at the lowest dose level, dose reduce by 25% (e.g. 0.6 mg/kg for subjects treated at 0.8 mg/kg prior to dose modification). Discuss treatment delays of >7 days with the medical monitor.
^{a}Medical monitor consultation is required for continued study treatment after occurrence of a ≥Grade 3 AE potentially associated with SGN-B6A treatment.
^{b}If toxicity recurs at Grade 3 or higher despite dose reduction, the dosing frequency may be decreased with medical monitor consultation.
^{c}For all grades of febrile neutropenia, follow dose modification guidance for Grade 4 hematologic toxicity.
^{d}If a subject experiences a Grade 4 IRR, allergic reaction, or anaphylaxis, the study drug must be permanently discontinued

### Pembrolizumab

Pembrolizumab is a humanized monoclonal antibody that blocks the interaction between PD-1 and its ligands, PD-L1 and PD-L2. Pembrolizumab is an IgG4 kappa Ig with an approximate molecular weight of 149 kDa.

Pembrolizumab will be supplied as a 100 mg/4 mL (25 mg/mL) solution in a single-use vial. Pembrolizumab for injection is a sterile, preservative-free, clear to slightly opalescent, colorless to slightly yellow solution that requires dilution for IV infusion. Each vial contains 100 mg of pembrolizumab in 4 mL of solution. Each 1 mL of solution contains 25 mg of pembrolizumab and is formulated in L-histidine, polysorbate, sucrose, and WFI USP.

Study treatment of pembrolizumab will be administered in Part C and D cohorts on Day 1 of each 21-day cycle or every 6 weeks at a dose of 200 mg or 400 mg, respectively, using a 30-minute IV infusion, prior to the infusion of SGN-B6A. The administration of pembrolizumab must be completed at least 30 minutes prior to the initiation of administration of SGN-B6A. Sites should make every effort to target infusion timing to be as close to 30 minutes as possible. However, given the variability of infusion pumps from site to site, a window between-5 minutes and +10 minutes is permitted (ie, infusion time is 30 minutes-5 min/+10 min).

Unless otherwise specified, administration of study treatment drug should be performed as per Product Information or institutional guidelines.

### Dose Modifications

Please refer to the pembrolizumab EU SmPC for dose modification and toxicity management guidelines for pembrolizumab.

For Parts C and D only: AEs may be attributable to 1 trial treatment alone or the combination of trial treatments. The final decision regarding causality is at the discretion of the investigator. If the event is clearly related to one of the agents, follow the instructions specific for that agent. If the event is related to more than 1 agent, follow the instructions for all agents to which it is related. There may be circumstances when a subject may not tolerate combination therapy but may benefit from treatment with 1 agent alone. These cases must be discussed and approved by the sponsor before the subject may continue treatment. In a situation where attribution to an individual study treatment may be difficult, the management actions to withhold or discontinue study treatment should apply to both SGN-B6A and pembrolizumab.

### Response/Efficacy Assessments—Clinical Response Per RECIST

Response will be assessed by radiographic tumor evaluation on Days 15-21 of 21-day cycles (Cycles 2, 4, 6 and every 3rd cycle thereafter) or every 6 weeks +/−7 days from Cycle 1 Day 1 (C1D1) in a 28-day cycle through Week 24 (4 response assessments every 6 weeks), then every 8 weeks thereafter. Subjects who discontinue treatment prior to disease progression or starting a new anticancer therapy will receive physical exams and continue to be assessed for response according to this schedule for up to 3 years after EOT until PD is documented or a new therapy is initiated. Tumor evaluation will be performed by CT and/or MRI scan of the chest, abdomen, and pelvis. Imaging of the neck must also be obtained if there is documented or suspected involvement in this region. Scans must be of diagnostic quality, and IV contrast must be used unless medically contraindicated. For each assessed lesion, the same modality should be used throughout the duration of the study.

The determination of antitumor activity will be based on assessments as defined by RECIST v1.1 for all cohorts. Treatment decisions by the investigator will be based on RECIST v1.1 for Parts A and B and iRECIST for Parts C and D. Treatment beyond disease progression per RECIST v1.1 may be considered for subjects in Parts C and D who are deriving clinical benefit per investigator and who do not meet stopping criteria. Subjects treated beyond disease progression per RECIST v1.1 may continue until confirmed disease progression per iRECIST as assessed by the investigator. Confirmatory scans must be performed 4 to 9 weeks after initial date of disease progression.

### Pharmacokinetic and Immunogenicity Assessments

Blood samples for PK and ADA assessment will be collected at time points for the applicable collection schedules.

SGN-B6A ac-MMAE, total antibody (TAb), and MMAE concentrations in plasma will be determined using validated assays. The assays may include ELISAs or liquid chromatography with tandem mass spectrometry (LC-MS/MS), as well as other assays if further characterization is required. Analysis of SGN-B6A ADC concentrations may be run in a subset of samples in an exploratory way using non-validated methods. Remaining PK samples will be archived for possible analysis of SGN-B6A-related species. PK and ADA samples will be collected for pembrolizumab in Part D cohorts and held; may be analyzed as an exploratory analysis if deemed necessary.

A qualified electrochemiluminescence assay will be used to assess levels of ADA against SGN-B6A in serum.

### Pharmacodynamic and Biomarker Assessments

Biomarker assessments will be performed in peripheral blood and tumor tissue as outlined in this section. Biomarker assessments will not be used for subject selection. Exploratory, predictive, and prognostic biomarkers associated with response, resistance, or safety observations will be monitored before and during treatment with SGN-B6A. Pharmacodynamic assessments will include disease burden monitoring, changes to target expression, and soluble B6 integrin in the plasma. Correlative studies will be conducted to gain a better understanding of target-response relationship, predictive/prognostic biomarkers, MOA, resistance mechanisms, and pharmacodynamics. On-treatment and EOT biopsies, obtained in the biology cohort and potentially in disease-specific expansions cohorts (optional biopsy), will be used to investigate the mechanism of action (MOA) and resistance mechanisms of SGN-B6A.

Tumor sample requirements vary depending on which cohort a subject is enrolled as described below. If a biopsy is not feasible or appropriate, the biopsy may be waived after discussion with the medical monitor.

If a tumor sample is obtained as part of standard of care during the study, with the subject's consent, a part of that sample should be submitted to the sponsor for biomarker testing. Biopsies should be collected by appropriately trained clinical site personnel (e.g., an interventional radiologist for internal tumor biopsies; trained personnel such as a dermatologist for cutaneous tumor biopsies). It is strongly recommended that a pathologist be present during biopsies when feasible to ensure sufficient tumor content of the biopsy location, and to confirm that biopsy acquisition and processing techniques are optimal.

The primary effects of SGN-B6A on tumor cells may lead to changes in the activation state of local, tumor-associated, and peripheral immune cells. To characterize the malignancy and immune response, biomarker assessments in peripheral blood may include, but are not limited to, measurement of baseline and drug-induced changes in circulating blood cell populations, immunoassays, gene expression, cytogenetics, genetic polymorphisms, somatic mutations associated with cancer, and circulating immune function and disease markers. SGN-B6A interactions with peripheral blood cells and tissues may also be monitored. Assays may include, but are not limited to, next generation sequencing of whole blood, proteomic methodologies, immunoassays as a marker of tumor response or therapy resistance, and markers of immune function, including abundance of immune cell subsets and cytokines. Methods of analysis may include, but are not limited to, IHC, next generation sequencing of RNA and DNA, and immunoassays such as flow cytometry and ELISA. These may provide insight into treatment-related changes associated with SGN-B6A.

To understand the relationship between the biological characteristics of tumors before treatment and subject outcomes, tissue from pre-treatment (archival or freshly obtained specimens), on-treatment and EOT tumor biopsies will be examined. If tissue is available from a standard clinical care biopsy collected after enrollment, it may also be examined. Biopsies will be assessed for specific pharmacodynamic, predictive, and prognostic biomarkers in the tumor. To characterize the penetration of SGN-B6A into the tumor, malignancy and response to study treatment, biomarker assessments in tumor biospecimens may include, but are not limited to, measurements of SGN-B6A and its potential metabolites, such as MMAE, as well as characterization of the tumor microenvironment, drug target(s), tumor subtyping, profiling of somatic mutations and/or gene expression. Assays may include, but are not limited to, LC-MS/MS, IHC, and next generation sequencing of RNA and DNA.

### Demographics and Characteristics at Baseline (Dose Escalation)

Table 5 shows the demographics and characteristics at baseline of the subjects that have been enrolled in the study.

TABLE 5
Subject demographics and characteristics

| | Q1W | 2Q3W | Q3W | 2Q4W | All Schedules |
|---|---|---|---|---|---|
| | 0.8, 1.0, 1.2 | 1.2, 1.25 | 1.5, 1.8 | 1.5, 1.8, 2.0 | All Dose |
| | mg/kg | mg/kg | mg/kg | mg/kg | Levels |
| | (N = 30) | (N = 18) | (N = 8) | (N = 23) | (N = 79) |
| Age, median years (range) | 60.0 (36-84) | 61.0 (48-78) | 56.0 (48-73) | 69.0 (46-78) | 62.0 (36-84) |
| Sex, n (%) | | | | | |
| Male | 11 (36.7) | 9 (50) | 2 (25.0) | 13 (56.5) | 35 (44.3) |
| Female | 19 (63.3) | 9 (50) | 6 (75.0) | 10 (43.5) | 44 (55.7) |
| ECOG Performance Status, n (%) | | | | | |
| 0 | 11 (36.7) | 9 (50) | 2 (25.0) | 5 (21.7) | 27 (34.2) |
| 1 | 19 (63.3) | 9 (50) | 6 (75.0) | 18 (78.3) | 52 (65.8) |
| Number of prior systemic metastatic | 2.0 (1-19) | 3.0 (1-7) | 5.0 (2-8) | 4.0 (1-7) | 3.0 (1-19) |
| therapies, median (range) | | | | | |
| Disease Diagnosis, n (%) | | | | | |
| Head and Neck Squamous Cell Cancer | 3 (10) | 7 (39) | 1 (13) | 4 (17) | 15 (19) |
| Breast Cancer | 10 (33) | 4 (22) | 1 (13) | 0 | 15 (19) |
| Non-Small Cell Lung Cancer | 3 (10) | 4 (22) | 3 (38) | 17 (74) | 27 (34) |
| Esophageal Cancer | 4 (13) | 3 (17) | 3 (38) | 2 (9) | 12 (15) |
| Cutaneous Squamous Cell Cancer | 3 (10) | 0 | 0 | 0 | 3 (4) |
| Ovarian Cancer | 3 (10) | 0 | 0 | 0 | 3 (4) |
| Exocrine Pancreatic Adenocarcinoma | 2 (7) | 0 | 0 | 0 | 2 (3) |
| Bladder Cancer | 1 (3) | 0 | 0 | 0 | 1 (1) |
| Cervical Cancer | 1 (3) | 0 | 0 | 0 | 1 (1) |

### Results

Table 6 shows the objective response rate of the subjects in the dose escalation study that have been evaluated. Subjects with all tumor types are included.

TABLE 6
Objective response rate

| | Q1W | 2Q3W | Q3W | 2Q4W |
|---|---|---|---|---|
| | 0.8, 1.0, | 1.2, | 1.5, | 1.5, |
| | 1.2 mg/kg | 1.25 mg/kg | 1.8 mg/kg | 1.8, 2.0 mg/kg |
| | N = 30 | N = 18 | N = 8 | N = 23 |
| cORR^{a}, n (%) | 4 (13.3) | 4 (22.2) | 2 (25.0) | 7 (30.4) |
| 95% CI^{b} | (3.8, 30.7) | (6.4, 47.6) | (3.2, 65.1) | (13.2, 52.9) |
| Best Overall Response^{c} | | | | |
| (Overall, n[%]) | | | | |
| cCR^{d} | 0 | 0 | 0 | 1 (4.3) |
| cPR | 4 (13.3) | 4 (22.2) | 2 (25.0) | 6 (26.1) |
| SD | 17 (56.7) | 7 (38.9) | 3 (37.5) | 7 (30.4) |
| PD | 7 (23.3) | 4 (22.2) | 2 (25.0) | 8 (34.8) |
| NE^{e} | 0 | 0 | 0 | 1 (4.3) |
| NA | 2 (6.7) | 3 (16.7) | 1 (12.5) | 0 |

^{a}Responses were observed in HNSCC, NSCLC, EC, and cSCC
^{b}Two-sided 95% exact confidence interval, computed using the Clopper-Pearson method (1934)
^{c}Best overall response according to RECIST v1.1 CR or PR were confirmed with repeat scans >=28 days after the initial response
^{d}cCR in NSCLC in 2Q4W
^{e}Patients had post-baseline assessment and the best overall response was determined to be NE per RECIST v1.
The efficacy-evaluable set includes all treated patients who had both a baseline and at least 1 evaluable post baseline disease assessment per RECIST v1.1 (Assessed by investigator) or discontinued the treatment.

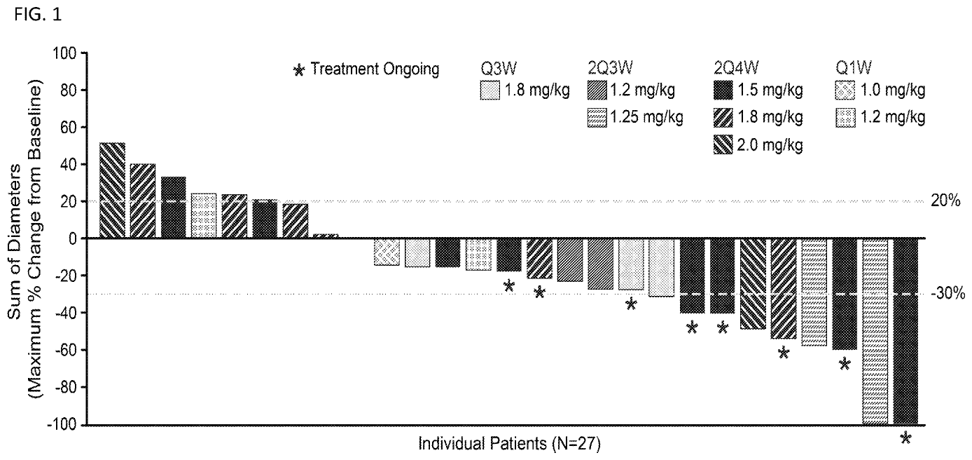

FIG. 1 is shows the antitumor activity of SGN-B6A in the NSCLC subset of the dose escalation study. Subjects had received a median of 3.5 (range: 1-8) lines of prior therapy. The objective response of the NSCLC subjects from FIG. 1 are shown in Table 7.

TABLE 7
Objective response rate of the NSCLC subset
of the dose escalation study

| | NSCLC |
|---|---|
| | Total |
| | N = 27 |
| cORR, n (%) | 9 (33.3) |
| 95% CI | (16.5, 54.0) |
| Best Overall Response | |
| (Overall, n[%]) | |
| cCR | 1 (3.7) |
| cPR | 8 (29.6) |
| SD | 8 (29.6) |
| PD | 9 (33.3) |
| NE | 1 (3.7) |
| NA | 0 |

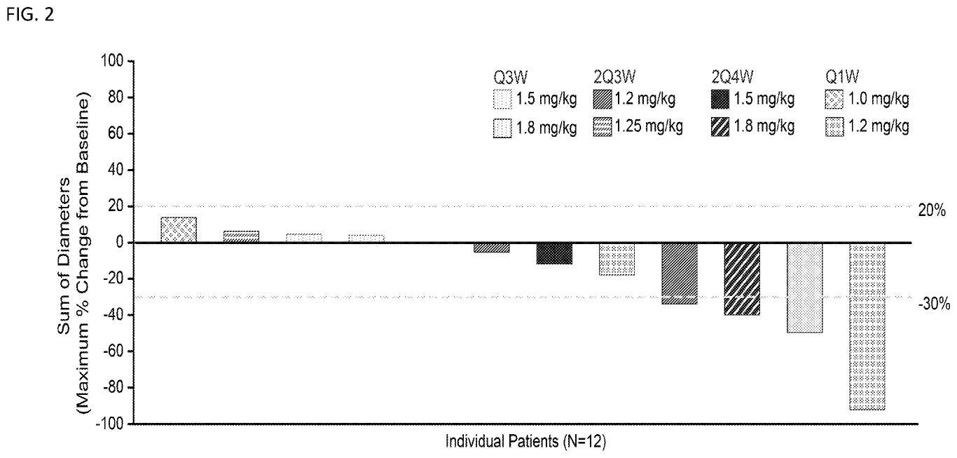

FIG. 2 shows the antitumor activity of SGN-B6A in the EC (esophageal cancer) subset of the dose escalation study. Subjects had received a median of 3.0 (range: 1-5) lines of prior therapy. The objective response of the EC subjects from FIG. 2 are shown in Table 8.

TABLE 8
Objective response rate of the EC subset
of the dose escalation study

| | EC |
|---|---|
| | Total |
| | N = 12 |
| cORR, n (%) | 4 (33.3) |
| 95% CI | (9.9, 65.1) |
| Best Overall Response | |
| (Overall, n[%]) | |
| cCR | 0 |
| cPR | 4 (33.3) |
| SD | 4 (33.3) |
| PD | 4 (33.3) |
| NE | 0 |
| NA | 0 |

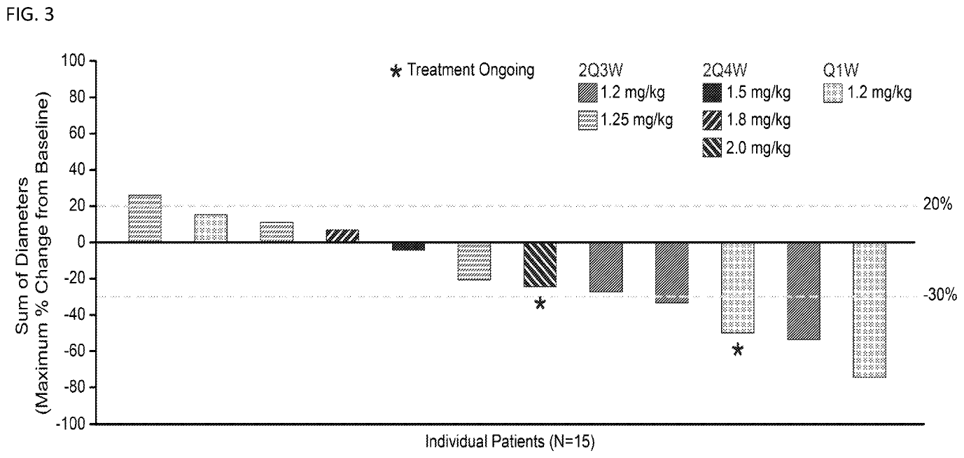

FIG. 3 shows the antitumor activity of SGN-B6A in the HNSCC subset of the dose escalation study. Subjects had received a median of 3.0 (range: 1-4) lines of prior therapy. The objective response of the HNSCC subjects from FIG. 3 are shown in Table 9.

TABLE 9
Objective response rate of the HNSCC subset
of the dose escalation study

| | HNSCC |
|---|---|
| | Total |
| | N = 15 |
| cORR, n (%) | 3 (20.0) |
| 95% CI | (4.3, 48.1) |
| Best Overall Response | |
| (Overall, n[%]) | |
| cCR | 0 |
| cPR | 3 (20.0) |
| SD | 7 (46.7) |
| PD | 3 (20.0) |
| NE | 0 |
| NA | 2 (13.3) |

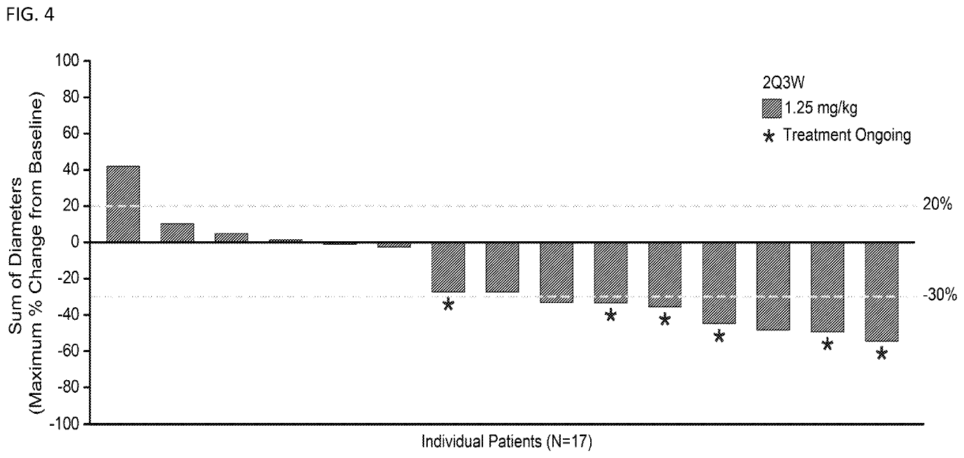

FIG. 4 shows the antitumor activity of SGN-B6A in the HNSCC 2Q3W subset of the dose expansion study. Subjects had received a median of 3.0 (range: 1-6) lines of prior therapy. The efficacy-evaluable set includes all treated patients who had both a baseline and at least 1 evaluable post baseline disease assessment per RECIST v1.1 (Assessed by investigator) or discontinued the treatment. 2 subjects are not displayed due to the lack of post baseline assessment that is eligible for the efficacy analysis. The objective response of the HNSCC subjects from FIG. 4 are shown in Table 10.

TABLE 10
Objective response rate of the HNSCC 2Q3W
subset of the dose expansion study

| | 2Q3W, |
|---|---|
| | HNSCC |
| | 1.25 mg/kg |
| | N = 17 |
| cORR, n (%) | 5 (29.4) |
| 95% CI | (10.3, 56.0) |
| Best Overall Response | |
| (Overall, n[%]) | |
| cCR | 0 |
| cPR | 5 (29.4) |
| SD | 5 (29.4) |
| PD | 5 (29.4) |
| NE | 1 (5.9) |
| NA | 1 (5.9) |

### Example 2: Analysis of SGN-B6A Treatment in Subjects with NSCLC

Subjects with NSCLC were treated as described in Example 1.

TABLE 11
ORR at various doses

| | | | 1.8 mg/kg |
|---|---|---|---|
| | 1.25 mg/kg | 1.5 mg/kq | AiBW |
| Parameter | 2Q3W | 2Q4W | 2Q4W |
| NSCLC overall | 9.7% | 23.1% | 18.5% |
| Non-squamous | 5.6% | 27.3% | 20.8% |
| NSCLC | | | |
| Taxane-naïve | 22.2% | 27.8% | 31.3% |
| NSCLC | | | |
| Non-squamous and | 16.7% | 33.3% | 31.3% |
| taxane-naïve NSCLC | | | |

TABLE 12
ORR in subjects previously treated with a taxane compared to
taxane-naïve subjects and subjects with squamous cell carcinoma
compared to subjects with non-squamous cell carcinoma

| | Squamous | Non-Squamous | |
|---|---|---|---|
| | (N = 22) | (N = 75) | Total |
| Prior taxane treatment | 1/16 (6.3%) | 4/38 (10.5%) | 5/54 (9%) |
| (N = 54) | | | |
| Taxane-naïve | 1/6 (16.7%) | 11/37 (29.7%) | 12/43 (28%) |
| (N = 43) | | | |
| Total | 2/22 (9%) | 15/75 (20%) | |

These results demonstrate that there is about a 2-fold greater response in subjects with non-squamous cell carcinoma compared to squamous cell carcinoma and about a 3-fold greater response in taxane-naïve subjects compared to subjects previously treated with a taxane.

TABLE 13
ORR in taxane-naïve subjects with non-squamous
NSCLC at various doses

| Taxane-naïve | | | | |
|---|---|---|---|---|
| and non- | 1.25 mg/kg | 1.5 mg/kg | 1.8 mg/kg | |
| squamous cell | 2Q3W | 2Q4W | AiBW 2Q4W | |
| NSCLC | (N = 6) | (N = 15) | (N = 16) | Total |
| ORR | 1/6 (16.7%) | 5/15 (33.3%) | 5/16 (31.3%) | 11/37 (29.7%) |
| 95% confidence | (0.4, 64.1) | (11.8, 61.6) | (11.0, 58.7%) | (15.9, 47.0) |
| interval | | | | |

### Example 3: Mouse Surrogate of SGN-B6A (mSGN-B6A) in Combination with Anti-mPD1 Shows Anti-Tumor Activity in a Renca Syngeneic Model Engineered to Express Human Integrin Beta-6

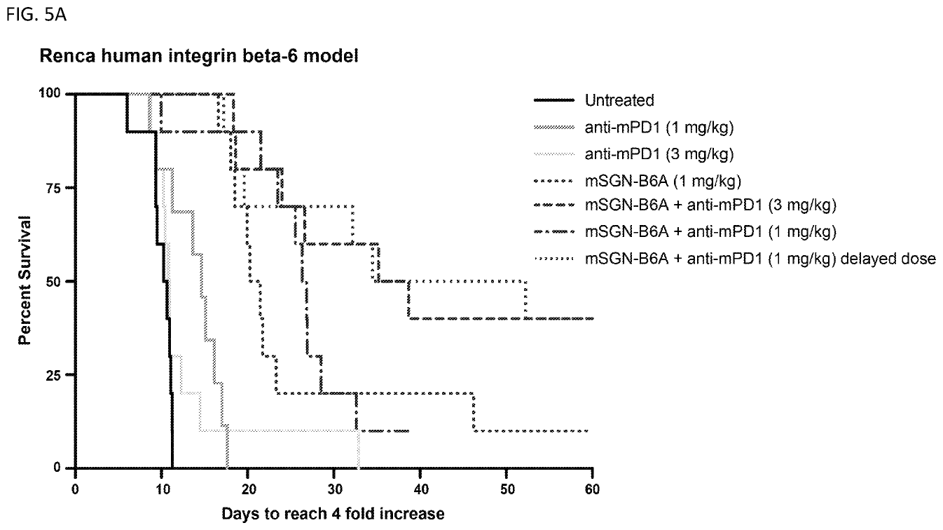

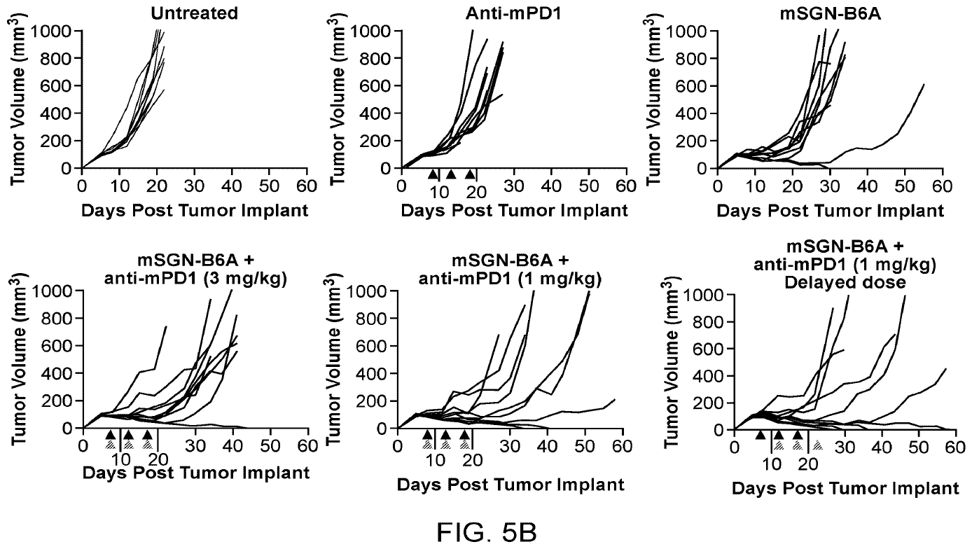

BALB/c mice (n=10 mice/group) were subcutaneously implanted with syngeneic Renca cells engineered to express human integrin beta-6. mSGN-B6A and anti-mouse PD-1 (anti-mPD1) were dosed weekly for three doses, individually or in combination. A delay dose treatment group received the first does of anti-mPD1 1 week after the first mSGN-B6A dose. FIG. 5A shows a Kaplan-Meier plot, where tumor increase is the survival endpoint. FIG. 5B and Table 11 show that individual mice treated with mSGN-B6A show 2 complete regressions observed at day 19 and 1 maintained durable complete regression until end of study (Day 58). Individual mice treated with mSGN-B6A and anti-mPD1 at 1 mg/kg had 3 CTRs at Day 19 with 1 maintained durable complete tumor regression (dCTR). Individual mice treated with mSGN-B6A and mPD1 at 3 mg/kg had 3 CTRs at Day 19 with 3 maintained dCTR. Individual animals in the delayed dose arm showed 5 CTRs at Day 19 with 4 dCTRs at end of study.

TABLE 14
Tumor growth inhibition in Renca human integrin
beta-6 syngeneic model

| | | Tumor | | | |
|---|---|---|---|---|---|
| | | Volume | | | |
| | | (mm³)^{a} | TGI (%)^{b} | CTR^{c} | dCTR^{d} |
| Treatment | Dose | (Day 19) | (Day 19) | (Day 19) | (Day 58) |
| Untreated | — | 657.4 ± 43.1 | — | 0/10 | 0/10 |
| mSGN-86A | 1 mg/kg | 139.9 ± 21.8 | 92.4 | 2/10 | 1/10 |
| Anti-mPD1 | 1 mg/kg | 469.2 ± 95.1 | 33.7 | 0/10 | 0/10 |
| mSGN-86A | 1 mg/kg | 135.3 ± 38.0 | 93.5 | 3/10 | 1/10 |
| Anti-mPD1 | 1 mg/kg | | | | |
| mSGN-86A | 1 mg/kg | 112.7 ± 26.3 | 97.0 | 3/10 | 3/10 |
| Anti-mPD1 | 3 mg/kg | | | | |
| mSGN-86A | 1 mg/kg | 98.8 ± 23.7 | 99.8 | 5/10 | 4/10 |
| Anti-mPD1 | 1 mg/kg | | | | |
| delayed dose | | | | | |

^{a}Mean ± SEM
^{b}TGI: tumor growth inhibition; TGI (%) = 100 × [1 − (Vₜᵣₑₐₜ₋ₜ − Vₜᵣₑₐₜ₋₁)/(V_{control-t} − V_{control-1})] where Vₜᵣₑₐₜ₋₁ and V_{control-1} are mean tumor volumes of the treated and control groups on grouping day, and Vₜᵣₑₐₜ₋ₜ and V_{control-t} are mean tumor volumes of the treated and control groups on a given day
^{c}CTR: complete tumor regression; number of animals with no measurable tumors (≤63 mm³)
^{d}dCTR: durable complete tumor regression; number of animals with no measurable tumors (≤63 mm³) at the end of study (Day 58)

What is claimed is:

1. A method of treating a solid tumor in a subject, the method comprising administering to the subject an antibody-drug conjugate that binds to alpha-v beta-6 (αvβ6) or an antigen-binding fragment thereof conjugated to a monomethyl auristatin or a functional analog thereof or a functional derivative thereof, wherein the antibody-drug conjugate is administered at a dose ranging from about 0.7 mg/kg to about 2.5 mg/kg of the subject's body weight, and wherein the anti-αvβ6 antibody or antigen-binding fragment of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6.

2. The method of claim 1, wherein the anti-αvβ6 antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.

3. The method of claim 1 or claim 2, wherein the anti-αvβ6 antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

4. The method of any one of claims 1-3, wherein the anti-αvβ6 antibody or antigen-binding fragment thereof of the antibody-drug conjugate comprises a heavy chain comprising the amino acid sequence of SEQ ID NO:9 and a light chain comprising the amino acid sequence of SEQ ID NO: 10.

5. The method of any one of claims 1-4, wherein the monomethyl auristatin is monomethyl auristatin E (MMAE).

6. The method of any one of claims 1-5, wherein the anti-αvβ6 antibody or antigen-binding fragment thereof of the antibody-drug conjugate is a monoclonal antibody or a monoclonal antigen-binding fragment thereof.

7. The method of any one of claims 1-6, wherein the antibody-drug conjugate further comprises a linker between the anti-αvβ6 antibody or antigen-binding fragment thereof and the monomethyl auristatin.

8. The method of claim 7, wherein the linker is a cleavable peptide linker.

9. The method of claim 8, wherein the cleavable peptide linker has a formula: -MC-vc-PAB-, wherein:
a) MC is: 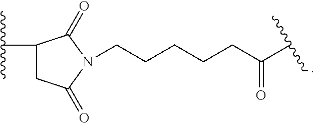 [SMILES: *C(=O)CCCCCN1C(=O)CC(*)C1=O]
b) vc is the dipeptide valine-citrulline, and
c) PAB is:

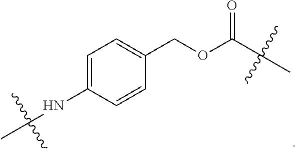 [SMILES: *Nc1ccc(COC(*)=O)cc1]

10. The method of any one of claims 7-9, wherein the linker is attached to sulphydryl residues of the anti-αvβ6 antibody obtained by partial reduction or full reduction of the anti-αvβ6 antibody or antigen-binding fragment thereof.

11. The method of claim 10, wherein the linker is attached to monomethyl auristatin E (MMAE), wherein the antibody-drug conjugate has the following structure: 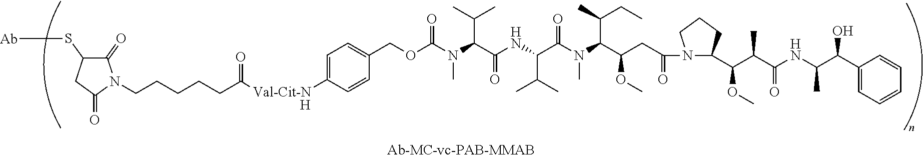
wherein p denotes a number from 1 to 8, S represents a sulphydryl residue of the anti-αvβ6 antibody, and Ab designates the anti-αvβ6 antibody or antigen-binding fragment thereof.

12. The method of claim 11, wherein the average value of p in a population of the antibody-drug conjugates is about 4.

13. The method of any one of claims 1-12, wherein the dose is about 0.8 mg/kg, about 1.0 mg/kg, about 1.2 mg/kg, about 1.25 mg/kg, about 1.5 mg/kg, about 1.8 mg/kg, about 2.0 or about 2.4 mg/kg of the subject's body weight.

14. The method of any one of claims 1-12, wherein the dose is 0.8 mg/kg, 1.0 mg/kg, 1.2 mg/kg, 1.25 mg/kg, 1.5 mg/kg, 1.8 mg/kg, 2.0 or 2.4 mg/kg of the subject's body weight.

15. The method of claim 14, wherein the dose is 1.8 mg/kg of the subject's body weight.

16. The method of claim 14, wherein the dose is 1.25 mg/kg of the subject's body weight.

17. The method of any one of claims 1-16, wherein the subject's body weight is the subject's ideal body weight (IBW).

18. The method of any one of claims 1-16, wherein the subject's body weight is the subject's adjusted ideal body weight (AIBW).

19. The method of any one of claims 1-18, wherein the antibody-drug conjugate is administered once about every 1 week, once about every 2 weeks, once about every 3 weeks, or twice about every 3 weeks.

20. The method of claim 19, wherein the antibody-drug conjugate is administered once about every 2 weeks.

21. The method of claim 19, wherein the antibody-drug conjugate is administered is once about every 1 week for 2 consecutive weeks followed by about a 1 week resting period without any administration of the antibody-drug conjugate so that each cycle time is about 21 days including the resting period.

22. The method of claim 21, wherein the antibody drug conjugate is administered on Days 1 and 8 of each 21-day cycle with no administration on Day 15.

23. The method of any one of claims 1-22, wherein the subject has been previously treated for the solid tumor with one or more therapeutic agents and did not respond to treatment, relapsed after the treatment, or experienced disease progression during the treatment, wherein the one or more therapeutic agents is not the antibody-drug conjugate.

24. The method of any one of claims 1-23, wherein the solid tumor is locally advanced or metastatic.

25. The method of any one of claims 1-24, wherein the solid tumor is selected from the group consisting of lung cancer, head and neck cancer, breast cancer, esophageal cancer, gastroesophageal junction cancer, ovarian cancer, peritoneal cancer, or fallopian tube cancer, skin cancer, pancreatic cancer, bladder cancer, cervical cancer, and gastric cancer.

26. The method of claim 25, wherein the lung cancer is non-small cell lung cancer.

27. The method of claim 26, wherein the non-small cell lung cancer a non-squamous cell carcinoma.

28. The method of claim 26, wherein the non-small cell lung cancer is a squamous cell carcinoma.

29. The method of any one of claims 25-28, wherein the solid tumor is lung cancer and the lung cancer does not have a known epidermal growth factor receptor (EGFR) or anaplastic lymphoma kinase (ALK) mutation.

30. The method of any one of claims 25-28, wherein the solid tumor is lung cancer and the lung cancer has a known EGFR or ALK mutation.

31. The method of any one of claims 25-30, wherein the solid tumor is lung cancer and the subjected received prior platinum-based therapy and prior therapy with a PD-1/PD-L1 inhibitor.

32. The method of any one of claims 25-31, wherein the solid tumor is lung cancer and the subject has not received prior therapy with a taxane.

33. The method of claim 25, wherein the head and neck cancer is head and neck squamous cell cancer.

34. The method of claim 25 or claim 33, wherein the solid tumor is head and neck cancer and the subjected received prior platinum-based therapy and prior therapy with a PD-1/PD-L1 inhibitor.

35. The method of claim 25, wherein the breast cancer is HER2-negative breast cancer.

36. The method of claim 25 or 35, wherein the solid tumor is breast cancer and the subject received 1 or more prior lines of therapy for the breast cancer.

37. The method of claim 36, wherein the 1 or more prior lines of therapy included a taxane administered as either a single agent or in combination with a different agent.

38. The method of claim 25, wherein the esophageal cancer is esophageal squamous cell carcinoma.

39. The method of claim 25 or 38, wherein the solid tumor is esophageal cancer and the subject received prior platinum-based chemotherapy.

40. The method of claim 25, wherein the esophageal cancer is esophageal adenocarcinoma.

41. The method of claim 25, wherein the gastroesophageal junction cancer is gastroesophageal junction adenocarcinoma.

42. The method of claim 40 or claim 41, wherein the subject received prior platinum-based chemotherapy.

43. The method of claim 25, wherein the ovarian cancer is high grade serous epithelial ovarian cancer.

44. The method of claim 25, wherein the skin cancer is cutaneous squamous cell carcinoma.

45. The method of claim 25 or claim 44, wherein the solid tumor is skin cancer and the subject received prior therapy with a PD-1/PD-L1 inhibitor.

46. The method of claim 25, wherein the pancreatic cancer is exocrine pancreatic adenocarcinoma.

47. The method of any one of claims 1-46, wherein the solid tumor is an advanced stage cancer.

48. The method of claim 47, wherein the advanced stage cancer is a stage 3 or stage 4 cancer.

49. The method of claim 47 or 48, wherein the advanced stage cancer is metastatic cancer.

50. The method of any one of claims 1-49, wherein the route of administration for the antibody-drug conjugate is intravenous.

51. The method of any one of claims 1-50, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express αvβ6.

52. The method of any one of claims 1-51, wherein one or more therapeutic effects in the subject is improved after administration of the antibody-drug conjugate relative to a baseline.

53. The method of claim 52, wherein the one or more therapeutic effects is selected from the group consisting of: size of a tumor derived from the cancer, objective response rate, duration of response, time to response, progression free survival, and overall survival.

54. The method of any one of claims 1-53, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate.

55. The method of any one of claims 1-54, wherein the objective response rate is at least about 20%, at least about 25%, at least about 30%, at least about 35%, at least about 40%, at least about 45%, at least about 50%, at least about 60%, at least about 70%, or at least about 80%.

56. The method of any one of claims 1-55, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

57. The method of any one of claims 1-56, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

58. The method of any one of claims 1-57, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

59. The method of any one of claims 1-58, wherein the subject has one or more adverse events and is further administered an additional therapeutic agent to eliminate or reduce the severity of the one or more adverse events.

60. The method of any one of claims 1-59, wherein the subject is at risk of developing one or more adverse events and is further administered an additional therapeutic agent to prevent or reduce the severity of the one or more adverse events.

61. The method of any one of claims 1-60, wherein the antibody-drug conjugate is administered as a monotherapy.

62. The method of any one of claims 1-60, wherein the method further comprises administering to the subject one or more additional therapeutic agents.

63. The method of claim 62, wherein the one or more additional therapeutic agents is a checkpoint inhibitor.

64. The method of claim 63, wherein the checkpoint inhibitor is a PD-1 inhibitor or a PD-L1 inhibitor.

65. The method of claim 64, wherein the PD-1 inhibitor is an anti-PD-1 antibody.

66. The method of claim 65, wherein the anti-PD-1 antibody is pembrolizumab, or a biosimilar thereof.

67. The method of claim 66, wherein the pembrolizumab or biosimilar thereof is administered at a dose of about 200 mg.

68. The method of claim 66 or claim 67, wherein the pembrolizumab or biosimilar thereof is administered once about every 3 weeks.

69. The method of claim 66, wherein the pembrolizumab or biosimilar thereof is administered at a dose of about 400 mg.

70. The method of claim 66 or claim 69, wherein the pembrolizumab or biosimilar thereof is administered once about every 6 weeks.

71. The method of any one of claims 66-70, wherein the route of administration of the pembrolizumab or biosimilar thereof is intravenous.

72. The method of any one of claims 63-71, wherein prior to the treatment the tumor comprises one or more cells that express PD-L1.

73. The method of claim 72, wherein, the subject has a tumor that expresses PD-L1 with TPS≥1%.

74. The method of claim 72, wherein, the subject has a tumor that expresses PD-L1 with TPS≥20%.

75. The method of claim 72, wherein the subject has a tumor that expresses PD-L1 with TPS≥50%.

76. The method of claim 72, wherein the subject has a tumor that expresses PD-L1 with CPS≥1.

77. The method of claim 72, wherein the subject has a tumor that expresses PD-L1 with CPS≥20.

78. The method of any one of claims 62-77, wherein the one or more additional therapeutic agents is a platinum-based agent.

79. The method of claim 78, wherein the platinum-based agent is carboplatin or cisplatin.

80. The method of any one of claims 62-79, wherein the first dose of the antibody-drug conjugate is administered prior to the administration of the first dose of the one or more additional therapeutic agents.

81. The method of claim 80, wherein the first dose of the antibody drug conjugate is administered at least 1 day, at least 2 days, at least 3 days, at least 4 days, at least 5 days, at least 6 days, at least 7 days, at least 2 weeks, at least 3 weeks, at least 4 weeks, at least 2 months, at least 3 months, at least 4 months, at least 5 months, or at least 6 months prior to the administration of the first dose of the one or more additional therapeutic agents.

82. The method of any one of claims 1-82, wherein the subject is a human.

83. The method of any one of claims 1-83, wherein the antibody-drug conjugate is in a pharmaceutical composition comprising the antibody-drug conjugate and a pharmaceutical acceptable carrier.

84. A kit comprising:
(a) a dosage ranging from about 0.7 mg/kg to about 2.5 mg/kg of an antibody-drug conjugate that binds to αvβ6, wherein the antibody-drug conjugate comprises an anti-αvβ6 antibody or an antigen-binding fragment thereof conjugated to a monomethyl auristatin or a functional analog thereof or a functional derivative thereof; and
(b) instructions for using the antibody drug conjugate according to the method of any one of claims 1-83.

85. Use of an antibody-drug conjugate that binds to αvβ6 for the manufacture of a medicament for use in the method of any one of claims 1-83, wherein the antibody-drug conjugate comprises an anti-αvβ6 antibody or an antigen-binding fragment thereof conjugated to a monomethyl auristatin or a functional analog thereof or a functional derivative thereof.

86. An antibody-drug conjugate that binds to αvβ6 for use in the method of any one of claims 1-83, wherein the antibody-drug conjugate comprises an anti-αvβ6 antibody or an antigen-binding fragment thereof conjugated to a monomethyl auristatin or a functional analog thereof or a functional derivative thereof.
